(19) United States
(12) Patent Application Publication
(10) Pub. No.: US 2010/0267576 A1
(43) Pub. Date: Oct. 21, 2010

(54) Compositions And Methods For Identifying And Treating Subjects At Risk Of Developing Type 2 Diabetes

(75) Inventors: Kang Zhang, Salt Lake City, UT (US); Zhenglin Yang, Salt Lake City, UT (US); Jantje M. Gerdes, Baltimore, MD (US); Nicholas Katsanis, Perry Hall, MD (US)

Correspondence Address: Ballard Spahr LLP, SUITE 1000, 999 PEACHTREE STREET, ATLANTA, GA, 30309-3915, US

(73) Assignees: University of Utah Research foundation, Salt LAKE CITY, UT (US); The John Hopkins University, Baltimore, MD (US)

(21) Appl. No.: 12/596,892

(22) PCT Filed: May 2, 2008
(86) PCT No.: PCT/US08/62353

§ 371 Date: Jun. 30, 2010

Related U.S. Application Data
(60) Provisional application No. 60/915,585, filed on May 2, 2007.

(51) Int. Cl. C12Q 1/68; C40B 30/04

(52) U.S. Cl. 506/9; 435/6

(57) ABSTRACT

Disclosed herein are compositions and methods for the identification of a subject at risk for developing type 2 diabetes. Also disclosed is a therapeutic target for the prevention and treatment of type 2 diabetes.

### CROSS-REFERENCE TO RELATED APPLICATIONS

This application claims benefit of U.S. Provisional Application No. 60/915,585, filed May 2, 2007, which is hereby incorporated herein by reference in its entirety.

### STATEMENT REGARDING FEDERALLY SPONSORED RESEARCH

This invention was made with government support under Grants R01EY14448, R01EY14428, P30EY014800, GCRC M01-RR00064, HD04260, DK072301, DK075972, and DK54931 awarded by the National Institutes of Health. The government has certain rights in the invention.

### BACKGROUND

Type II Diabetes Mellitus (T2DM) represents a major global health challenge, with over 170 million affected individuals in the general population, a figure that is expected to double over the next 20 years (Permutt, M. A., et al. 2005). Genetic and population-based studies have shown T2DM to be a complex trait, likely resulting from the interaction of genetic susceptibility alleles and environmental influence. Although genetic factors appear to be important in the susceptibility (or resistance) to T2DM, the causal genes remain largely unknown. To identify such loci as a means to both assist in the management of T2DM in the population and as a way to determine further the underlying etiopathology of the disease, a variety of approaches have been implemented, ranging from dissection of monogenic forms of diabetes to, more recently, genome-wide approaches in which new genomic tools (such as HapMap; Altshuler, D., et al. 2005) and high-throughput genotyping technologies have been combined to interrogate large T2DM patient cohorts. As a result of such efforts, several potential T2DM susceptibility loci have been identified, including potentially causal variants in CAPN10 (Horikawa, Y., et al. 2000), ENPP1 (Meyre, D., et al. 2005) and HNF4A (Weedon, M. N., et al. 2004; Silander, K., et al. 2004). Another independent study also suggested FTO as a T2DM susceptibility locus; however this signal is more likely associated with obesity (Frayling T M., et al. 2007), a major susceptibility factor to T2DM development, underpinning the relationship between the pathogenesis of T2DM and other metabolic defects. Most recently, a large-scale whole genome association study implicated a novel locus, SLC30A8, as well as two haplotype blocks that likely contain additional susceptibility genes (Sladek, R., et al. 2007). Candidate gene association studies have also highlighted several potential loci, but only PPARG and KCNJ11 have been replicated consistently. To date, the most convincingly replicated gene for T2DM is TCF7L2, a predicted transcription factor with target sites for β-catenin (Sladek, R., et al. 2007; Grant, S., et al. 2006; Helgason, A., et al. 2007). Thus, needed are genetic markers for identifying subjects at risk of developing type 2 diabetes, as well as methods of treating and preventing type II diabetes.

### BRIEF SUMMARY

In accordance with the purpose of this invention, as embodied and broadly described herein, this invention relates to compositions and methods for identifying a subject at risk for developing type 2 diabetes and methods of treating or preventing same.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows association of SNPs in DKK3 with T2DM. FIG. 1A shows log p-values (Y-axis) from association analyses for 14 SNPs in the chromosome 11 region harboring DKK3 gene. Diamonds represent −log p values of all Utah cohort SNPs. Black circles represent −log p values of all SNPs genotyped in the southern Chinese cohort. The square indicates the −log p value for rs11022111 in the Southern Chinese cohort. All rs11022111 SNPs are labeled. Black circle at rs11022111 indicates −log p value of the combined Chinese southern and northern cohorts. FIG. 1B shows genomic structure and locations of genes between 11928 kb and 12028 kb (UCSC build 35). FIG. 1C shows pairwise D′ Hapview plot for SNPs in chromosome 11 region round DKK3 gene using samples from Utah case cohort.

FIG. 2 shows expression of DKK3 in humans and mice. FIG. 2A shows effects of the rs11022111 variants on luciferase reporter expression in cultured HEK293 cells. pGL3 luciferase reporter recombinant plasmids containing a DKK3 promoter sequence with the risk allele C (rs11022111-C construct) or normal G allele (rs11022111-G construct) at SNP rs11022111, the pGL3-Basic vector without insert (negative control) was transfected in HEK 293 cells. Renilla luciferase plasmid pTK-RL was cotransfected with each construct as an internal control. Normalized luciferase activity was measured in twelve independent experiments. Expression value of rs11022111-G allele was set at 1. The mean±SD in given for each construct. Significance was examined using SPSS's independent samples t-test. The error bars indicate the 95.0% confidence interval of the mean. FIG. 2B shows Real Time RT-PCR quantitative analysis of DKK3 RNA levels derived from kidneys of 7 db/db mice and 7 normal littermate controls. Each RT-PCR was run in duplicates. Significance was examined using SPSS's independent samples t-test. The error bars indicate the 95.0% confidence interval of the mean.

FIG. 3 shows DKK3 is a likely Wnt agonist. Wnt3a stimulated, β-catenin dependent luciferase activity in 293T cells upon suppression (sh) of DKK3, and in presence of endogenous (ev) and excess (o/e) levels of DKK3 reveals the influence of LRP6 and either Fz5, Fz7, or Fz8 on DKK3 mediated Wnt signaling. The stimulated (+Wnt3a) levels were normalized against the unstimulated (−Wnt3a) levels to correct for the Wnt3a dependent portion of transmitted signal. Arrows point to significantly downregulated β-catenin activity upon loss of DKK3 compared to endogenous levels.

FIG. 4 shows in vivo Wnt phenotypes upon suppression or overexpression of dkk3. FIG. 4A shows live embryos shown ventrally (upper panels) and dorsally (lower panels) were injected with 5 ng (Class I) and 1 ng (ClassII) dkk3 morpholino. Embryos were staged around ten somites. Note the double axis formation (arrows) in overexpressants with high doses of dkk3 mRNA. Arrowheads are shown to emphasize the widened body gap angle which is corresponding to shortened body axis of the embryos. Asterisks and bars point out the widened notochord and dorso-laterally elongated somites, indicative of defective C+E movements. FIG. 4B shows expression domains of both chordin and goosecoid are expanded in embryos overexpressing dkk3 compared to wt animals, indicative of dorsalization.

FIG. 5 shows DKK3 is upstream of TCF7L2. FIG. 5A shows suppression of DKK3 results in loss of TCF7L2 message in presence of LRP6 and Fz5, Fz7, or Fz8 while excess DKK3 results in increased TCF7L2 transcription in presence of LRP6 and Fz5 or Fz8 but not Fz7, no effect on TCF7L2 transcription was observed for Fz1, Fz2, or Fz4. FIG. 5B shows vo-injection of tcf712 (3 ng) and dkk-3 morpholino (1 ng) produce ˜70% affected embryos (compared to ˜10% and 15% respectively for the individual morphants), which implicates an additive effect of the two genes. FIG. 5C shows plot of odds ratios (ORs) for individuals carrying a risk TCF7L2 allele, a DKK3 risk allele, or alleles at both loci, also showing a potentially additive effect.

FIG. 6 shows evaluation of the efficacy of shRNAs against human DKK3 in embryonic fibroblasts. Real-time RT-PCR of DKK3 mRNA show the relative levels of steady state message 72 h post transfection and demonstrate the relative potency of the three shRNA plasmids.

FIG. 7 shows effect of DKK3 expression on Wnt activity mediated by a combination of Fz1-10 with either LRP5 or LRP6. FIG. 7A shows TOPFlash luciferase activity in 293T cells transiently expressing shDkk3, Dkk3, and Fz1-8. FIG. 7B shows Wnt3a specific, β-catenin dependent luciferase activity in 293T cells upon suppression (sh) of DKK3, and in presence of endogenous (ev) and excess (o/e) levels of DKK3 reveals the influence of LRP5 and either Fz1, Fz2, Fz4, Fz5, Fz7, or Fz8 on DKK3 mediated Wnt signaling. FIG. 7C shows same as 7B with stimulated (+Wnt3a) levels normalized against the unstimulated (−Wnt3a) levels to correct for the Wnt3a dependent portion of transmitted signal.

FIG. 8 shows effect of DKK3 expression on Wnt activity mediated by a combination of Fz1-10 with LRP6. FIG. 8A shows Wnt3a specific, β-catenin dependent luciferase activity in 293T cells upon suppression (sh) of DKK3, and in presence of endogenous (ev) and excess (o/e) levels of DKK3 reveals the influence of LRP6 and either Fz1, Fz2, Fz4, Fz5, Fz7, or Fz8 on DKK3 mediated Wnt signaling. The stimulated (+Wnt3a) levels were normalized against the unstimulated (−Wnt3a) levels to correct for the Wnt3a dependent portion of transmitted signal. FIG. 8B shows excessive DKK3 does not significantly alter β-catenin dependent transcriptional activity mediated by Fz and LRP6.

FIG. 9 shows titration and rescue of dkk3 morphants. FIG. 9A shows severity and frequency of the dkk3 morpholino phenotype is dosage dependent. Mildly to moderately affected embryos were classified as Class I when they displayed a shortened body axis, mediolaterally elongated somites, and notochord imperfections. Class II embryos were more severely affected as defined by severely shortened body axes, bubbling of cells, mediolaterally elongated somites, and widened and kinked notochord. FIG. 9B shows rescue titration of the dkk-3 phenotype. Dkk-3 morpholino (3 ng) was co-injected with 10-50 ng of dkk-3 RNA as well as by itself. Co-injection produced only ˜15% affected embryos (compared to >35%). FIG. 9C shows expression of axin2, a β-catenin target, in zebrafish embryos. Suppression of dkk3 (dkk3MO) leads to the downregulation of axin2, whereas overexpression (dkk3o/e) has the converse effect. Standard error bars are shown (experiment was performed in triplicate). FIG. 9D shows flat mounted RNA in situ hybridization with krox20/pax2/myoD of dkk3 morphants and overexpressants. Bars demonstrate the shortened distance between 3^{rd} rhombomere and 1^{st} somite, indicative of a PCP defect. FIG. 9E shows suppression of dkk3 does not expand dorsal structures. RNA in situ hybridization with antisense probes against the dorsal markers chordin and goosecoid shows no expansion of the expression domain of either message in dkk3 morphants at 50% epiboly.

FIG. 10 shows suppression (sh) or overexpression (o/e) of DKK3 does not influence TCF7L2 message in presence of LRP5 and Fz1, Fz2, Fz3, Fz7 or Fz8.

FIG. 11 shows DKK3 immunohistochemistry in mouse pancreas and adipose tissues. FIGS. 10A-C show immunolabeling of Alpha-cells (arrows). FIG. 10A shows staining with a polyclonal antibody to glucagan (10A); FIG. 10C shows immunostaining of DKK3 in both alpha and beta cells in the pancreas; FIG. 10B shows merged pictures of 5A and 5C. FIG. 10D shows negative control omitting the primary antibody. Scale bars=20 μm. FIG. 10E shows adipocytes are immunolabeled with DKK3 antibody. The arrow heads point to adipocytes (with characteristic dark vacuum in the center represents dissolved lipid). FIG. 10F shows negative control omitting the primary antibody.

FIG. 12 shows DKK3 335R is a functional null allele. β-catenin/TCF-dependent transcriptional activity in presence of LRP5 and Fzd8 increases upon overexpression of the DKK3 335G but not 335R allele as shown by Luciferase TOPFlash assay (A). Co-injection of 3 ng dkk-3 morpholino with 50 ng of either DKK3 335G or 335R RNA results in rescue of the observed C&E phenotypes for the 335G (B, C, D) but not for the 335R (B, E, F) allele. Embryos were scored in a double-blind experiment based on parameters including widening or kinking/undulation of the notochord, body axis length and distance between the 5^{th} rhombomere and 1^{st} somite. Dorsal and lateral views of representative examples of dkk-3 MO/DKK3 335G (C, D) and dkk-3/DKK3 335R (E,F) injected embryos.

FIG. 13 shows HapMap SNPs in the DKK3 interval fail to detect association between DKK3 and T2DM. The LD block was generated using 36 SNPs from Affymetrix 500k and/or Illumina 317k chips in DKK3 region and HapMap CEU genotype data. Genotyping of our Utah cohort for 12 HapMap SNPs show no association. The relative position of the SNPs and their p-values are shown, as is the haplotype structure of the region, which fails to capture the risk DKK3 haplotype.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a oligonucleotide is disclosed and discussed and a number of modifications that can be made to a number of molecules including the oligonucleotide are discussed, each and every combination and permutation of oligonucleotide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. Diabetes

Current prevalence of type 2 diabetes (T2DM) in the world is expected to increase dramatically by the year 2030. T2DM is a complex disease resulting from interaction of genetic predisposition and environmental influence. However, the precise gene(s) involved remain largely unknown. As disclosed herein, the C allele of the single nucleotide polymorphism (SNP) rs11022111 in the promoter region of the dkk3 gene is significantly associated with T2DM in Chinese and Caucasian cohorts. As disclosed herein, DKK3 levels are significantly decreased with inheritance of the C risk allele in T2DM patients. Also as disclosed herein, DKK3 is critical in transducing Wnt signaling mediated by FZ5, FZ7, FZ8 and LRP6. Thus, disclosed herein are compositions and methods for identifying subjects at risk for developing type 2 diabetes mellitus.

The World Health Organization recognizes three main forms of diabetes: type 1, type 2, and gestational diabetes (occurring during pregnancy), which have similar signs, symptoms, and consequences, but different causes and population distributions. Type 1 is usually due to autoimmune destruction of the pancreatic beta cells which produce insulin. Type 2 is characterized by tissue-wide insulin resistance and varies widely; it sometimes progresses to loss of beta cell function. Gestational diabetes is similar to type 2 diabetes, in that it involves insulin resistance. The hormones of pregnancy cause insulin resistance in those women genetically predisposed to developing this condition. Types 1 and 2 are incurable chronic conditions, but have been treatable since insulin became medically available in 1921. Gestational diabetes typically resolves with delivery. Thus, in some aspects of the disclosed method, the subject has been diagnosed with type 1 or type 2 diabetes mellitus or gestational diabetes.

Diabetes can cause many complications. Acute glucose level abnormalities may occur if insulin level is not well-controlled. Serious long-term complications include cardiovascular disease (doubled risk), chronic renal failure (the main cause of dialysis in developed world adults), retinal damage (which can lead to blindness and is the most significant cause of adult blindness in the non-elderly in the developed world), nerve damage (of several kinds), and microvascular damage, which may cause erectile dysfunction (impotence) and poor healing. Poor healing of wounds, particularly of the feet, can lead to gangrene which can require amputation—the leading cause of non-traumatic amputation in adults in the developed world.

Diabetes, without qualification, usually refers to diabetes mellitus, but there are several rarer conditions also named diabetes. The most common of these is diabetes insipidus (unquenchable diabetes) in which the urine is not sweet; it can be caused by either kidney (nephrogenic DI) or pituitary gland (central DI) damage.

There are several rare causes of diabetes mellitus that do not fit into type 1, type 2, or gestational diabetes, namely genetic defects in beta cells (autosomal or mitochondrial), genetically-related insulin resistance, with or without lipodystrophy (abnormal body fat deposition), diseases of the pancreas (e.g. chronic pancreatitis, cystic fibrosis), hormonal defects, and chemicals or drugs. In addition, the tenth version of the International Statistical Classification of Diseases (ICD-10) contained a diagnostic entity named “malnutrition-related diabetes mellitus” (MRDM or MMDM, ICD-10 code E12).

The classical triad of diabetes symptoms is polyuria (frequent urination), polydipsia (increased thirst and consequent increased fluid intake) and polyphagia (increased appetite). These symptoms may develop quite fast in type 1, particularly in children (weeks or months) but may be subtle or completely absent—as well as developing much more slowly—in type 2. In type 1 there may also be weight loss (despite normal or increased eating) and irreducible fatigue. These symptoms may also manifest in type 2 diabetes in patients whose diabetes is poorly controlled.

Diabetes mellitus is characterized by recurrent or persistent hyperglycemia, and is diagnosed by demonstrating any one of the following:

fasting plasma glucose level at or above 126 mg/dL (7.0 mmol/l);

plasma glucose at or above 200 mg/dL or 11.1 mmol/l two hours after a 75 g oral glucose load as in a glucose tolerance test;

random plasma glucose at or above 200 mg/dL or 11.1 mmol/l.

Patients with fasting sugars between 6.1 and 7.0 mmol/l (ie, 110 and 125 mg/dL) are considered to have “impaired fasting glucose” and patients with plasma glucose at or above 140 mg/dL or 7.8 mmol/l two hours after a 75 g oral glucose load are considered to have “impaired glucose tolerance.” “Prediabetes” is either impaired fasting glucose or impaired glucose tolerance; the latter in particular is a major risk factor for progression to full-blown diabetes mellitus as well as cardiovascular disease. Thus, in some aspects, the subject has been diagnosed with pre-diabetes.

While not generally used for diagnosis, an elevated level of glucose irreversibly bound to hemoglobin (termed glycosylated hemoglobin, Hb_{A1c}, or A1C) of 6.0% or higher (the 2003 revised U.S. standard) is considered abnormal. HbA1c is primarily used as a treatment-tracking test reflecting average blood glucose levels over the preceding 90 days (approximately). However, some physicians may order this test at the time of diagnosis to track changes over time. The current recommended goal for HbA1c in patients with diabetes is <7.0%, which as defined as “good glycemic control”, although some guidelines are stricter (<6.5%). People with diabetes who have HbA1c levels within this range have a significantly lower incidence of complications from diabetes, including retinopathy and diabetic nephropathy.

Thus, in some aspects, the subject has been diagnosed with diabetes or pre-diabetes. Thus, in some aspects, the subject has a fasting plasma glucose level of at least 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 mg/dL. Thus, in some aspects, the subject has a plasma glucose of at least 140, 150, 160, 170, 180, 190, 200, 210, 220, 230, 240, 250, 260, 270, 290 or 300 mg/dL two hours after a 75 g oral glucose load in a glucose tolerance test. Thus, in some aspects, the subject has a random plasma glucose of at least 140, 150, 160, 170, 180, 190, 200, 210, 220, 230, 240, 250, 260, 270, 290 or 300 mg/dL. Thus, in some aspects, the subject has a hemoglobin Hb_{A1C} (A1C) level greater than 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0 percent.

In other aspects, the subject does not have clinical indications of diabetes. Thus, in some aspects, the subject has a fasting plasma glucose level of less than 126, 125, 124, 123, 121, 120, 115, 110, 105, 100, 95, 90, 85, or 80 mg/dL. Thus, in some aspects, the subject has a plasma glucose of less than 200, 195, 190, 185, 180, 175, 170, 165, 160, 155, 150, 145, 140, 135, or 130 mg/dL two hours after a 75 g oral glucose load in a glucose tolerance test. Thus, in some aspects, the subject has a random plasma glucose of at less than 200, 195, 190, 185, 180, 175, 170, 165, 160, 155, 150, 145, 140, 135, or 130 mg/dL. Thus, in some aspects, the subject has a hemoglobin Hb_{A1C} (A1C) level less than 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, or 4.0 percent.

Chronic elevation of blood glucose level leads to damage of blood vessels. In diabetes, the resulting problems are grouped under “microvascular disease” (due to damage to small blood vessels) and “macrovascular disease” (due to damage to the arteries). The damage to small blood vessels leads to a microangiopathy, which can cause diabetic retinopathy and/or diabetic nephropathy. Angiopathy means disease of the blood vessels (arteries, veins, and capillaries). In microangiopathy, the walls of very small blood vessels (capillaries) become so thick and weak that they bleed, leak protein, and slow the flow of blood. For example, diabetics can develop microangiopathy with thickening of capillaries in many areas including the eye.

Diabetic retinopathy refers to growth of friable and poor-quality new blood vessels in the retina as well as macular edema (swelling of the macula), which can lead to severe vision loss or blindness. Retinal damage (e.g., from microangiopathy) makes it the most common cause of blindness among non-elderly adults in the US.

Diabetic nephropathy refers to damage to the kidney which can lead to chronic renal failure, eventually requiring dialysis. Diabetes mellitus is the most common cause of adult kidney failure worldwide in the developed world.

The disclosed method generally comprises detecting in a sample of nucleic acid from a subject a cystidine at nucleotide position 11987669 on chromosome 11 (according to the March 2006 assembly of the human genome), which is located 965 by upstream of the dkk3 transcription start site.

### B. Compositions

1. DKK3

The Dickkopf (Dkk) family of secreted proteins consists of four members, which share two conserved cysteine-rich domains. The hallmark of Dkk proteins is that they function as Wnt antagonists or agonists by binding to and inhibiting or activating the Wnt coreceptor LRP6. They show regionalized expression during vertebrate embryogenesis. Dkk1 is the best-characterized member of the family. It acts as an embryonic head inducer, and when overexpressed it will induce extra heads in Xenopus and zebra fish. Dkk1 mutant mice are embryonic lethal, and embryos lack anterior head structure and display fused digits. Dkk2 mouse mutants are viable but show bone defects. Little is known about the biological role of Dkk4.

By a number of criteria, dkk3 appears as a divergent member of the Dkk family. Unlike Dkk1, -2, and -4, Dkk3 is not known to act as a Wnt modulator. While all other Dkk proteins bind to and modulate the Wnt receptor LRP6, as well as the Dkk coreceptor Kremen, Dkk3 has no known affinity to these transmembrane proteins, and no other proteins are known to interact with it.

Like other Dkk members, Dkk3 is expressed during vertebrate development in suggestive patterns in many organs. Dkk3 has been proposed to act as a tumor suppressor, as it is downregulated in a number of tumor cells and since dkk3 overexpression suppresses cell growth. Hence, Dkk3 is also known as REIC (for reduced expression in immortalized cells). While hypermethylation of human Dkk3 correlates with certain cancers, the physiological relevance of altered Dkk3 expression in tumors and its potential growth inhibitory effect are unknown.

2. Wnt Signaling

The Wnt ligands are a family of 19 molecules that are secreted, vary in length between 350 and 400 amino acids (aa), possess 22 to 24 conserved cysteines, and show 20 to 85% amino acid identity within the family. In general, there are three signaling pathways associated with Wnt-receptor interaction. The first is commonly called the canonical pathway. The heart of this pathway centers on β-Catenin. β-Catenin, in an active form, is a transcriptional activator for the TCF (T cell factor)/LEF-1 (lymphoid enhancer factor 1) family of DNA binding proteins. Examples of TCF-responsive genes include c-myc and cyclin D1. The pentameric regulatory complex is composed of β-Catenin, axin (axis inhibition), CK-1, APC (adenomatous polyposis coli protein), and GSK-3β (glycogen synthase kinase 3β). In the absence of Wnt, GSK-3β constitutively phosphorylates β-Catenin, targeting it for degradation. APC acts in this complex by directing phosphorylated β-Catenin into a ubiquitination-mediated proteosomal degradation pathway. In the presence of Wnt, GSK-3β is inactivated, leaving an unphosphorylated, but active, β-Catenin. In addition to β-Catenin, axin also seems to be a target of GSK-3β. In the absence of Wnt stimulation, GSK-3β constitutively phosphorylates axin. In this phosphorylated state, axin contributes to the phosphorylation of β-Catenin by GSK-3β. When Wnts induce GSK-3β inactivation, axin is no longer phosphorylated by GSK-3β and becomes unstable in β-Catenin accumulation and gene activation.

Traditionally, the proximal signaling molecule in the canonical pathway is Dishevelled (Dsh). Dsh has been pictured to lie between membrane-bound receptors and the β-Catenin complex. Drosophila Dsh is a three-domain, 623-aa protein that is activated by Wnt binding to its seven-transmembrane (TM) receptor Frizzled. Following Wnt binding, PAR-1 or casein kinase II phosphorylates Dsh on its N-terminus. At this point, it becomes associated with the β-Catenin complex and blocks GSK-3β activity. This results in β-Catenin accumulation/stabilization and subsequent gene activation. However, Dsh phosphorylation may be a general phenomenon of Wnt activation, with the “true” canonical signal transmitted through the LRP.

Two non-canonical pathways are associated with Wnt signaling. The first is the Wnt/Ca²⁺ pathway. In this pathway, Wnt binding to a seven-TM Frizzled receptor results in the activation of heterotrimeric G-proteins with subsequent mobilization of phospholipase C and phosphodiesterase. This results in a decrease in cGMP, an increase in intracellular Ca²⁺, and activation of protein kinase C.

The second non-canonical pathway is the planar cell polarity (PCP) pathway. Activation of this pathway defines polarity in select epithelial tissues, particularly along an axis perpendicular to the apical-basal border. In general, Wnt binding to Frizzled activates Dsh, likely on the C-terminus of the molecule. Dsh then recruits RhoA/Rac, which ultimately leads to JNK (c-jun NH2-terminal kinase) pathway activation. A major target of the JNK pathway is the AP-1 (activator protein-1) transcription factor.

3. Frizzled Receptors

To date, there are 10 human Frizzled receptors. Frizzled receptors vary in length from 537 to 706 aa. All are seven-TM receptors with an extracellular N-terminus and an intracellular C-terminus. The Frizzled N-terminus is characterized by the presence of an α-helical, approximately 130-aa, cysteine-rich domain (CRD) that interacts with both Wnts and other Wnt receptors. The C-terminus has a membrane-proximal Lys-Thr-x-x-x-Trp motif (SED ID NO:149) that is associated with Dsh binding and activation. Phylogenetically, the Frizzled receptors fall into four groups. Frizzled-1, 2 and 7, and Frizzled-3 and 6 make up two related groups, while Frizzled-5 and 8 comprise a third group, and Frizzled-4, 9 and 10 generate a distant fourth group.

Frizzled-5 in human is 585 aa in size. There is a 26-aa signal sequence, a 212-aa N-terminus, and a 64-aa C-terminus. The N-terminus has two potential N-linked glycosylation sites and a 123-aa CRD. The C-terminus contains the typical Lys-Thr-x-x-x-Trp (SED ID NO:149) and Thr-Thr-Val motifs. In the fifth TM segment there is a 9-aa leucine zipper. Wnts known to act through Frizzled-5 include Wnt-7a, Wnt-5a, Wnt-10b, Wnt-2, Wnt-8 and Wnt-11.

Human Frizzled-7 is a 574-aa precursor with a 32-aa signal sequence, a 224-aa extracellular N-terminus, seven TM segments, and a 25-aa C-terminus that contains a Lys-Thr-x-x-x-Trp motif (SED ID NO:149) with a Thr-x-Val PDZ-binding tripeptide. Wnts reported to bind to Frizzled-7 include Wnt-5a, Wnt-8, and Wnt-11.

Human Frizzled-8 is quite long at 694 aa in length. It possesses a 27-aa signal sequence, a 248-aa extracellular N-terminus, and an 89-aa C-terminus. The N-terminus is somewhat unusual in that there are two potential N-linked glycosylation sites plus a polyproline segment and a polyglycine segment. The usual CRD also appears with ten cysteines embedded in a 120-aa segment. The C-terminus has a Thr-x-Val tripeptide, a Lys-Thr-x-x-x-Trp motif (SED ID NO:149), and a polyglycine repeat of 25 aa. Wnt-8 is believed to be a ligand for Frizzled-8.

Wnts reported to activate Frizzled-1 include Wnt-1, Wnt-8, Wnt-3a, Wnt-3, and Wnt-2; Wnt-5a apparently interacts with Frizzled-1, but does not signal. The only reported Wnt to interact with Frizzled-2 is Wnt-5a. Wnt-1 and Wnt-8 are reported to bind to Frizzled-3. Wnts believed to bind to Frizzled-4 include Xenopus Wnt-1, Wnt-8, and Wnt-5a. Frizzled-6 may be a primary target of the Wnt-modulating, secreted Frizzled-related proteins (sFRPs). Wnt-2 is reportedly a ligand for Frizzled-9. Wnt-8 binds to Frizzled-10.

4. LRPs (Low Density Lipoprotein Receptor-Related Proteins)

The two principal Wnt-related receptors in this group belong to the low density lipoprotein receptor (LDLR) gene family. In mammals, there are at least 10 members, five of which bind ApoE (Apolipoprotein E; LDL-R, VLDL-R, LRP-8, LRP-1, and LRP-2), and five that do not (LRP-4, LRP-5, LRP-6, LRP-3, and LR11). ApoE is part of the protein coat of chylomicrons, chylomicron remnants, VLDL (very low density lipoprotein) particles, and IDL (intermediate density lipoprotein) particles. The two LRP-family, Wnt-associated receptors are LRP-5 and LRP-6. Although they are often described as being only structural relatives of the LDL receptor, there is evidence that they, too, may play a role in lipid metabolism. First, LRP-5 is reported to bind ApoE. Second, LRP-5 is also reported to be essential for normal cholesterol and glucose metabolism. LRP-5−/− mice develop increased plasma cholesterol due to decreased hepatic clearance of remnant chylomicrons. In addition, low ApoE levels induce LRP-5 to participate in the clearance of dietary TGs. This effect may be indirect, however. Dickkopf-1 (Dkk-1) is a soluble modulator of Wnt-LRP activity. Structurally, Dkk-1 has two characteristic CRDs, and it is known to bind to LRP-5. One of the CRDs is reminiscent of colipase, which binds liver lipase and/or lipoprotein lipase. This binding results in lipoprotein lipase activation and subsequent lipid hydrolysis. Thus, it is possible that a LRP-5 complexed to Dkk-1 may contribute to lipase activity and TG hydrolysis.

As mediators of Wnt activity, LRP-5 and LRP-6 are presumed to function as coreceptors for Wnt signaling. Select Frizzled receptors, as well as LRP-5 and 6, are associated with signaling through the β-Catenin pathway. β-Catenin accumulation/activation/stabilization may be accomplished either via axin or Dsh. Activation of Frizzled results in Dsh activation, while activation of LRP results in axin destabilization. Both events impact the status of β-Catenin. On the cell surface, LRP-5 and 6 are normally inactive, and exist as heterodimers, homodimers, or oligomers via interactions between their extracellular EGF/Tyr-Trp-Thr-Asp (YWTD) repeats (SED ID NO:150). When Frizzled and LRP are brought together, Frizzled appears to separate the “associating” intracellular domains of multimer LRP, resulting in the exposure of a cytoplasmic [Pro]-Pro-Pro-Ser-Pro (PPSP) signaling motif (SED ID NO:151). Wnt in this complex perhaps serves as a bridge for Frizzled and LRP, or induces some type of conformational change in LRP. An exposed LRP cytoplasmic PPSP site is phosphorylated, creating a docking site for axin that leads to axin destabilization.

Human LRP-5 is a 175 to 180-kDa, type I, single-pass TM glycoprotein that is synthesized as a 1615-aa precursor. The precursor contains a 24-aa signal sequence, a 1361-aa extracellular region, a 23-aa TM domain and a 207-aa cytoplasmic tail. The extracellular region is complex. There are four, 40 to 50-aa EGF-repeats. Each EGF repeat contains six cysteines and an Arg-Gly-Gly-(Cys) motif (SED ID NO:152) at its N-terminus. An approximately 250-aa “spacer” separates each EGF repeat, and in each of these spacers lies a propeller-like structure composed of six blades. Five of the propeller blades contain an unusual and variable Tyr/Phe-Trp-Ile/Gly/Thr-Asp/Cys/Asn (YWTD) tetrapeptide (SED ID NO:153) in its structure. Distal to the EGF region lie three LDL-R repeats. A typical LDL-R repeat is approximately 40 aa long. In LRP-5, the three LDL-R repeats are 35 to 50 aa in length. There are six cysteines with a Ser-Asp-Glu tripeptide at the C-terminus of each repeat. It is thought that EGF repeats direct ligand dissociation, while LDL-R repeats are necessary for proper ligand binding. In general, the number of LDL-R repeats correlates with the number of different ligands that can be bound by a receptor. Although LRP-5 is a member of the LDL-R gene family, its EGF and LDL-R modules appear in reverse order relative to those on the receptor that normally binds LDL.

Human LRP-6 is synthesized as a 1613-aa precursor that contains a 19-aa signal sequence, a 1353-aa extracellular domain, a 23-aa TM segment, and a 218-aa cytoplasmic tail. As with LRP-5, there are four EGF repeats with intervening propeller structures that are followed by three LDL-R repeats. Studies reveal that EGF repeats 1 and 2 interact with Frizzled receptors, while repeats 3 and 4 interact with Dkk proteins. The cytoplasmic region is known to contain multiple PPSP motifs, the most membrane-proximal of which is absolutely required for Wnt signaling. Multimers of LRP-6 are inactive, while monomers and mutants missing the extracellular domain are constitutively active. LRP-6 is ubiquitously expressed. This allows it to serve as a β-Catenin pathway signal transducing receptor for a variety of Wnt/Frizzled pairs. Those included are Wnt-8/Frizzled-5, Wnt-11/Frizzled-5, Wnt-5a/Frizzled-5, Wnt-8/Frizzled-4, and Wnt-8/Frizzled-7.

5. Single Nucleotide Polymorphisms (SNP)

The disclosed method generally comprises detecting a single nucleotide polymorphism at nucleotide position 11987669 on chromosome 11. Specifically, a cystidine rather than a guanine at this position is an indication of increased risk of T2DM as disclosed herein.

A Single Nucleotide Polymorphism (SNP) is a DNA sequence variation occurring when a single nucleotide—A, T, C, or G—in the genome (or other shared sequence) differs between members of a species (or between paired chromosomes in an individual). SNPs may fall within coding sequences of genes, noncoding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are non-synonymous. SNPs that are not in protein coding regions may still have consequences for gene splicing, transcription factor binding, or the sequence of non-coding RNA.

6. SNP Detection Methods

A wide variety of techniques have been developed for SNP detection and analysis, see, e.g. Sapolsky et al. (1999) U.S. Pat. No. 5,858,659; Shuber (1997) U.S. Pat. No. 5,633,134; Dahlberg (1998) U.S. Pat. No. 5,719,028; Murigneux (1998) WO98/30717; Shuber (1997) WO97/10366; Murphy et al. (1998) WO98/44157; Lander et al. (1998) WO98/20165; Goelet et al. (1995) WO95/12607 and Cronin et al. (1998) WO98/30883. In addition, ligase based methods are described by Barany et al. (1997) WO97/31256 and Chen et al. Genome Res. 1998; 8(5):549-56; mass-spectroscopy-based methods by Monforte (1998) WO98/12355, Turano et al. (1998) WO98/14616 and Ross et al. (1997) Anal Chem. 15, 4197-202; PCR-based methods by Hauser, et al. (1998) Plant J. 16, 117-25; exonuclease-based methods by Mundy U.S. Pat. No. 4,656,127; dideoxynucleotide-based methods by Cohen et al. WO91/02087; Genetic Bit Analysis or GBA™ by Goelet et al. WO92/15712; Oligonucleotide Ligation Assays or OLAs by Landegren et al. (1988) Science 241:1077-1080 and Nickerson et al. (1990) Proc. Natl. Acad. Sci. (U.S.A.) 87:8923-8927; and primer-guided nucleotide incorporation procedures by Prezant et al. (1992) Hum. Mutat. 1:159-164; Ugozzoli et al. (1992) GATA 9:107-112; Nyreen et al. (1993) Anal. Biochem. 208:171-175, which are all hereby incorporated herein by reference for the teaching of SNP detection methods.

The disclosed method contemplates the use of any discovered method of detecting the disclosed SNP. For example, the method can comprise the use of restriction fragment length polymorphism; allele specific hybridization; TaqMan®, Molecular Beacon, or Scorpion® assay; allele specific oligonucleotide ligation; invader method; rolling circle DNA amplification; mass spectroscopy; gene sequencing, or variations thereof.

i. Allele Specific Hybridization

The provided method can comprise detecting the SNP by Allele Specific Hybridization. This method relies on selective hybridization to distinguish between two DNA molecules differing by one base. In general, the method involves applying labeled PCR fragments to immobilized oligonucleotides representing SNP sequences. After stringent hybridization and washing conditions, label intensity is measured for each SNP oligonucleotide.

Thus, the provided method can comprise providing a nucleic acid probe that hybridizes under stringent conditions to an oligonucleotide consisting of SEQ ID NO:1 but does not hybridize under stringent conditions to an oligonucleotide consisting of SEQ ID NO:2, and detecting hybridization of said probe to the nucleic acid sample. The nucleic acid probe can comprise at least about 5, 6, 7, 8, 9, 10, 11, 12, 13, 14, 15, 16, 17, 18, 19, 20, 21, 22, 23, 24, 25, 26, 27, 28, 29, or 30 nucleotides.

The probe can comprise a label such as a fluorescent dye (also known herein as fluorochromes and fluorophores). Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosanilin (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodamineIsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

ii. Single-Step Homogeneous Methods

TaqMan®, molecular beacon, and Scorpion® assay are all microtiter plate-based fluorescent readout systems, initially designed for real time PCR expression analyses. TaqMan® and molecular beacon both rely on allele-specific hybridization of oligonucleotides during PCR for allele discrimination, while scorpion assay can use either allele-specific PCR or allele-specific hybridization chemistry for allelic discrimination. They all can be performed as an endpoint assay in a completely homogeneous reaction. All the reagents and genomic DNA are mixed at the beginning, and the fluorescent signal is read after the thermocycling step. There is no separate pre-amplification step, or intermediate processing, making them the simplest assay formats possible.

Thus, the provided method can comprise detecting the SNP using TaqMan®. Allelic discrimination using this chemistry is based on the design of two TaqMan® probes, specific for the wildtype allele and the mutant allele. TaqMan® SNP analysis utilizes the 5′ exonuclease activity of DNA Taq polymerase and the quenching effects of specific florescent dyes to determine the relative frequency of each allele within an individual genome. Primers are designed against a conserved region of the genome flanking the locus of interest. Two probes are designed across the locus of interest, one for each allele. Each probe is labeled with a different reporter dye as well as a quencher molecule. Proximity to the quencher dye inhibits the florescence of the reporter molecule. During thermocycling, the probe anneals to the locus of interest in an allele specific manner. As the Taq DNA polymerase extends the primers, it also degrades the annealed probe, allowing the florescent dye to come out of the sphere of influence of the quencher and thus become detectable.

The provided method can comprise detecting the SNP using molecular beacons. Molecular beacons are oligonucleotide probes that can report the presence of specific nucleic acids in homogenous solutions (Tyagi S, Kramer F R. Molecular beacons: probes that fluoresce upon hybridization, Nature Biotechnology 1996; 14: 303-308.) Molecular beacons are hairpin shaped molecules with an internally quenched fluorophore whose fluorescence is restored when they bind to a target nucleic acid. They are designed in such a way that the loop portion of the molecule is a probe sequence complementary to a target nucleic acid molecule. The stem is formed by the annealing of complementary arm sequences on the ends of the probe sequence. A fluorescent moiety is attached to the end of one arm and a quenching moiety is attached to the end of the other arm. The stem keeps these two moieties in close proximity to each other, causing the fluorescence of the fluorophore to be quenched by energy transfer. Since the quencher moiety is a non-fluorescent chromophore and emits the energy that it receives from the fluorophore as heat, the probe is unable to fluoresce. When the probe encounters a target molecule, it forms a hybrid that is longer and more stable than the stem and its rigidity and length preclude the simultaneous existence of the stem hybrid. Thus, the molecular beacon undergoes a spontaneous conformational reorganization that forces the stem apart, and causes the fluorophore and the quencher to move away from each other, leading to the restoration of fluorescence.

The provided method can comprise detecting the SNP using Scorpion® primers. Scorpion® primers are bi-functional molecules in which a primer is covalently linked to the probe. The molecules also contain a fluorophore and a quencher. In the absence of the target, the quencher nearly absorbs the fluorescence emitted by the fluorophore. During the Scorpion® PCR reaction, in the presence of the target, the fluorophore and the quencher separate which leads to an increase in the fluorescence emitted. The fluorescence can be detected and measured in the reaction tube. The Scorpion® primer carries a Scorpion® probe element at the 5′ end. The probe is a self-complementary stem sequence with a fluorophore at one end and a quencher at the other. The Scorpion® primer sequence is modified at the 5′ end. It contains a PCR blocker at the start of the hairpin loop (Usually HEG monomers are added as blocking agent). In the initial PCR cycles, the primer hybridizes to the target and extension occurs due to the action of polymerase. Scorpion® primers can be used to examine and identify point mutations by using multiple probes. Each probe can be tagged with a different fluorophore to produce different colors. In Scorpion® primers, the probe is physically coupled to the primer which means that the reaction leading to signal generation is a unimolecular one. This is in contrast to the bi-molecular collisions required by other technologies such as TaqMan® or Molecular Beacons. After one cycle of PCR extension completes, the newly synthesized target region will be attached to the same strand as the probe. Following the second cycle of denaturation and annealing, the probe and the target hybridize. The denaturation of the hairpin loop requires less energy than the new DNA duplex produced. Consequently, the hairpin sequence hybridizes to a part of the newly produced PCR product. This results in the separation of the fluorophore from the quencher and causes emission.

The provided method can comprise detecting the SNP using an allele-specific amplification primers that have secondary priming sites for universal energy-transfer-labeled primers.

The provided method can comprise detecting the SNP using an AlphaScreen proximity assay. AlphaScreen generates an amplified light signal when donor and acceptor beads are brought to proximity, and this detection method can be combined with allele-specific amplification chemistry or allele-specific hybridization chemistry for allele discrimination.

iii. Allele Specific Oligonucleotide Ligation

The provided method can comprise detecting the SNP by Allele Specific Oligonucleotide Ligation. By designing oligonucleotides complementary to the target sequence, with the allele-specific base at its 3′-end or 5-′end, one can determine the genotype of the PCR amplified target sequence by determining whether an oligonucleotide complementary to the DNA sequencing adjoining the polymorphic site is ligated to the allele-specific oligonucleotide or not.

iv. Invader Method

There have been a few notable efforts to establish PCR-free genotyping methods. One such attempt is the Invader method (Third Wave Technologies), based on a matched nucleotide-specific cleavage by a structure-specific ‘flap’ endonuclease, in the presence of an invading oligonucleotide. The combination of this reaction with a secondary reaction using fluorescence resonance energy transfer (FRET) oligonucleotide cassettes, generates a highly allele-specific signal, in a completely homogeneous and isothermal reaction. In addition, the Invader assay's great sensitivity and excellent signal to noise ratio allow direct genotyping of genomic DNA samples without PCR. However, the amount of DNA currently required for reliable genotyping is high (50 ng range) for the analysis of a large number of SNPs. The Invader method can be combined with PCR to reduce the DNA requirement, which also makes the signal more robust.

v. Rolling Circle DNA Amplification

Another type of PCR-free genotyping is available through the combination of padlock probe ligation, and signal amplification by the rolling circle DNA amplification (RCA) process. In this assay, allele discrimination is accomplished by the specific ligation of completely matched oligonucleotides, in the same way as oligonucleotide ligation assay (OLA). The difference here is that the ligation of a padlock probe creates a circular DNA, which can be amplified by rolling circle DNA synthesis by a DNA polymerase. The high degree of signal amplification by rolling circle synthesis and the specificity of the allele-discrimination by DNA ligase, make padlock probe/RCA assay sensitive enough to be directly applied to genomic DNA. However, typical padlock probe/RCA genotyping still requires a large quantity of DNA (100 ng) per genotype, again making it less than ideal for the analysis of many SNPs. However, FRET primers (Amplifluor) can be used for signal detection in reducing the DNA requirement to a nanogram level.

vi. Mass Spectroscopy

The provided method can comprise detecting the SNP by mass spectroscopy. The principle of this method is to use mass spectrometry to detect the product of enzymatic allele-discrimination reaction directly or indirectly. Various allele discrimination chemistries such as single-base extension and its variation, allele-specific hybridization of peptide nucleic acid (PNA), Invader, and allele-specific PCR, have all been successfully combined with the mass spectrometry detection. Combinations of single-base extension or its modifications with matrix-assisted laser desorption/ionization time-of-flight (MALDI-TOF) mass spectrometry are the most commonly used, and have been made into commercial products by companies such as Sequenom and Applied Biosystems/PerSeptive Biosystems. The advantage of the MALDI-TOF mass spectrometry-based detection is in its speed and multiplexing capability. For example, a moderate mass spectrometer capable of recording 40,000 spectra a day, can theoretically score 200,000 genotypes in a 5-plex detection format. However, their rate limiting steps are generally not in the detection process by a mass spectrometer, but are in the preceding enzymatic reactions, and post-reaction sample processing steps. In most mass spectrometry-based assays, 5-plex may be the realistic limit for multiplexing to get reliable signals, partly due to the limitations in the detectable mass range and in the sensitivity of mass discrimination. Post-reaction sample processing is more complicated than that of most other genotyping formats, as a very high purity is necessary for the samples to be analyzed by a mass spectrometer. Solid phase sample processing with ion-exchange resin is employed in Sequenom's MassArray automated system, while miniaturized reverse phase liquid chromatography is used for Applied Biosystems/PerSeptive Biosystem's product to address this issue. Another system called ‘GOOD assay’ involves a use of chemically modified primers in the reaction, followed by an enzymatic removal of unextended primers and alkylation of the product, allowing a simplified and effective sample preparation for mass spectrometry.

Genotype accuracy due to the intrinsic nature of mass spectrometry is another advantage. The sensitivity of the instrument, the mass specificity of each reaction product, and for some type of reactions the fact that each reaction contains internal standards for calibration, all contribute to this accuracy. Mass spectrometry-based methods give little background especially when detecting the allelic discrimination reaction products directly, allowing accurate and automated genotype calling.

A different mass spectrometry-based assay has been made into a commercial product as Qiagen's MassCode system. This assay combines allele-specific PCR with UV-cleavable ‘mass tags’, and mass spectrometry detection. Here, mass spectrometry detects the cleaved tags and not the extension products themselves. Use of these ‘mass tags’ makes highly-multiplexed detection by a relatively simple mass spectrometer possible. One the other hand, this method can be more prone to background signal at least theoretically, as the mass spectrometer does not directly detect the allele-discrimination reaction product. For example, incomplete removal of free ‘mass tag’ labeled primers before UV-cleavage can cause a false signal in this method.

Matrix-assisted laser desorption/ionization (MALDI) is a soft ionization technique used in mass spectrometry, allowing, among other things, the ionization of biomolecules (biopolymers such as proteins, peptides and sugars) which tend to be more fragile and quickly lose structure when ionized by more conventional ionization methods. A matrix is used to protect the biomolecule from being destroyed by direct laser beam and to facilitate vaporization and ionization. The matrix consists of crystallized molecules, of which the three most commonly used are 3,5-dimethoxy-4-hydroxycinnamic acid (sinapinic acid), α-cyano-4-hydroxycinnamic acid (alpha-cyano or alpha-matrix) and 2,5-dihydroxybenzoic acid (DHB). A solution of one of these molecules is made, often in a mixture of highly purified water and an organic solvent (normally acetonitrile (ACN) or ethanol). Trifluoroacetic acid (TFA) may also be added. A good example of a matrix-solution would be 20 mg/mL sinapinic acid in ACN:water:TFA (50:50:0.1). The matrix solution is generally mixed with the analyte (e.g. protein-sample). The organic solvent allows hydrophobic molecules to dissolve into the solution, while the water allows for water-soluble (hydrophilic) molecules to do the same. This solution is spotted onto a MALDI plate (usually a metal plate designed for this purpose). The solvents vaporize, leaving only the recrystallized matrix, but now with analyte molecules spread throughout the crystals. The matrix and the analyte are said to be co-crystallized in a MALDI spot. The laser is fired at the crystals in the MALDI spot. The spot absorbs the laser energy and it is thought that primarily the matrix is ionized by this event. The matrix is then thought to transfer part of its charge to the analyte molecules (e.g. protein), thus ionizing them while still protecting them from the disruptive energy of the laser. Ions observed after this process are quasimolecular ions that are ionized by the addition of a proton to [M+H]+, or other cation such as sodium ion [M+Na]+, or the removal of a proton [M−H]− for example. MALDI generally produces singly-charged ions, but multiply charged ions ([M+nH]n+) can also be observed, usually in function of the matrix used and/or of the laser intensity, voltage.

vii. Sequencing

The provided method can comprise detecting the SNP by gene sequencing. Sequencing is the procedure of choice for SNP discovery. The most common forms of sequencing are based on primer extension using either a) dye-primers and unlabeled terminators or b) unlabeled primers and dye-terminators. The products of the reaction are then separated using electrophoresis using either capillary electrophoresis or slab gels.

Pyrosequencing employs an elegant cascade of enzymatic reactions to detect nucleotide incorporation during DNA synthesis. When a nucleotide is incorporated at the 3′-end by DNA polymerase, a pyrophosphate is released that is immediately converted to ATP by ATP sulfurylase. This ATP causes the oxidization of luciferin by luciferase, which is detected as a light signal. Pyrosequencing was initially developed as a DNA sequencing method, with a chemistry completely different from the Sanger dideoxynucleotide method. It is also a unique homogeneous sequencing method with no electrophoresis. Its capability to read flanking sequences as well as the SNP position itself, and its high specificity (ie non-specific binding will not generate a false signal) make it an accurate and attractive SNP genotyping method. In this method, alleles can be called by analyzing the individual sample itself, without comparing its signal to that of other samples or controls. This makes Pyrosequencing suitable for fully automated genotype calling, an important component of high throughput analyses. A 96-well medium throughput machine and a fully automated 384-well format high-throughput machine, are available from Pyrosequencing AB (Uppsala, Sweden) for this method, and the latter has capacity to score high thousands to low tens of thousands of genotypes a day. Pyrosequencing can be done in a duplex or a triplex format at least for some SNP combinations.

7. Primers and Probes

Thus, disclosed are compositions including primers and probes, which are capable of interacting with the disclosed nucleic acids, such as those comprising the SNP disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

8. Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

### C. Methods of Monitoring and Treatment

Also provided is a method of monitoring a subject identified as at increased risk for developing type 2 diabetes by the methods disclosed herein. As disclosed herein, the disclosed SNP is located 965 by upstream of the dkk3 transcription start site and affects gene transcription. Further as disclosed herein, the decreased levels of DKK3 resulting from the SNP are involved in the development of T2DM. Thus, for example, the subject can be monitored for levels of DKK3 in a tissue or bodily fluid. For example, the method can comprise obtaining a tissue or bodily fluid from a subject identified as having the disclosed SNP and measuring DKK3 levels in the bodily fluid. The bodily fluid can be, for example, blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid. The tissue can be any tissue that suffers microvascular complications associated with diabetes. Thus, the tissue can be obtained from, for example, the kidney or eye.

For example, levels of DKK3 can be measured at the mRNA level by real-time RT-PCR or at the level of the protein by extracting protein from the above-mentioned fluid(s) or tissue(s), resolving protein fragments in a denaturing gel and detecting and quantifying the levels of DKK3 using an antibody against the protein. However, other methods are known in the art and can be used in the disclosed methods.

It is understood that any method for the detection and measurement of DKK3 levels can be used in the disclosed method. For example, an immunodetection method can be used. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

### D. Methods of Treatment

Also disclosed herein is a method, comprising administering a therapeutically effective amount of an agent that increases DKK3 activity to a subject identified as having T2DM or the disclosed SNP. Also disclosed herein is a method, comprising administering a therapeutically effective amount of an agent that activates the Wnt pathway to a subject identified as having having T2DM or the disclosed SNP. The agent of the disclosed method can be an agonist of an activity of DKK3. “Activities” of a protein include, for example, transcription, translation, intracellular translocation, secretion, phosphorylation by kinases, cleavage by proteases, homophilic and heterophilic binding to other proteins, or ubiquitination. For example, DKK3 activity can include binding to Wnt-related receptors, such as a Frizzled (Fz or FZD) receptor, LRP5 or LRP6. Thus, DKK3 activity can include modulation of Wnt pathway. DKK3 activity can include modulation of β-catennin dependent Wnt signaling. DKK3 activity can include modulation of β-catennin independent Wnt signaling. For example, DKK3 activity can include modulation of planar cell polarity (PCP) and/or the Wnt/Ca²⁺ cascade. Thus, the agent of the disclosed method can be a small molecules, antibody, intracellular signaling molecule that binds and activates one or more of these targets. The agent of the disclosed method can in some aspects comprise a mimetic of DKK3 or a receptor thereof disclosed herein.

1. DKK3 Peptide

For example, the agent of the disclosed method can in some aspects be a peptide comprising DKK3 or a fragment thereof. An amino acid sequence for full length human DKK3 is set forth in SEQ ID NO: 160. A nucleotide sequence for full length human DKK3 is set forth in SEQ ID NO: 158, the corresponding coding sequence of which is set forth in SEQ ID NO: 159. Thus, the agent of the disclosed method can be a polypeptide comprising SEQ ID NO:160 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can bind a Wnt-related receptor, such as a Frizzled (Fz) receptor, LRP5 or LRP6, and modulate the Wnt pathway. The agent can in some aspects comprise a nucleic acid encoding DKK3 or a fragment thereof. Thus, the agent of the disclosed method can be a nucleic acid comprising SEQ ID NO:159 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can bind a Wnt-related receptor, such as a Frizzled (Fz) receptor, LRP5 or LRP6, and modulate the Wnt pathway.

The agent can in some aspects comprise an activator of Wnt signaling. Thus, the agent can in some aspects comprise an FZ5 agonist. An amino acid sequence for full length human FZ5 is set forth in SEQ ID NO: 163. A nucleotide sequence for full length human FZ5 is set forth in SEQ ID NO: 161, the corresponding coding sequence of which is set forth in SEQ ID NO: 162. Thus, the agent of the disclosed method can be a polypeptide comprising SEQ ID NO:163 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ5 or a fragment thereof. Thus, the agent of the disclosed method can be a nucleic acid comprising SEQ ID NO:163 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human FZ7 is set forth in SEQ ID NO: 184. A nucleotide sequence for full length human FZ7 is set forth in SEQ ID NO: 182, the corresponding coding sequence of which is set forth in SEQ ID NO: 183. The agent can in some aspects comprise an FZ7 agonist. Thus, the agent of the disclosed method can be a polypeptide comprising SEQ ID NO:184 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ7 or a fragment thereof. Thus, the agent of the disclosed method can be a nucleic acid comprising SEQ ID NO:183 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human FZ8 is set forth in SEQ ID NO: 166. A nucleotide sequence for full length human FZ8 is set forth in SEQ ID NO: 164, the corresponding coding sequence of which is set forth in SEQ ID NO: 165. The agent can in some aspects comprise an FZ8 agonist. Thus, the agent of the disclosed method can be a polypeptide comprising SEQ ID NO:166 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ7 or a fragment thereof. Thus, the agent of the disclosed method can be a nucleic acid comprising SEQ ID NO:165 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human LRP6 is set forth in SEQ ID NO: 172. A nucleotide sequence for full length human LRP6 is set forth in SEQ ID NO: 170, the corresponding coding sequence of which is set forth in SEQ ID NO: 171. Thus, the agent can in some aspects comprise an LRP6 agonist. Thus, the agent of the disclosed method can be a polypeptide comprising SEQ ID NO:172 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding LRP6 or a fragment thereof. Thus, the agent of the disclosed method can be a nucleic acid comprising SEQ ID NO:171 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human LRP5 is set forth in SEQ ID NO: 169. A nucleotide sequence for full length human LRP5 is set forth in SEQ ID NO: 167, the corresponding coding sequence of which is set forth in SEQ ID NO: 168. Thus, the agent can in some aspects comprise an LRP5 agonist. Thus, the agent of the disclosed method can be a polypeptide comprising SEQ ID NO:169 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding LRP5 or a fragment thereof. Thus, the agent of the disclosed method can be a nucleic acid comprising SEQ ID NO:168 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human FZ1 is set forth in SEQ ID NO: 175. A nucleotide sequence for full length human FZ1 is set forth in SEQ ID NO: 173, the corresponding coding sequence of which is set forth in SEQ ID NO: 174. Thus, the agent can in some aspects comprise an FZ1 agonist. Thus, the agent of the disclosed method can be a polypeptide comprising SEQ ID NO:175 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ1 or a fragment thereof. Thus, the agent of the disclosed method can be a nucleic acid comprising SEQ ID NO:174 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human FZ2 is set forth in SEQ ID NO: 178. A nucleotide sequence for full length human FZ2 is set forth in SEQ ID NO: 176, the corresponding coding sequence of which is set forth in SEQ ID NO: 177. Thus, the agent can in some aspects comprise an FZ2 agonist. Thus, the agent of the disclosed method can be a polypeptide comprising SEQ ID NO:178 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ2 or a fragment thereof. Thus, the agent of the disclosed method can be a nucleic acid comprising SEQ ID NO:177 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human FZ3 is set forth in SEQ ID NO: 181. A nucleotide sequence for full length human FZ3 is set forth in SEQ ID NO: 179, the corresponding coding sequence of which is set forth in SEQ ID NO: 180. Thus, the agent can in some aspects comprise an FZ3 agonist. Thus, the agent of the disclosed method can be a polypeptide comprising SEQ ID NO:181 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ3 or a fragment thereof. Thus, the agent of the disclosed method can be a nucleic acid comprising SEQ ID NO:180 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

An amino acid sequence for full length human FZ10 is set forth in SEQ ID NO: 187. A nucleotide sequence for full length human FZ10 is set forth in SEQ ID NO: 185, the corresponding coding sequence of which is set forth in SEQ ID NO: 186. Thus, the agent can in some aspects comprise an FZ10 agonist. Thus, the agent of the disclosed method can be a polypeptide comprising SEQ ID NO:187 or a fragment thereof of at least about 4, 5, 6, 7, 8, 9, 10, 15, 20, 25, 30, 35, 40, 45, 50, 55, 60, 65, 70, 75, 80, 85, 90, 100, 150, 200, 250, or 300 amino acids in length that can modulate the Wnt signaling pathway. The agent can in some aspects comprise a nucleic acid encoding FZ10 or a fragment thereof. Thus, the agent of the disclosed method can be a nucleic acid comprising SEQ ID NO:186 or a fragment thereof of at least about 30, 35, 40, 45, 50, 55, 60, 65, 70, 75, 80, 85, 90, 95, 100, 110, 120, 130, 140, 150, 160, 170, 180, 190, 200, 300, 400, 500, or 600 nucleic acids in length that encodes a peptide that can modulate the Wnt signaling pathway.

2. Pharmaceutical Carriers

The disclosed compositions can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, nicotinic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

3. Administration

A composition disclosed herein may be administered in a number of ways depending on whether local or systemic treatment is desired, and on the area to be treated. For example, the compositions may be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of the DKK3 agonist used alone might range from about 1 μg/kg to up to 100 mg/kg of body weight or more per day, depending on the factors mentioned above.

Following administration of a disclosed composition for treating, inhibiting, or preventing T2DM, the efficacy of the therapeutic can be assessed in various ways well known to the skilled practitioner. For instance, one of ordinary skill in the art will understand that a composition disclosed herein is efficacious in treating or inhibiting a T2DM in a subject by observing that the composition modulates levels of plasma glucose, fasting plasma, random plasma glucose, or glycosylated hemoglobin (Hb_{A1c}/A1C).

Efficacy of the administration of the disclosed composition may also be determined by measuring DKK3 levels or activity. DKK3 concentrations can be measured by methods that are known in the art, for example, using polymerase chain reaction assays to detect the presence of DKK3 mRNA or antibody assays to detect the presence of DKK3 protein in a sample (e.g., but not limited to, blood) from a subject or patient, or by measuring the level of circulating DKK3 levels in the patient. Efficacy of the administration of the disclosed composition may also be determined by measuring Wnt signaling activity. Efficacy of the administration of the disclosed composition may also be determined by measuring levels of TCF7L2. Efficacy of the administration of the disclosed composition may also be determined by measuring neovascularization in, for example, the retina of the subject.

The compositions that promote DKK3 may be administered prophylactically to patients or subjects who are at risk for microvascular complications.

The disclosed compositions and methods can also be used for example as tools to isolate and test new drug candidates for a variety of diabetes related diseases/complications.

### E. Method of Screening

A method of identifying a composition for treating or preventing type II diabetes mellitus, comprising contacting a candidate agent to a cell comprising a nucleic acid encoding DKK3 operably linked to an expression control sequence and detecting DKK3 levels and/or activity in the cell, wherein detection of an increase in DKK3 levels and/or activity in the cell compared to a reference control identifies a composition for treating or preventing type II diabetes mellitus.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the desired activity should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates DKK3 levels or activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of DKK3.

### F. Methods of Making the Compositions

The compositions disclosed herein and the compositions necessary to perform the disclosed methods can be made using any method known to those of skill in the art for that particular reagent or compound unless otherwise specifically noted.

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### G. Definitions

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a oligonucleotide” includes a plurality of such oligonucleotides, reference to “the oligonucleotide” is a reference to one or more oligonucleotides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “effective amount” refers to such amount as is capable of performing the function of the compound or property for which an effective amount is expressed. As will be pointed out below, the exact amount required will vary from process to process, depending on recognized variables such as the compounds employed and the processing conditions observed. Thus, it is not typically possible to specify an exact “effective amount.” However, an appropriate effective amount may be determined by one of ordinary skill in the art using only routine experimentation. In various aspects, an amount can be therapeutically effective; that is, effective to treat an existing disease or condition. In further various aspects, a preparation can be prophylactically effective; that is, effective for prevention of a disease or condition. In a further aspect, a compound or moiety can be provided in an amount effective to perform an imaging function.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Variant of DKK3 Gene Increases Risk of Type 2 Diabetes

i. Materials and Methods

Patients: All subjects provided informed consent prior to participation in the study. The T2DM patients were enrolled at Sichuan Provincial Hospital, Utah Diabetes Center, and the renal dialysis facilities of University of Utah. T2DM status was defined as meeting either the ADA and/or WHO criteria where the patient had symptoms of diabetes including polyuria, polydipsia, and unexplained weight loss and a fasting plasma glucose (FPG)≧126 mg/dl (7.0 mmol/l). Normal controls were defined as a fasting plasma glucose (FPG)<100 mg/dl (5.6 mmol/l) and no symptoms of diabetes. Patient characteristics of Chinese and Utah cohorts are listed in Table 1.

Genotyping: The initial T2DM Chinese cohort including 327 individuals from Southern China was genotyped and allele frequencies were compared to 215 age and ethnicity matched control patients. The Chinese cohort from northern China for second stage replication genotyping of rs11022111 included 274 T2DM patients, and 94 control patients. In addition, a third Utah cohort of 411 Caucasion T2DM patients and 150 age and ethnicity matched normal control patients was genotyped.

For SNP genotyping, genomic DNA extracted were PCR-amplified from case and control patient blood samples. SNPs were genotyped using either direct DNA sequencing or the SNAPSHOT method on an ABI 3130 genetic analyzer (Applied Biosystems, Foster City, Calif.) as previous described (Yang, Z., et al. 2006) and according to the manufacturer's instructions. All SNPs reported in this manuscript had a genotyping success rate >98% and accuracy >99% as judged by random re-genotyping of 10% samples.

Genotypes of rs11022111 in the Japanese cohort were determined by a PCR-invader assay. Briefly, amplification of the target was performed with 10 ng of genomic DNA in a 20 μl reaction containing 5 pmol of each primer, 5 units of ExTaq HS (Takara Bio, Otsu, Japan). Initial denaturation was at 94° C. for 2 min, followed by 35 cycles of denaturation at 94° C. for 15 s, annealing at 60° C. for 45 s, and extension at 72° C. for 3 min. The amplified products were diluted 10 times, and 2 μl were dried and used for the assay. Allele-specific oligonucleotide pairs and invasive probes were designed and supplied by Third Wave Technologies (Madison, Wis.). Each reaction volume of 3 μl contained 0.15 μl signal buffer, 0.15 μl FRET probes, 0.15 μl clevase, and 0.3 μl of probe-mix. Samples were incubated at 95° C. for 5 min, and then at 63° C. for 20 min in the ABI7700 (Applied Biosystems).

Fourty seven tagging SNPs were chosen in candidate genes involved in Wnt based on the International HapMap CEU data. A complete list of primers for each gene used in the SNP SNPSHOT assay is listed in Table 2.

Data analysis: All SNP genotyping results were screened for deviations from Hardy-Weinberg equilibrium (p<0.01) and SNPs with any significant deviation were excluded from analysis. The chi-squared test for trend for an additive model or dominant model over alleles was performed to assess evidence for association using PEPI version 4.0. Odds Ratios and 95% confidence intervals were calculated by conditional logistic regression using SPSS version 13.0 to estimate risk size for the heterozygotes and homozygotes for the risk alleles. For the risk genotypes identified, population attributable risks (PAR) were calculated using the Levin formula (Levin, M. L. 1953). LD structure was examined using Haploview (version 3.32). The default settings were used creating a 95% confidence bounds on D′ to define pair-wise SNP's in strong LD. Haploview was also used for allelic association tests and for multiple permutation testing calculations using 10,000 permutations for each SNP.

DKK3 immunohistochemstry: Mouse pancreas and adipocyte tissues were prepared as previously described (Yang, Z., et al. 2006). Cryosections were obtained after fixation with 4% paraformaldehyde on ice for 2 hours, and incubated in 15% and 30% sucrose overnight consequently. Immunohistochemistry was performed using 2 μg/ml rabbit polyclonal anti-DKK3 antibody (Santa Cruz Biotechnology, Santa Cruz, Calif.) and FITC conjugated goat anti-rabbit IgG antibody (Jackson Immunoresearch, West Grove, Pa.). Immunolabeling was visualized using a Zeiss LSM 510 laser scanning confocal microscope (Zeiss, Thornwood, N.Y.) at the same level of parameter.

RT-PCR of DKK3 mRNA in db/db mouse kidney: The db/db mice are among the best characterized and most intensively investigated mouse model for diabetes and diabetic kidney disease (Sharma, K., et al. 2003). Animals were obtained from Jackson Laboratory. Total RNA was isolated from 15 week db/db mice. The RNA was converted into cDNA (Invitrogen, SuperScrit™ III First-Strand Synthesise System for RT-PCR, Cat. No: 18080-051). 50 ng cDNA was used for real-time PCR (Qiagen, QuantiTect SYBR Green PCR Kit) with the following primers:

| Mus-DKK3-qPCR-L: | |
|---|---|
| ccaccctgctgcttttactc | (SEQ ID NO: 1) |
| and | |
| Mus-DKK3-qPCR-R: | |
| ctcagtctgggaccttctgc, | (SEQ ID NO: 2) |

for the mouse DKK3 gene to generate a 166 by product. GAPDH expression was used to normalize DKK3 expression using the following primers:

| Mus-GAPDH-QPCR-L2: | |
|---|---|
| gtgaaggtcggtgtgaacgg | (SEQ ID NO: 3) |
| and | |
| Mus-GAPDH-QPCR-R2: | |
| gccgttgaatttgccgtgag. | (SEQ ID NO: 4) |

The RT-PCR was performed simultaneously for DKK3 and GAPDH on an ABI 7300 real-time PCR system. RT-PCR conditions were one cycle of 50° C.-2 minutes, 95° C.-15 minutes, followed by 35 cycles in which each cycle included 94° C.-15 seconds, 58° C.-30 seconds and 72° C.-30 seconds.

Expression constructs and luciferase assays for DKK3 promoter SNP rs11022111: A DNA fragment containing −2 to −1500 bp from the DKK3 translation site including either C (rs11022111-C construct) or G allele (rs11022111-G construct) of rs11022111 at position −965 was PCR amplified from genomic DNA of diabetic patients using the following primers:

forward:
(SEQ ID NO: 5)
5′-gaagatcccgttggggtttcaagctggaga-3′
and
reverse:
(SEQ ID NO: 6)
5′-cccaagcttgggtccgctctgcgcccgcagc-3′.

These constructs were subcloned into the Bgl II-Hind III site of the pGL3-basic vector (Promega, Madison, Wis., USA) respectively. All constructs were verified by restriction digestion and bidirectional DNA sequencing. It was confirmed that the only difference between the rs11022111-C construct and rs1102211-G construct is C or G at rs11022111 and the rest of DKK3 promoter sequence is identical to that of native human DKK3 promoter sequence. HEK293 cells were split into 24-well plates and co-transfected 24 h later with 1 ng of the transfection control Renilla luciferase plasmid pTK-RL (Promega, Madison, Wis., USA) and 200 ng of the following plasmids: pGL3-control plasmid, rs11022111-C construct, rs11022111-G construct respectively. Transfections (n=12) were done using a Fugene-6 protocol according to the manufacture's specifications (Roche Applied Science, Mannheim, Germany). 48 h after transfection, cells were washed with PBS twice and luciferase activities were measured with Dual-Luciferase Assay Kit (Promega, Madison, Wis., USA). Fold induction was derived relative to normalized DKK3 activity.

Morpholinos and embryo manipulations: A translational morpholinos against dkk3 (5′-AGAGGCTGAATCCGAGCAGAAACAT-3′ (SEQ ID NO:8)) as well as a control morpholino (5′-CCTCTTACCTCAGTTACAATTTATA-3′ (SEQ ID NO:9)) were designed by and obtained from Gene Tools, LLC. 1 nl of diluted MO was injected into wild-type zebrafish embryos at the 1 to 2-cell stage. Injected embryos were observed for 24-30 h and scored. For RNA rescue experiments, dkk3 mRNA was transcribed in vitro using the SP6 mMessage mMachine kit (Ambion). Morphant embryos were classified into two graded phenotypes depending on the relative severity compared to age-matched controls from the same clutch by two independent investigators masked to the experimental details.

Classification of zebrafish embryos: The classes the embryos were grouped into were defined as follows: Class I) the embryos grouped into this class were mildly to moderately affected and displayed a shortened body axis, mediolaterally elongated somites, and notochord imperfections; Class II) embryos in this subset were more severely affected as defined by shortened body axes, bubbling of cells, mediolaterally elongated somites, and widened and kinked notochord.

Luciferase Reporter Assays: Experiments were carried out with HEK 293T cells (pTOPFlash/pFOPFlash) or HEK 293T cells stably expressing pTOPFlash reporter (pRK5 HA-Dkk3, pRK5 LRP5, pRK5 LRP6, pRK5 mFz1-Fz10, pSuper hsDKK3 541, pBSKS+Wnt1-Wnt 7b). Cells were seeded in 24 well plates at a density of 10⁴ cells per well. After 18-24 h, six wells each were transfected with reporter plasmid (Veeman, M. T., et al. 2003) and/or Renilla Luciferase cDNA in an SV40 vector and the plasmid of interest using the Polyfect (Qiagen) optimized transfection protocol. Plasmids used were Super8xTOPFlash (Veeman, M. T., et al. 2003) and pRL SV40 (renilla luciferase) as an internal control, pFOPFlash (Korinek, V., et al. 1997) to ensure TCF/LEF1 transcriptional specificity. If applicable, three wells each were treated with Wnt3a enriched medium after another 24 h, which was aspirated from Wnt3a/L cells (Willert, K., et al. 2003) and sterile filtered before applying it to the luciferase assay. Cells were lysed and luciferase activity measured 48 h after start of stimulation using the Promega Dual Luciferase Reporter Assay System (E1910) and a GeniosPro Luminometer (Tecan).

RT-PCR of TCF7L2 mRNA: 293T cells were transfected with combinations of pSuper hsDKK3 541, pRK5 DKK3, pRK5 mFz1-2, mFz5, mFz7-8 and respective control vectors using FuGene6 at 80% confluency. 48 h post-transfection, cells were stimulated with Wnt3a-conditioned medium and harvested after 24 h. Total RNA was isolated with Trizol (Invitrogen) and converted into cDNA using the SuperScript II First Strand kit (Invitrogen) according to manufacturer's specifications. qPCR was carried out in triplicate with SYBR GreenER (Invitrogen) and the following primers for TCF7L2:

| Fwd: | |
|---|---|
| 5′-CGTAGACCCCAAAACAGGAA-3′ | (SEQ ID NO: 10) |
| and | |
| Rev: | |
| 5′-TCCTGTCGTGATTGGGTACA-3′ | (SEQ ID NO: 11) |

on an ABI Prism 7500. The relative copy numbers calculated from Ct observed were normalized against β-actin or 18S.

RT-PCR of axin2 mRNA in zebrafish: Total RNA was isolated from individual embryos using Trizol (Invitrogen) according to manufacturer's specifications. cDNA was generated using the SuperScript II First Strand kit (Invitrogen) according to manufacturer's specifications. qPCR was carried out in triplicate with SYBR GreenER (Invitrogen) and the following primers for axin2:

Fwd:

| | |
|---|---|
| 5′-CGGATGACTCCATGTCAATG-3′ | (SEQ ID NO: 154) |
| and | |
| Rev: | |
| 5′-GGCTATCAACTGTGCTGCAA-3′ | (SEQ ID NO: 155) |

on an ABI Prism 7500. The relative copy numbers calculated from Ct observed were normalized against β-actin with the following primers:

Fwd:

| | |
|---|---|
| 5′-CCCTTGACTTTGAGCAGGAG-3′ | (SEQ ID NO: 156) |
| and | |
| Rev: | |
| 5′-ACAGGTCCTTACGGATGTCG-3′. | (SEQ ID NO: 157) |

ii. Results

Wnt involvement in T2DM and association with DKK: The Wnt signaling pathway plays a diverse role in development and homeostasis and Wnt dysregulation has been associated with a variety of phenotypes, Wnt/β-catenin activation is most prominently associated with neoplasia (Logan, C. Y. & Nusse, R. 2004). In the adult pancreas, the Wnt co-receptor LRP5 is required for normal cholesterol and glucose metabolism (Fujino, T., et al. 2003), whereas a subset of Wnt ligands are required for adipocyte differentiation and have been implicated in early onset obesity (Christodoulides, C., et al. 2006).

A potentially informative disorder with respect to sporadic T2DM is familial exudative vitreoretinopathy (FEVR), as it shares similarities with diabetic retinopathy, a common manifestation of T2DM. Genetic studies have shown FEVR to be caused by loss of function mutations in two Wnt co-receptors, Frizzled 4 (Fz4) and the low-density lipoprotein receptor-related protein 5 (LRP5) (Toomes, C., et al. 2004; Robitaille, J., et al. 2002; Hey, P. J., et al. 1998). Disruption of the same pathway has also been implicated in Norrie disease, another disorder of vascular development (Xu, Q., et al. 2004). Moreover, defective Wnt signaling has been documented in mice and zebrafish ablated for several proteins that cause Bardet-Biedl syndrome (Ross, A. J., et al. 2005), a genetically heterogeneous disorder characterized in part by insulin-resistant diabetes, truncal obesity and hypertension (Katsanis, N., et al. 2001). These data, together with the association of TCF7L2 with T2DM in several populations, and the observation that TCF7L2 mRNA levels are correlated with the incidence of impaired insulin secretion (Munoz, J., et al. 2006; Saxena, R., et al. 2006; Damcott, C., et al. 2006) and possibly insulin resistance (Damcott, C., et al. 2006) suggested that components in the Wnt signaling pathway might contribute to the genetic risk to T2DM.

Because metabolic factors including obesity, hypertension, hyperlipidemia, and hypercholesterolemia can influence development of T2DM, a Chinese cohort with a lower average body mass index (BMI, mean BMI<25) and lower blood pressure and lipid profiles was chosen for initially study so as to minimize the impact of these factors while trying to identify genes that may be specifically associated with T2DM (Table 1). To initially evaluate the Wnt signaling pathway association with the genetic risk of T2DM, a panel of 10 SNPs in 5 candidate genes involved in the Wnt pathway (either tagging SNPs, or SNPs of potentially functional relevance to each gene (Table 3) was designed and used to genotype 338 cases with T2DM from Southern China and 223 age and ethnicity matched control patients without T2DM or hypertension and mean BMI<25, (Table 1). No significant association was observed with Fz4, DKK1, DKK2, LRP5 (P>0.05, Table 3).

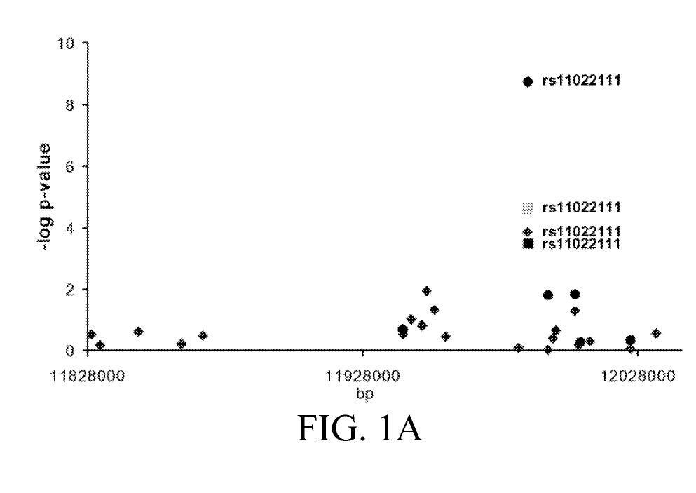

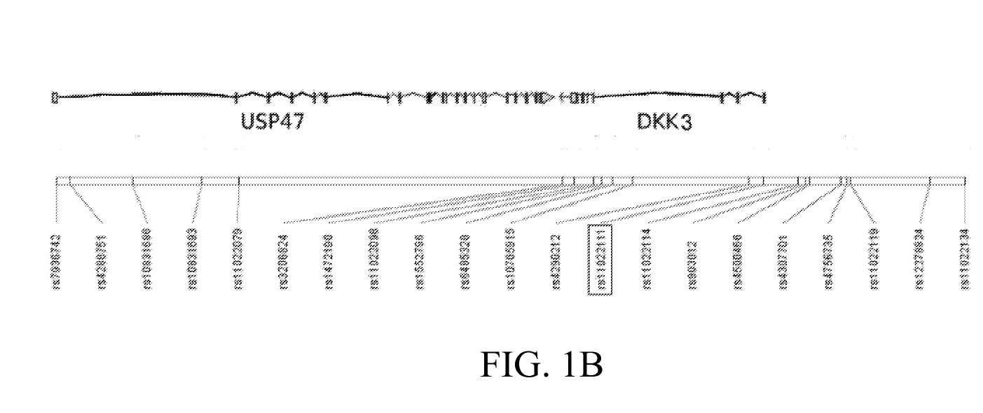

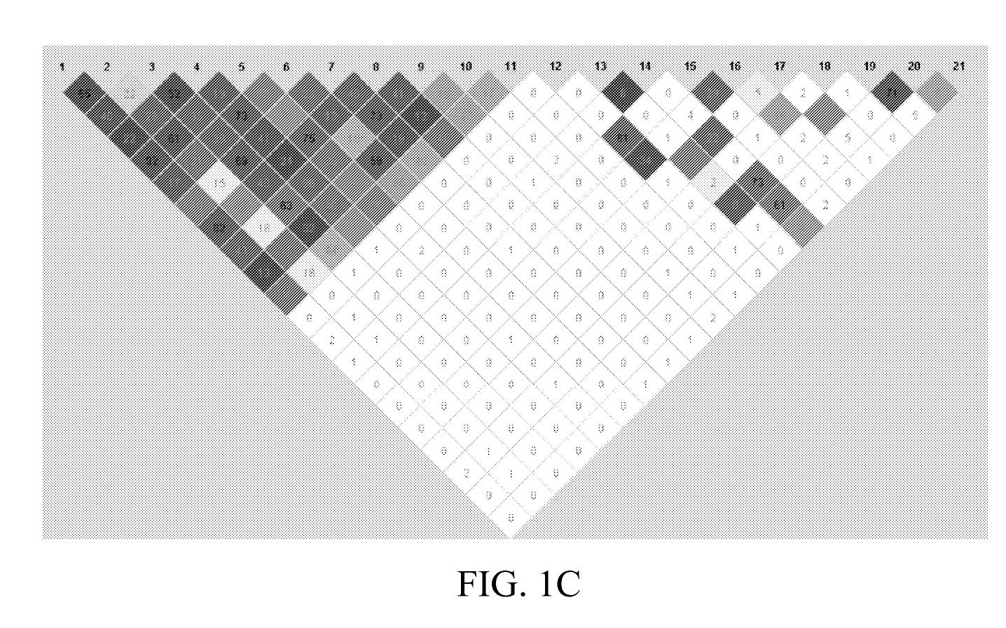

However, rs11022111, located in the promoter of DKK3, showed significant association despite the relatively small sample size (allele χ² p=2.25×10⁻⁵, FIG. 1, Tables 3 & 4).

The association remained significant after a Bonferoni correction for multiple comparisons (P<5.0×10⁻³=0.05/10) and an evaluation for multiple testing (P<5×10⁻⁵ after 10,000 permutations). Further, re-genotyping of 200 samples by a different method (direct sequencing) revealed a sole discrepancy, suggesting a genotyping error rate of 0.5% or less.

To characterize these findings further, second stage genotyping was performed on an additional 534 cases and 493 controls in a Han Chinese cohort from Northern China. Significant association (allele χ² p=1.62×10⁻⁶, Table 3) was again observed. An additional attempt was made to replicate this finding in a third Han Chinese cohort of 632 cases and 808 controls. Again, significant association was found for this cohort (allele χ² p=3.44×10⁻⁸, Table 3). Overall, we observed highly significant association in the Han Chinese cohort (allele χ² p=8.01×10⁻¹⁷, C allele: 16.99% in cases versus 9.71% in controls). The association fits best with a dominant model, (p=5.28×10⁻¹⁷, OR_{dom}=2.04 [1.73, 2.42], CC and CG alleles: 31.8% in cases versus 18.6% in controls, Table 8, FIG. 1).

To investigate whether DKK3 rs11022111 is associated with T2DM in other ethnic populations, a T2DM cohort (913 cases and 337 controls) from a Caucasian population in Utah was genotyped. This cohort was slightly older, and had higher BMI, cholesterol and LDL than the Chinese cohorts, but these differences were not statistically significant (Table 1).

Once again, a significant association with DKK3 rs11022111 was observed (P=1.47×10⁻³ with a dominant model, OR_{dom}=1.56 [1.18, 2.05], CC and CG alleles: 37.2% cases versus 27.6% in controls, Table 8, FIG. 1).

To confirm the findings further, a fourth population was sought to replicate the association. A Japanese cohort was therefore genotyped for the same SNP (2692 cases and 1960 controls). Once again, significant association was observed between this polymorphism and T2DM with similar significance (P=8.9×10⁻⁴ with a dominant model, OR_{dom}=1.23 [1.08, 1.40], CC and CG alleles: 31.76% cases versus 27.2% in controls, Table 8, FIG. 1).

One potential explanation for the robust replication of the association in each cohort is that the interrogated SNP is causally related to T2DM or in complete LD with the true pathogenic allele. Since no information is available from the HapMap data on rs11022111, an additional 39 SNPs were genotyped around the DKK3 locus and adjacent genes in 400 cases and 200 controls from the Utah cohort to survey the haplotype structures within the DKK3 region. It was found that four more SNPs in DKK3 showed significant association with T2DM including rs1552796 (p<0.011), rs6485328 (p<0.049), and rs4307701 (P<0.05) (FIG. 1). In parallel, genotyped several SNPs were also in DKK3 in the combined Han Chinese cohort, where two SNPs were found in LD with SNP rs11022111: rs4307701 (r2=0.20, D′=0.83) and rs11022114 (r2=0.03, D′=0.33); upon testing, the minor alleles of these SNPs were found to also exhibit significant association with T2DM in the Han Chinese cohort (P<0.015 and 0.026 respectively, FIG. 1). Overall, the CG haplotype across rs11022111 and rs4307701 exhibits significant association with T2DM (P=5.28×10⁻⁹) in the Chinese and Utah populations (p<1.2×10⁻³), but the association can be best explained by rs11022111. Furthermore, a re-sequencing effort was done to discover additional variants in DKK3 that contribute to the risk of T2D. One of them, G allele of rs3206824 (coding a missense G335R change), is significantly associated with T2D in the Utah cohort (Table 9).

TCF7L2 has been associated with T2DM in Caucasians (Cauchi et al., 2006; Damcott et al., 2006; Grant et al., 2006; Helgason et al., 2007; Humphries et al., 2006; Munoz et al., 2006; Saxena et al., 2006); its role was therefore assessed in the Han Chinese, Utah and Japanese T2DM cohorts. Significant association was observed between T2DM and the commonly associated TFC7L2 SNP rs7903146 in the Utah cohort (P=2.59×10⁻⁴) and the Japanese cohort (P=3.06×10⁻⁴). A significant association was also observed for the SNP rs7903146 in the Chinese cohort (P=3.33×10-3).

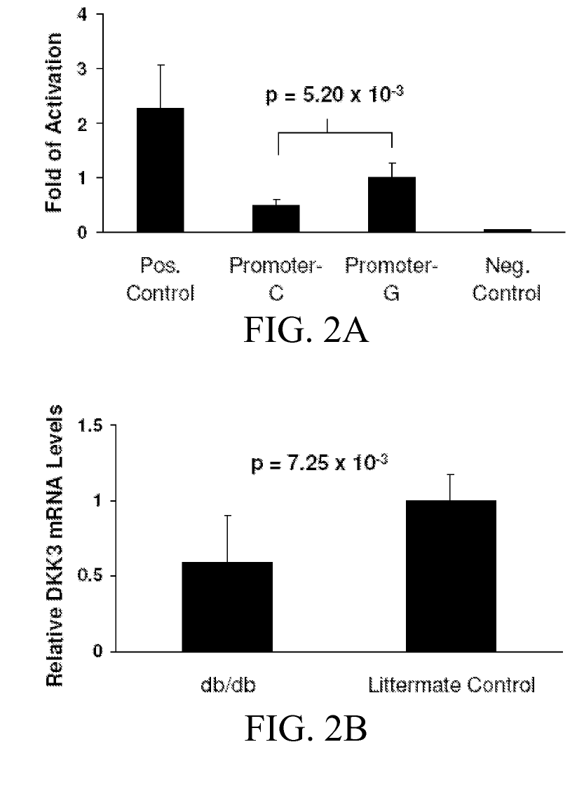

The functional significance of the DKK3 promoter SNP: It was next evaluated whether rs11022111 is causally related to the T2DM and how the minor allele might impact the DKK3 transcript. The SNP lies 965 by upstream of the DKK3 translation start site. Using MatInspector to scan for putative transcription factor binding sites within this region, a highly conserved SP1 enhancer site was identified that is eliminated by the C risk variant (Table 6). To test this computational observation, the DKK3 promoter was cloned with either the C or the G allele and an identical remaining promoter sequence and fused to firefly luciferase cDNA. Transfection of mammalian cells expressing SP1 with either construct confirmed attenuation of expression of the promoter, with the C risk allele resulting in a 2-fold reduced expression of Luciferase as compared to that of the G allele (FIG. 2A, P=0.0052). Consistent with the hypothesis that decreased expression of DKK3 plays a role in the pathogensis of T2DM, it was found that the expression of DKK3 was significantly decreased in the kidney of db/db mice (Sharma, K., et al. 2003) (FIG. 2B, P=0.0073) as compared with littermate controls.

DKK3 is a Wnt agonist: To determine how downregulation of DKK3 might be causally related to T2DM susceptibility, the possible functions of the DKK3 protein were evaluated. The DKK family is composed of four small glycoproteins (DKK1-4), of which DKK3 is the most divergent, and a DKK3-related molecule, DKKL1 (encoded by soggyI; Niehrs, C. 2006). Biochemical and in vivo experiments have demonstrated that DKK1 and DKK2 act primarily as antagonists of β-catenin Wnt signaling by binding (and presumably sequestering) the LRP6 Wnt co-receptors (Mao, B., et al. 2001; Brott, B. K. & Sokol, S. Y. 2002). Some evidence suggests that at least DKK1 might also be involved in β-catenin independent Wnt signaling, including planar cell polarity (PCP) and the Wnt/Ca²⁺ cascade (Niehrs, C. 2006; Lee, A. Y., et al. 2004). The functions of DKK3, however, appear to be different as overexpression of DKK3 in cells or embryos does not suppress Wnt β-catenin signaling (Brott, B. K. & Sokol, S. Y. 2002; Krupnik, V. E., et al. 1999). These findings have led to the suggestion that DKK3 might not be involved in Wnt signaling (Niehrs, C. 2006; Brott, B. K. & Sokol, S. Y. 2002; Krupnik, V. E., et al. 1999). However, DKK3 is also thought to be a tumor suppressor (Hsieh, S. Y., et al. 2004) reportedly acting through modulation of the Wnt/β-catenin signaling pathway (Hoang, B. H., et al. 2004).

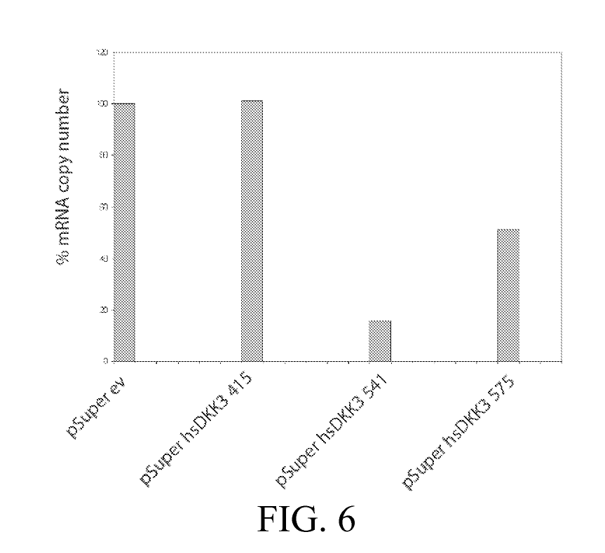

It was sought to either suppress or overexpress DKK3 in mammalian fibroblasts (HEK 293T) and study the effect of these manipulations on Wnt signaling. Thus, three different short hairpins targeting human DKK3 were expressed. Using qRT-PCR, the presence of endogenous DKK3 was confirmed in the cells and tested for the efficacy of RNAi 48 h and 72 h post-transfection. pSuper hsDkk3 541 proved to be the most effective, reducing the amount of DKK3 message by ˜80% after 72 h (FIG. 6).

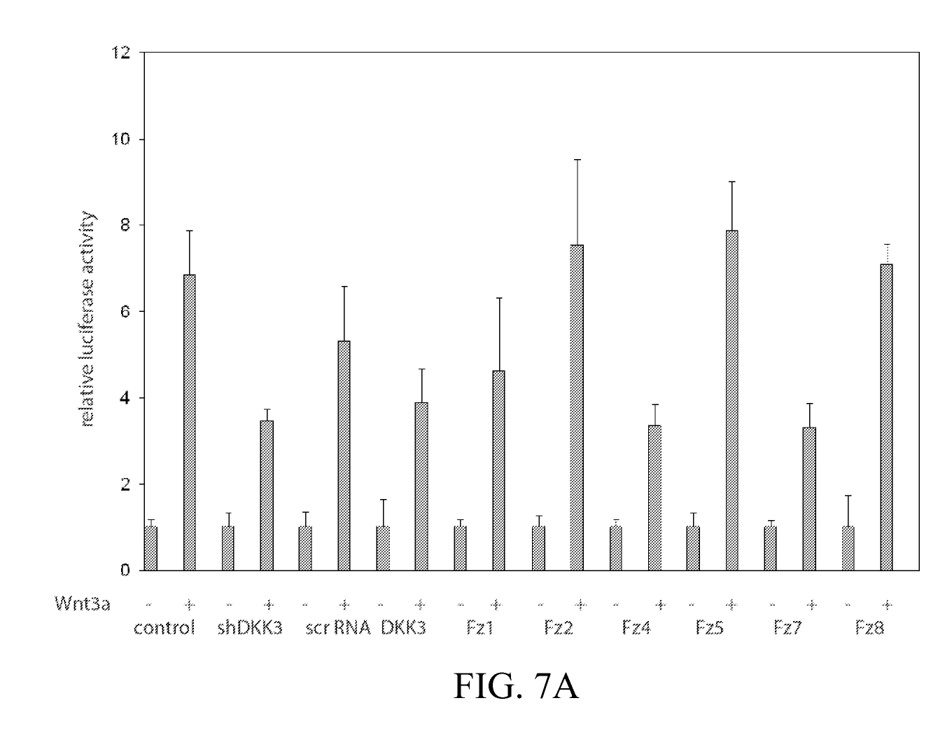

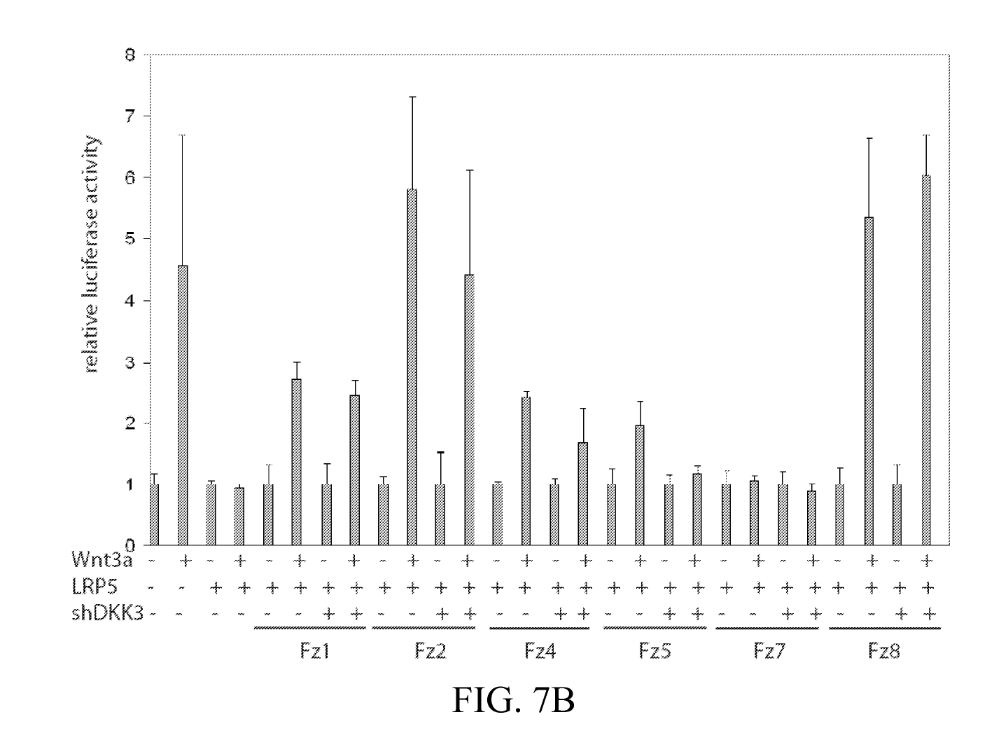

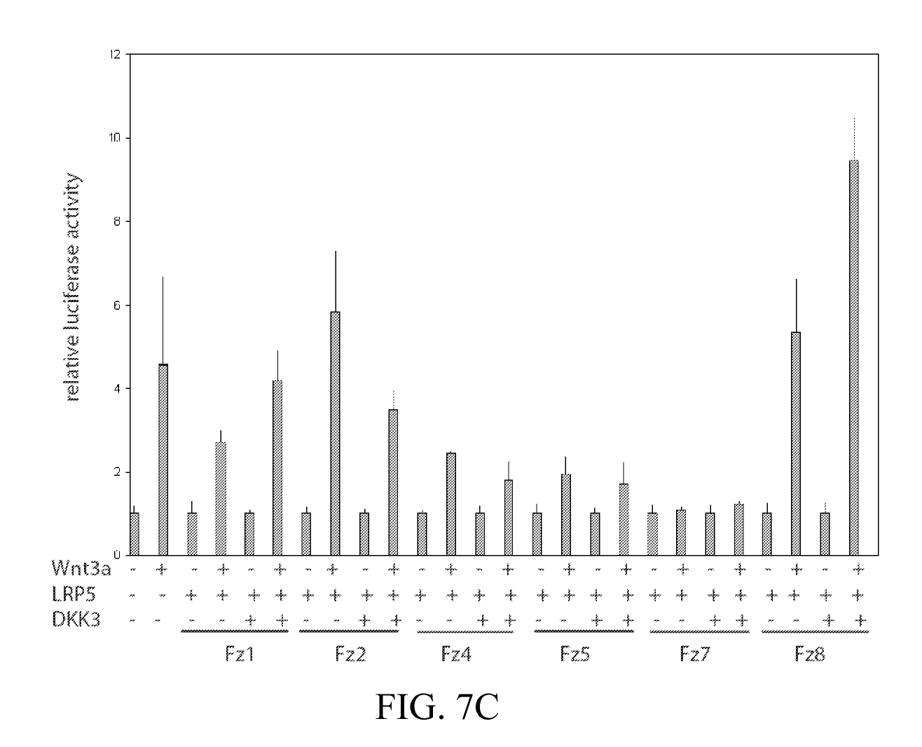

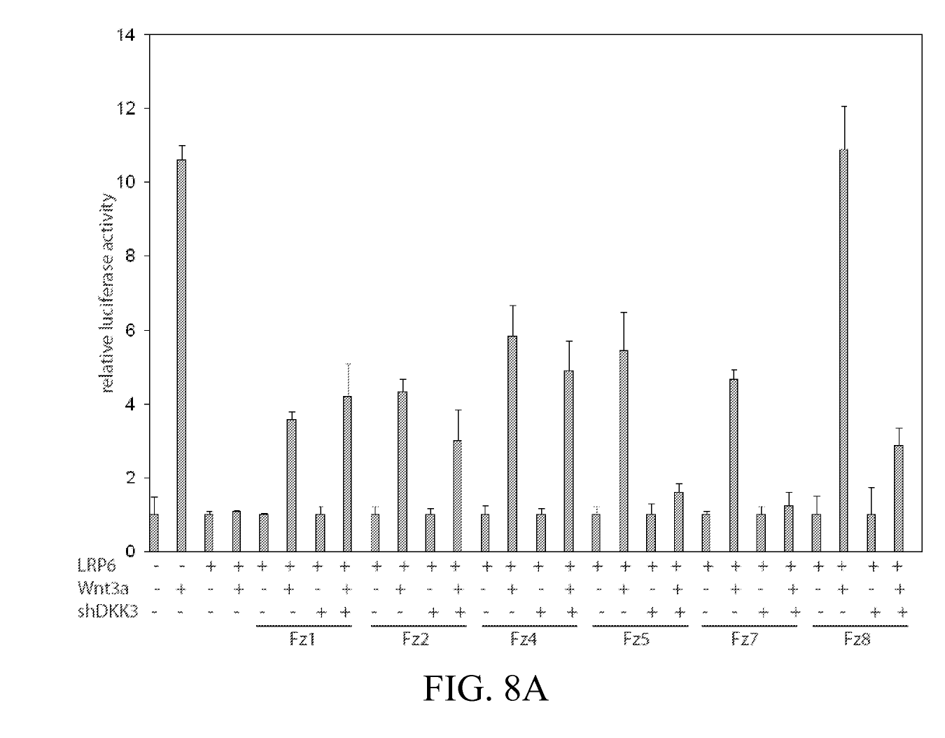

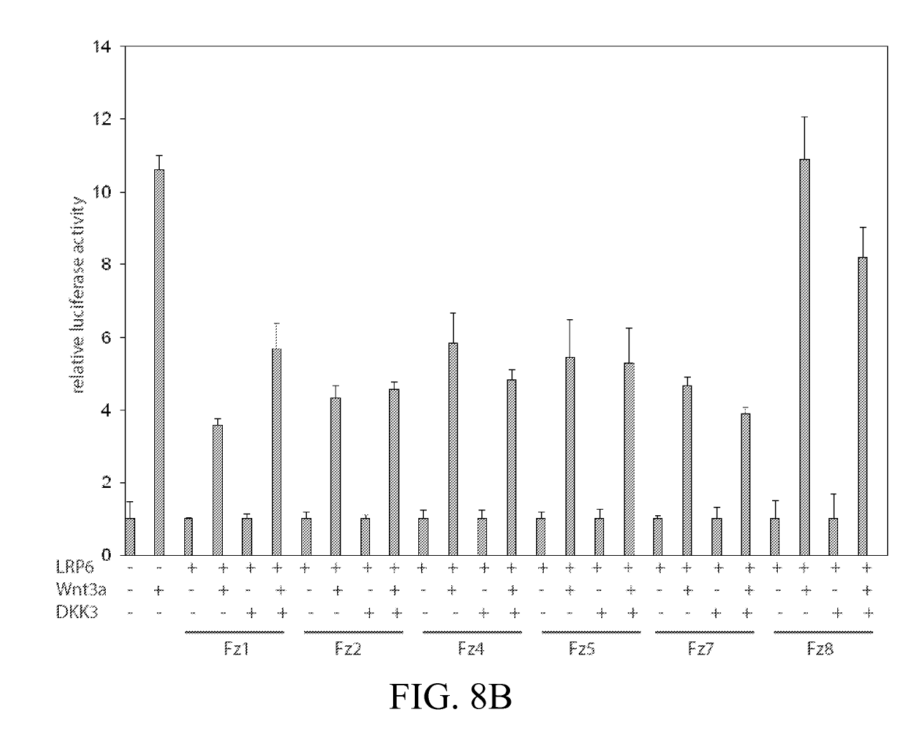

Consistent with previous reports (Mao, B., et al. 2001; Brott, B. K. & Sokol, S. Y. 2002), overexpression of DKK3 did not result in significant changes of β-catenin activity upon Wnt3a stimulation, as assayed by the TOPFlash luciferase reporter (Veeman, M. T., et al. 2003) (FIG. 7A). Likewise, no changes in β-catenin activity were observed upon DKK3 suppression (FIG. 7A). However, because physiologically relevant Wnt activity is typically dependent on the interaction of the ligand with a number of co-receptors, including the Fz family and LRP5/6, it was asked whether DKK3 might influence Wnt activation in a Fz/LRP-dependent manner. Pairwise analyses of DKK3 with each of Fz1-10 or LRP5 and LRP6 showed no changes in β-catenin activation. However, significant effects of DKK3 were observed when each of Fz1-10 was expressed with either LRP5 or LRP6 upon either overexpression or suppression of DKK3 (FIGS. 7 and 8).

Expression of Fz1-10 with LRP5 or LRP6 resulted in increased β-catenin activity in both Wnt stimulated and unstimulated cells (FIGS. 7B and 8B). Interestingly, suppression of endogenous DKK3 in the presence of either LRP5 or LRP6 resulted in attenuation of activity for a subset (but not all) of FZD receptors (Fz-1,2,4,5,7 and 8-FIGS. 7B and 8B), and the effect was TCF/LEF-dependent, since repetition of these experiments with the FOPFlash reporter, which cannot bind β-catenin (Korinek, V., et al. 1997), yielded no activation in this or in any subsequent experiment. Moreover, overexpression of DKK3 augmented the β-catenin activity only in the presence of Fz8 and LRP5, but no other combinations, (FIG. 7B), highlighting the notion that DKK3 is involved in the regulation of β-catenin activity in a specific context, indicating why some previous investigations concluded that DKK3 might not play a role in Wnt signaling (Brott, B. K. & Sokol, S. Y. 2002; Krupnik, V. E., et al. 1999).

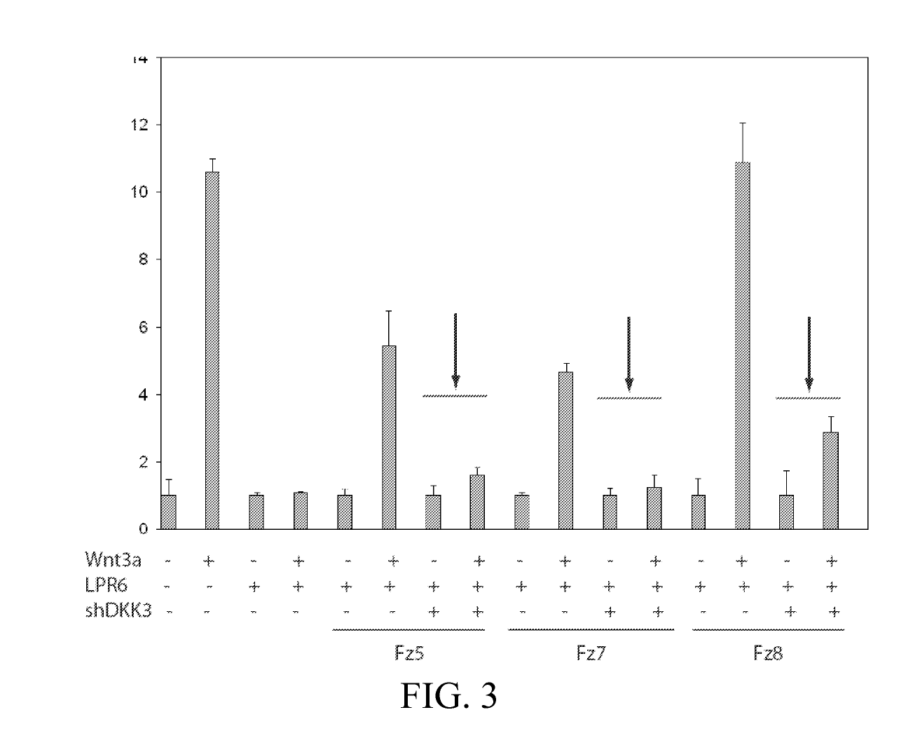

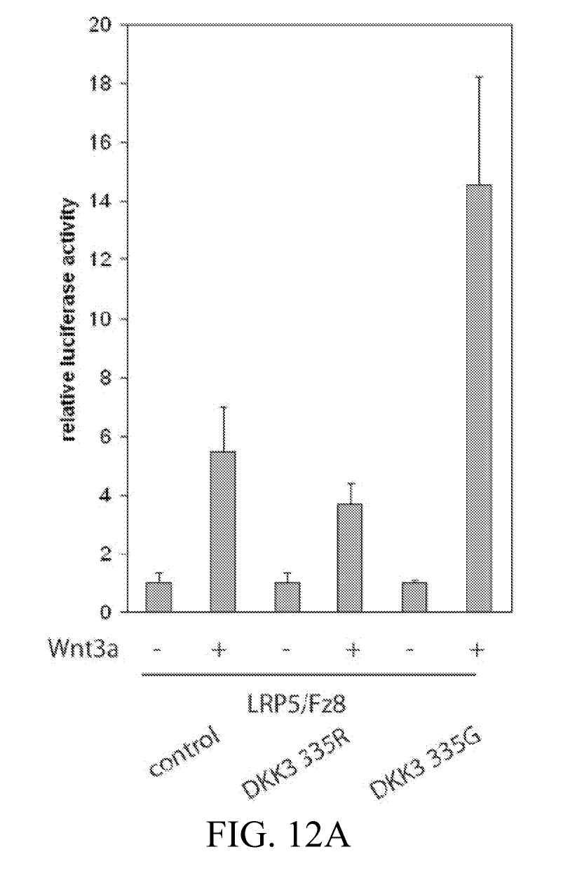

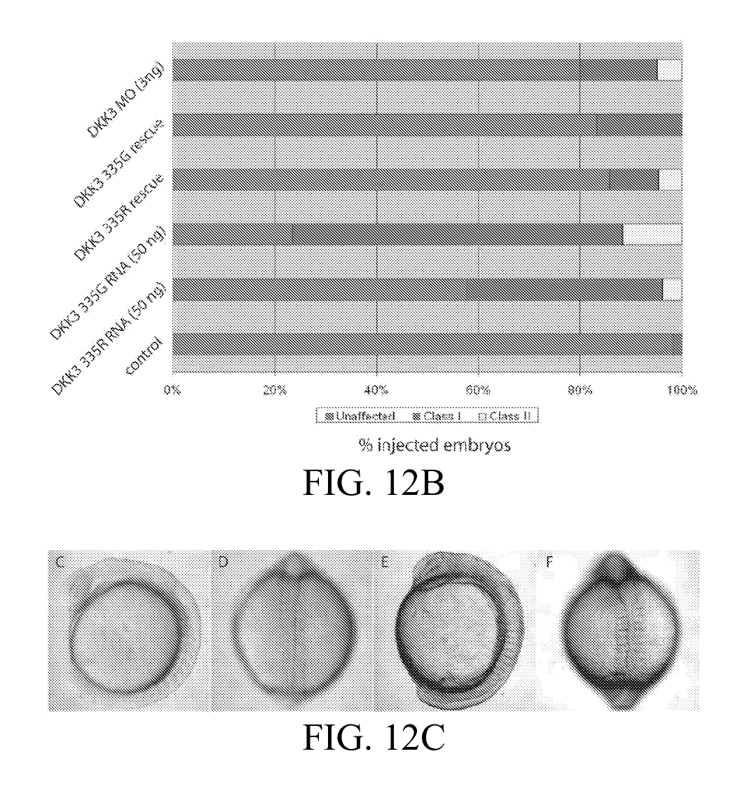

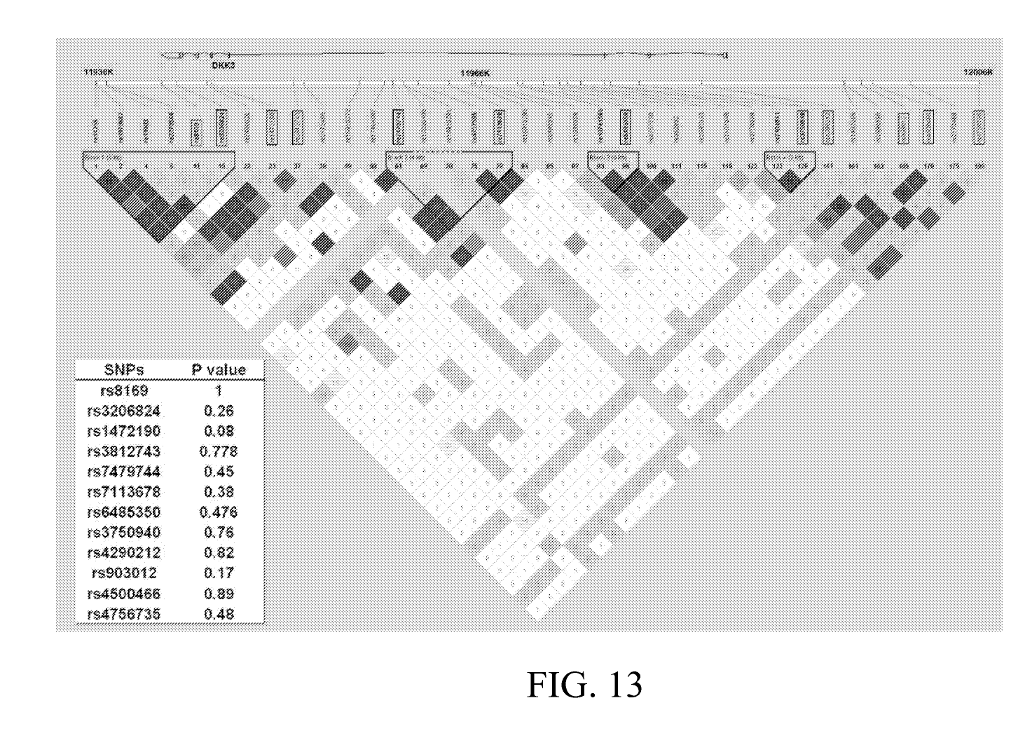

Importantly, the basal levels of β-catenin activity in unstimulated cells were significantly increased, which would mask any potential Wnt-specific effect. Therefore, to assess the role of DKK3 in Wnt-dependent signaling, the relative increase of β-catenin activity in response to Wnt3a was calculated by measuring the fold increase in luciferase activity between unstimulated and Wnt3a stimulated cells (FIGS. 3, 7C and 8A). It was found that neither DKK3 suppression nor overexpression has any significant effect on LRP5-mediated Wnt signaling, irrespective of which Fz co-receptor is used (FIG. 7C). By contrast, DKK3 is necessary for aspects of LRP6 signaling, since suppression of endogenous DKK3 significantly attenuates Wnt activity in a Fz5, Fz7 and Fz8-dependent context (FIG. 3). DKK3 overexpression had no notable effect (FIG. 8B). The functional role of rs3206824 risk allele G coding the 335R was further investigated in the above luciferase expression system and found that 335R is a null allele (FIG. 12A).

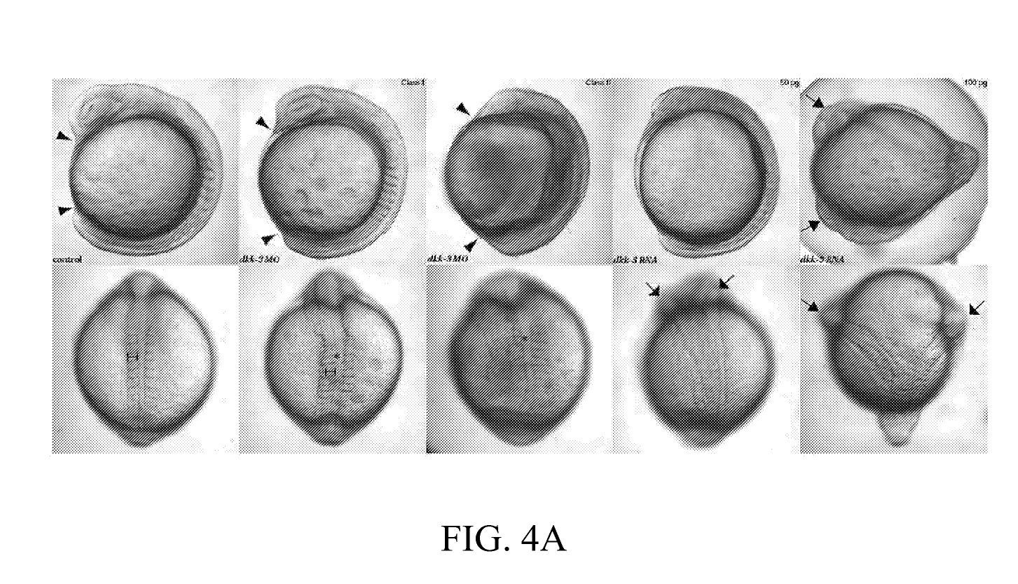

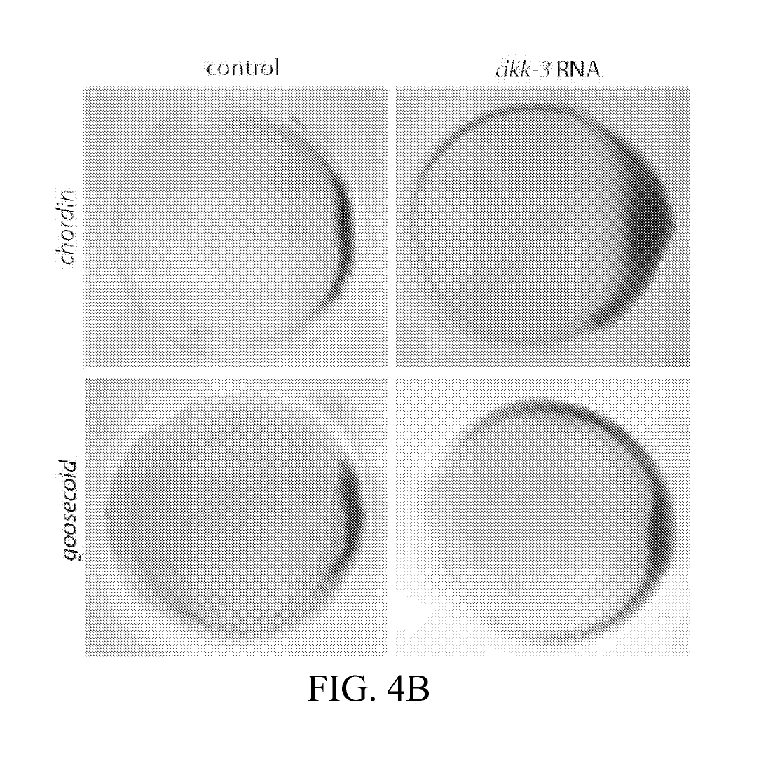

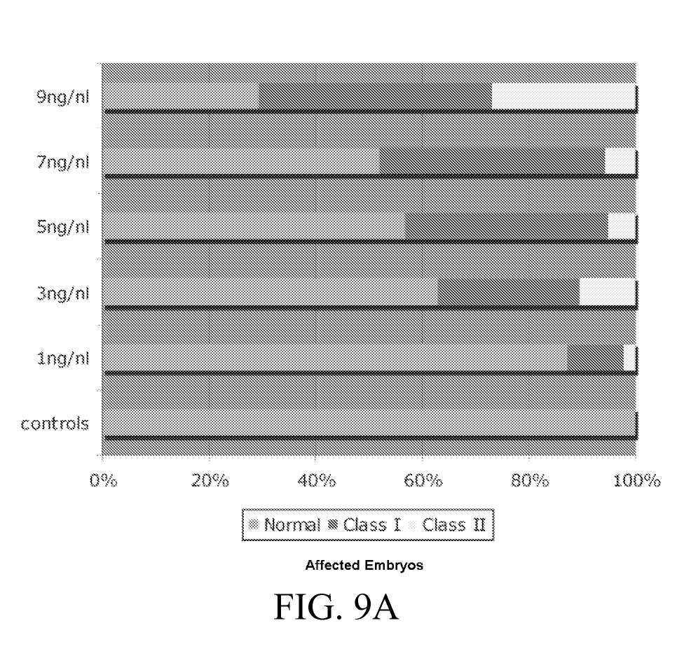

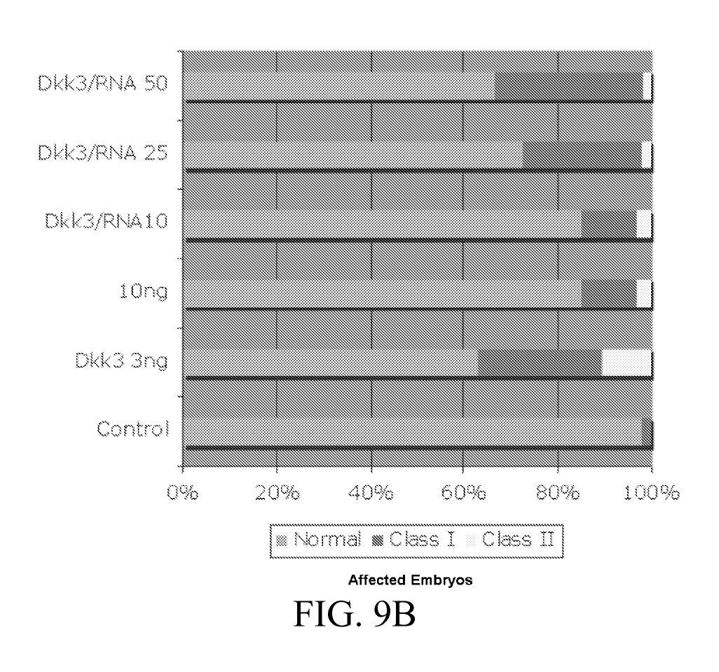

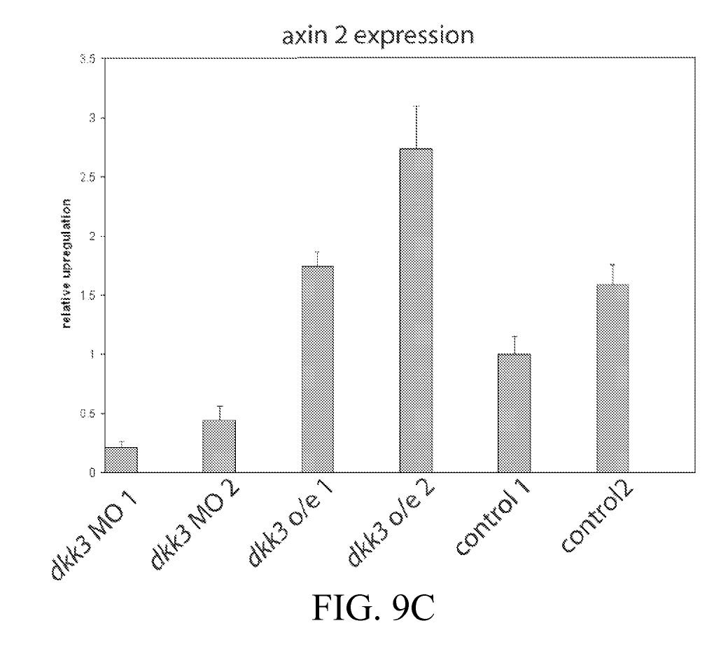

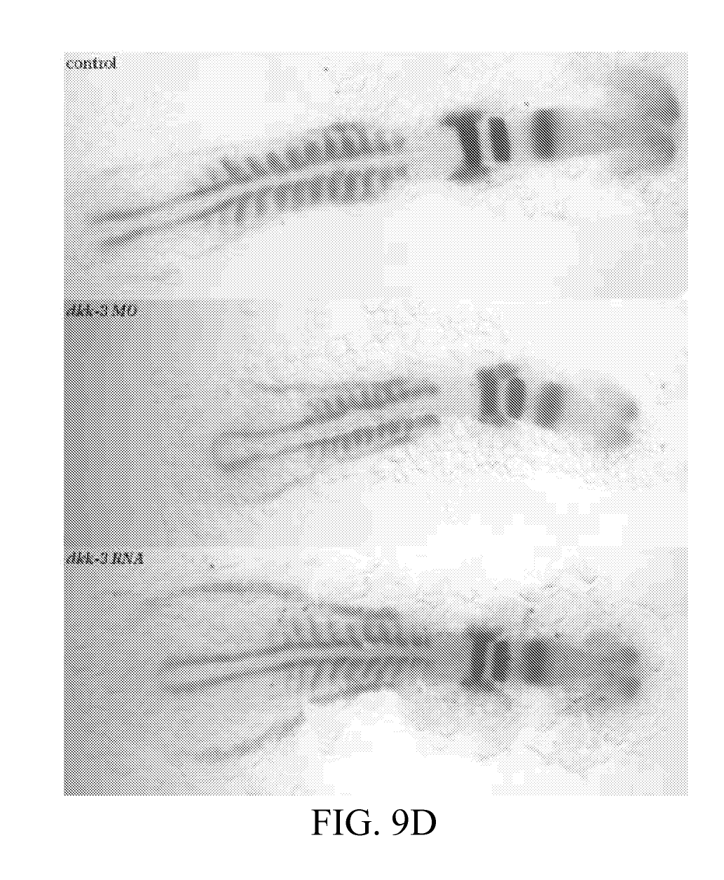

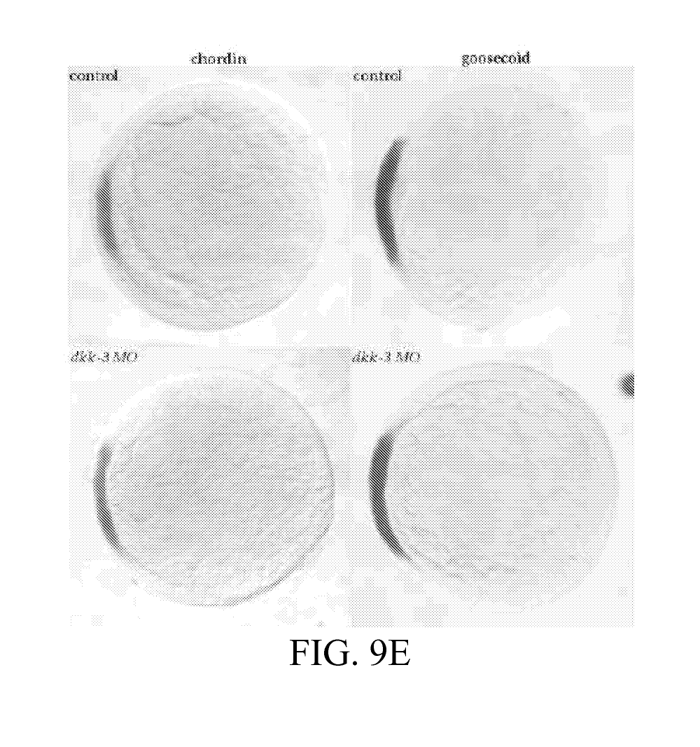

A single copy of a likely DKK3 ortholog was found in the zebrafish genome (65% similarity to human DKK3 but only 37% similar to either DKK1, DKK2 or DKK4), to which a translational blocking morpholino (MO) was designed. Injection of progressively increasing amounts of MO that ranged from 1 ng to 9 ng and masked scoring of embryos (n=100-150 embryos/injection, FIG. 9A) gave rise to characteristic convergence and extension phenotypes that have been described in several Wnt mutants (Heisenberg, C. P., et al. 2000; Kilian, B., et al. 2003). These phenotypes included short body axes, reduced distances between the 3^{rd} rhombomere and the first somite and broad, undulated notochords (FIGS. 4 and 9), as visualized both in live and flatmounted embryos stained with a riboprobe cocktail against MyoD, Pax2 and Krox20 (FIGS. 4 and 9C). Moreover, no expansion of dorsal markers such as goosecoid and chordin was observed, further indicating that DKK3 is not a potent Wnt antagonist (FIG. 9D). The phenotypes were specific, since they could be phenocopied with a second, splice blocking dkk3 MO and were rescued efficiently by a capped dkk3 mRNA that escaped MO suppression (FIG. 9B).

Consistent with the data described above (FIGS. 3, 7 and 8), mild overexpression of dkk3 had modest effects, including dilation of the notochord in ˜50% of our embryos (58/123 embryos, masked scoring), which is consistent with the expansion of dorsal structures seen in various Wnt overexpression embryos (Landesman, Y. & Sokol, S. Y. 1997; McMahon, A. P. 1992), embryos overexpressing β-catenin (Kelly, G. M., et al. 1995), as well as in other dorsalizing manipulations, such as overexpression of fgf8 and the bmp genes (Furthauer, M., et al. 1997; Ramel, M.-C., et al. 2005). More dramatically, dkk3 embryos at the extreme end of phenotypic severity showed complete dorsalization, as judged by the formation of a double axis (FIG. 4, injection of 50 pg and 100 pg dkk3 RNA), a phenotype also seen upon overexpression of fz8 (Itoh, K., et al. 1998), one of the likely DKK3 co-receptors based on the data described above (FIG. 3). In concordance with the observed double axis formation, both chd and gsc show strong expansion of their expression domain (FIG. 4B) providing further evidence of increased β-catenin transcriptional activity and Wnt signaling. the role of rs3206824 risk allele G (335R) was also investigated. Consistent with 335R is a null allele, it was found that 335R produced a markedly wnt deficient phenotype compared to 335G (FIG. 12 B-F).

Finally, to obtain direct evidence for an effect of dkk3 on Wnt β-catenin signaling, expression levels of axin2, an established β-catenin transcriptional target (Jho, E. H., et al. 2002) were assayed for changes. Consistent with the interpretation of the live embryo data, as well as the in vitro luciferase assays, suppression of dkk3 downregulated the transcriptional levels of axin2, while overexpression of dkk3 had the opposite effect (FIG. 9B)

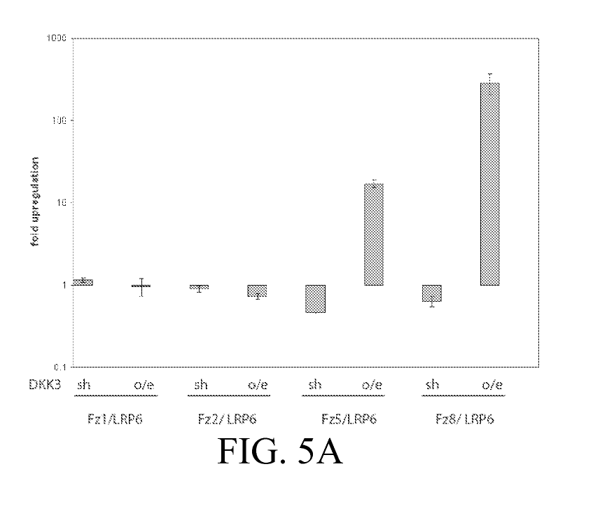

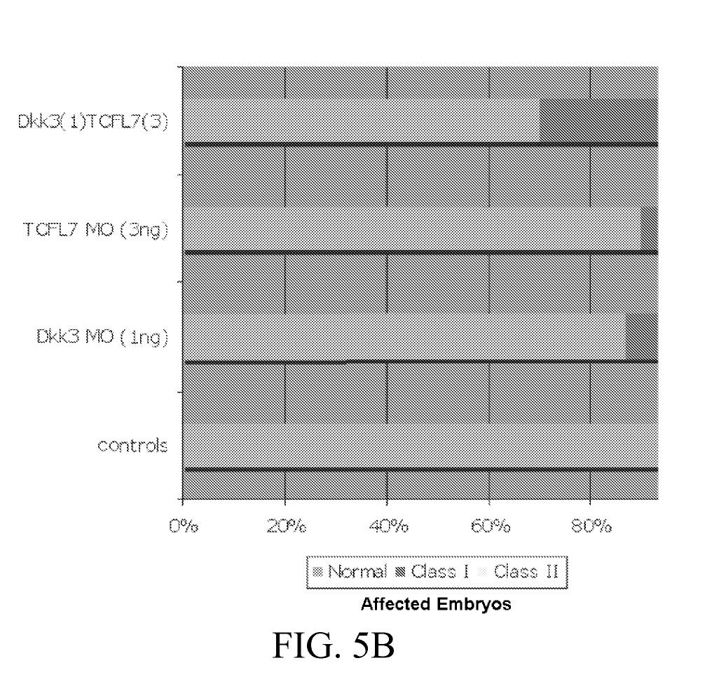

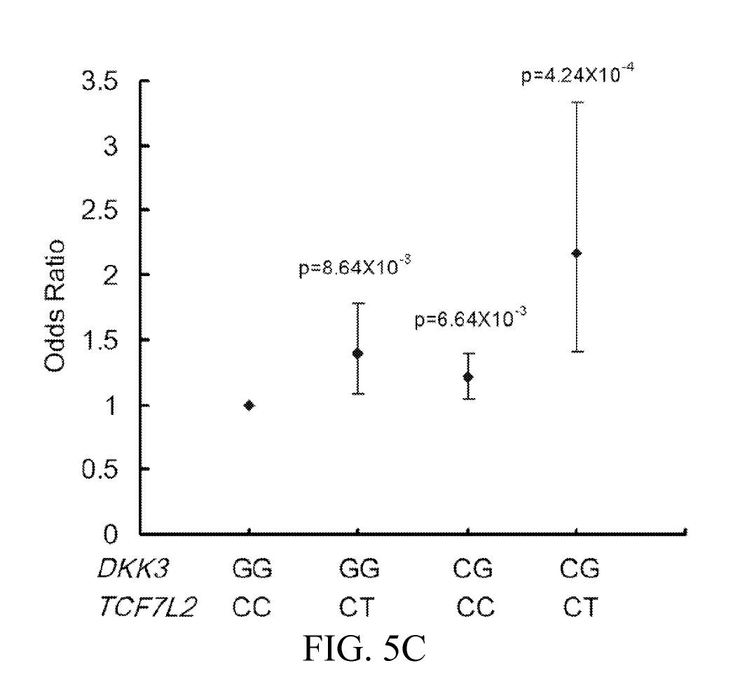

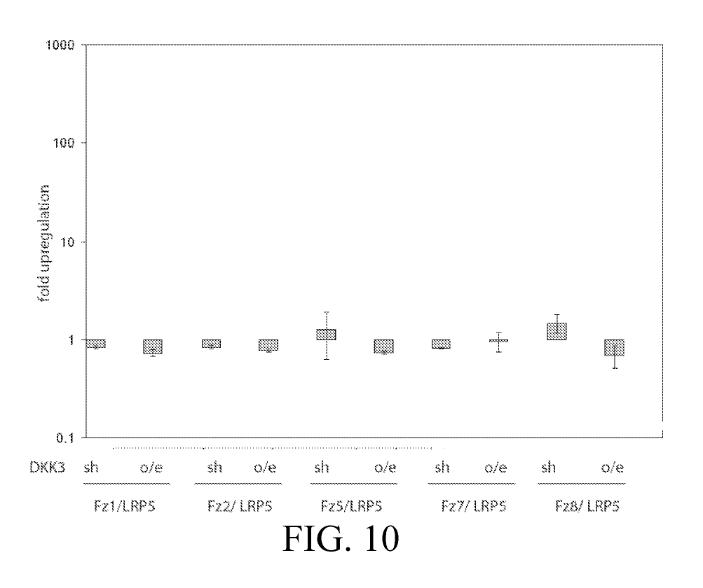

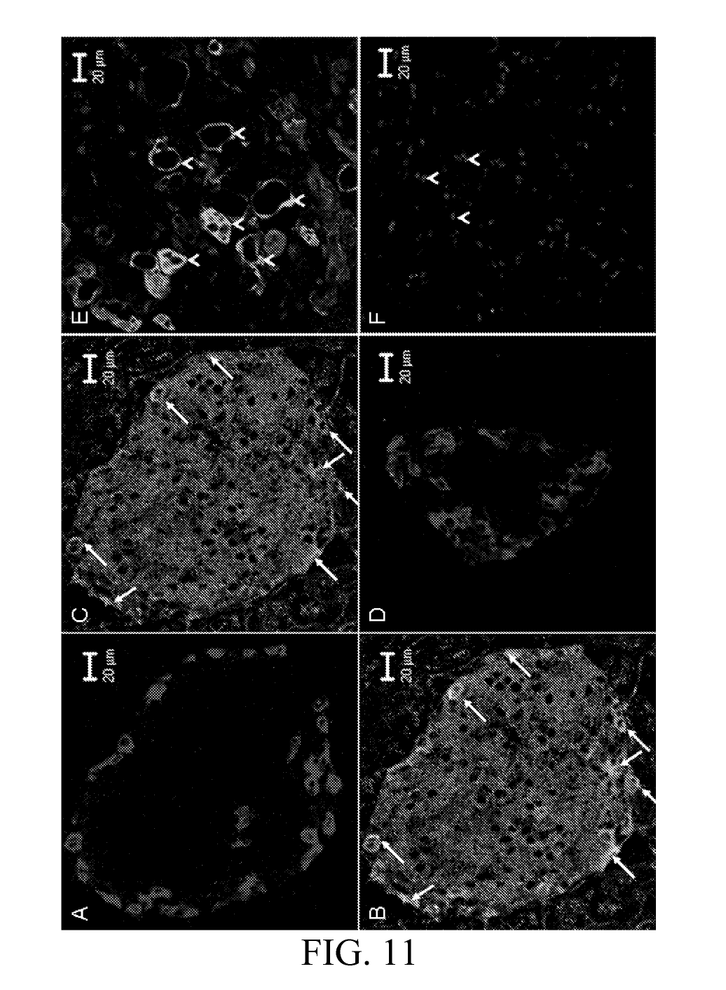

TCF7L2 is regulated by DKK3: Recent reports strongly implicate TCF7L2 as a diabetes susceptibility gene (Grant, S., et al. 2006; Helgason, A., et al. 2007). Decreased expression levels of TCF7L2 in obese diabetic patients (Cauchi, S., et al. 2006) are thought to confer impaired insulin secretion (Munoz, J., et al. 2006; Saxena, R., et al. 2006; Damcott, C., et al. 2006) and possibly insulin resistance (Damcott, C., et al. 2006). Since TCF7L2 is computationally predicted to be a β-catenin target, it was asked whether the observed changes in Wnt signaling upon suppression of DKK3 might impact the levels of endogenous TCF7L2 mRNA. A DKK3 shRNA was therefore co-expressed with LRP6 and Fz5, 7 and 8, since DKK3 appears to act through these co-receptors (FIG. 3), as well as negative controls that included Fz1 and Fz2 and the same Fz series with LRP5. Suppression of DKK3 in the presence of LRP5 or Fz1/Fz2 had no effect on the transcript levels of TCF7L2 (FIGS. 5 and 10). However, in the presence of LRP6, DKK3 modulates TCF7L2 expression in a Fz5, Fz7 and Fz8-dependent manner: suppression of DKK3 attenuated TCF7L2 expression, while overexpression of DKK3 resulted in increased levels of TCF7L2 expression for Fz5 and Fz8 but not Fz7 (>17 fold for DKK3/Fz5/LRP6 and >280 fold for DKK3/Fz8/LRP6; FIG. 5). Since obese diabetic patient were found to have suppressed TCF7L2 expression levels compared to non-diabetic obese patients (Cauchi, S., et al. 2006) these findings suggest that DKK3 is likely upstream of TCF7L2 and that downregulation of either transcript can predispose a patient to T2DM through this pathway.

These observations were corroborated further in our zebrafish model. Consistent with the notion that both DKK3 and TCF7L2 are components of the Wnt signaling pathway, suppression of tcf712 with a translation blocking MO phenocopied dkk3 morphants. More pertinently to the transcriptional DKK3/TCF7L2 data, while single suppression of either tcf712 or dkk3 at subeffective morpholino concentrations show only mild effects in a minority of embryos (˜10-15%, n>100 embryos scored blind), concurrent subeffective suppression of both transcripts yielded likely Wnt defects in some 30% of embryos (FIG. 5B), indicating that the effect of susceptibility alleles in two genes on the phenotype are additive. Finally, it was reasoned that the genetic interaction in zebrafish embryos can inform the human genetic studies. Given the dominant model for each of TCF7L2 and DKK3, a synergistic effect of these two genes in humans could manifest primarily as an excess of double heterozygotes for both loci in T2DM patients compared to controls. To test this possibility, two-locus analysis was performed in each of the three cohorts; in each case, two-locus odds ratios indicate that DKK3 and TCF7L2 act additively to increase susceptibility to T2DM and fits best with a dominant model (FIG. 5C).

iii. Discussion

Disclosed herein is the identification of DKK3 as a new susceptibility locus for T2DM. Based on the initial hypothesis that Wnt pathway misregulation might be involved in mediating features of the T2DM clinical presentation, it is disclosed that variations in this gene contribute a much as 16.7% of the genetic load of T2DM in Chinese populations while TCF7L2 does not contribute significantly to T2DM in the same cohort.

Of interest, the disease-associated promoter SNP rs11022111 is neither present in any commercially available SNP arrays, nor in LD with reported HapMap SNPs, which may explain why DKK3 has not been detected in previous T2DM genome-wide association studies.

The disclosed data also indicate that Wnt signaling is involved in the etiology of T2DM. The in vitro results indicate that DKK3, in contrast to the other three known members of the DKK family, is a Wnt agonist and not an inhibitor of β-catenin signaling. Furthermore, in HEK293T cells, DKK3 affects Wnt activity specifically through a subset of Wnt co-receptors, most prominently Fz5, Fz7, and Fz8, specifically mediated by LRP6 but not LRP5. It is notable that an LRP6 mutation that attenuates β-catenin transcriptional response was associated recently with metabolic syndrome (Mani, A., et al. 2007).

Dkk3 is expressed in mouse pancreas and in mouse preadipocytes and adipocytes (FIG. 10). Previous studies have shown the involvement of both canonical (Wells, J., et al. 2007) and non-canonical Wnt signaling (Kim, H., et al. 2005) in pancreatic development, with both signals transmitted through a specific combination of Fz ligands and co-receptors such as Wnt5a/Fz2 (Kim, H., et al. 2005) for differentiation of pancreatic islet cells. Thus, as disclosed herein, moderate suppression of DKK3 in either of these tissues can lead to modest loss of β-catenin signaling (and also modest loss of non-canonical Wnt signaling) that can affect the proliferation and/or differentiation of islet β-cells, or the ability to respond to glucose. In the adult pancreas, LRP5 is important for insulin secretion in response to a rise in glucose levels (Fujino, T., et al. 2003). Pancreatic islets of LRP5 deficient mice have a significant reduction in ATP and Ca²⁺ levels in response to a glucose stimulus as well as decreased expression levels of genes involved in glucose sensing Fujino, T., et al. 2003).

Likewise, Wnt signaling plays a pivotal role in adipogenesis and adipogenic differentiation, which, in light of the disclosed data, can provide some clues as to the link between obesity and the development of T2DM. Overexpression of a dominant negative form of TCF₄ (TCF7L2 ortholog) or Axin, which targets β-catenin for degradation, causes preadipocytes to differentiate (Ross, S., et al. 2000), while adult myoblasts convert into adipocytes upon loss of canonical Wnt signaling (Ross, S., et al. 2000). The Wnt signal is mediated through Fz1, 2, and possibly 5 as well as both LRP5 and LRP6 (Bennett, C., et al. 2002). In addition, mice that express Wnt10b under the FABP4 promoter are resistant to diet-induced obesity (Bennett, C., et al. 2002). In these mice, Wnt10b seems to inhibit the differentiation of preadipocytes into white and brown adipose tissue (Bennett, C., et al. 2002).

### I. Tables

TABLE 1
Characteristics of T2DM Cases and Matched Controls in Chinese and Utah cohorts

| | | | | Average | | |
|---|---|---|---|---|---|---|
| | Total | # Male | HbA1c | Age | FPG | BMI |
| | Number | (%) | (%) | (years) | (mmol/L) | (kg/m2) |
| Southern Chinese | 338 | 160 | 8.60 | 60.3 | 8.22 | 24.88 |
| T2DM cohort | | (47.3%) | 6.15-16.9) | (43-82) | (4.18-17.13) | (17.36-40.06) |
| Southern Chinese | 223 | 107 | 4.72 | 60.9 | 4.79 | 24.03 |
| Control cohort | | (48.0%) | (3.01-5.99) | (45-81) | (3.63-5.71) | (14.15-40.93) |
| Northern Chinese | 534 | 293 | 8.20 | 59.7 | 7.78 | 24.46 |
| T2DM cohort | | (54.9%) | (5.58-15.60) | (45-79) | (4.23-18.20) | (16.64-32.83) |
| Northern Chinese | 493 | 271 | 4.59 | 62.5 | 4.60 | 23.69 |
| Control cohort | | (55.0%) | (2.64-5.71) | (49-78) | (3.44-5.59) | (14.51-35.55) |
| Third Chinese | 635 | 324 | 8.40 | 58.5 | 8.44 | 24.92 |
| Replication | | (51.0%) | (6.21-16.2) | (46-89) | (4.27-18.01) | (16.56-41.86) |
| T2DM cohort | | | | | | |
| Third Chinese | 808 | 402 | 4.64 | 61.9 | 4.82 | 23.76 |
| Replication | | (49.8%) | (2.70-5.82) | (47-87) | (3.57-5.69) | (13.77-42.1) |
| Control cohort | | | | | | |
| Utah Caucasian | 913 | 528 | 8.12 | 68.9 | 9.58 | 30.95 |
| T2DM cohort | | (57.8%) | (5.00-13.00) | (33-94) | (3.30-21.00) | (17.10-70.40) |
| Utah Caucasian | 337 | 205 | 4.75 | 75.3 | 5.20 | 26.40 |
| Control cohort | | (60.8%) | (3.20-5.95) | (60-95) | (3.51-5.65) | (15.40-37.30) |
| Japanese T2DM | 2692 | 1698 | 7.40 | 62.8 | 9.00 | 23.90 |
| cohort | | (63.1%) | (5.0-17.50) | (40-97) | (2.30-43.20) | (14.40-50.80) |
| Japanese Control | 1960 | 1062 | 4.70 | 57.6 | 5.10 | 23.20 |
| cohort | | (54.2%) | (3.40-6.20) | (41-77) | (2.00-6.60) | (14.60-35.70) |

| | Systolic | Diastolic | | | |
|---|---|---|---|---|---|
| | BP | BP | Cholesterol | Triglycerides | LDL |
| | (mmHg) | (mmHg) | (mmol/L) | (mmol/L) | (mmol/L) |
| Southern Chinese | 139.09 | 78.78 | 5.75 | 2.09 | 3.76 |
| T2DM cohort | (105-185) | (55-110) | (3.44-9.30) | (0.42-9.06) | (1.98-5.81) |
| Southern Chinese | 119.76 | 75.88 | 4.97 | 1.25 | 3.01 |
| Control cohort | (90-150) | (55-100) | (2.82-7.65) | (0.48-3.65) | (1.07-6.65) |
| Northern Chinese | 138.21 | 78.11 | 5.58 | 1.91 | 3.62 |
| T2DM cohort | (100-180) | (55-120) | (3.59-8.71) | (0.49-7.62) | (1.99-6.69) |
| Northern Chinese | 129.40 | 77.51 | 5.08 | 1.49 | 3.37 |
| Control cohort | (90-148) | (54-98) | (2.82-7.28) | (0.43-5.15) | (1.12-6.36) |
| Third Chinese | 141.8 | 79.81 | 5.82 | 2.01 | 3.91 |
| Replication | (101-176) | (55-124) | (3.20-8.94) | (0.43-8.91) | (1.85-5.93) |
| T2DM cohort | | | | | |
| Third Chinese | 118.5 | 76.08 | 4.87 | 1.42 | 2.97 |
| Replication | (94-145) | (62-94) | (2.84-7.75) | (0.46-3.61) | (1.11-6.85) |
| Control cohort | | | | | |
| Utah Caucasian | Not | Not | 6.17 | 1.78 | 4.09 |
| T2DM cohort | available | available | (2.43-9.70) | (0.45-6.98) | (1.88-8.32) |
| Utah Caucasian | Not | Not | 5.71 | 1.90 | 3.5 |
| Control cohort | available | available | (2.87-7.62) | (0.43-3.67) | (1.07-6.73) |
| Japanese T2DM | 134.10 | 76.80 | 5.43 | 1.41 | 3.25 |
| cohort | (84-209) | (46-120) | (2.50-12.10) | (0.35-11.70) | (0.90-6.85) |
| Japanese Control | 125.90 | 76.40 | 4.84 | 1.52 | 3.14 |
| cohort | (66-195) | (43-116) | (2.00-8.80) | (0.32-8.90) | (0.85-6.10) |

TABLE 2
List of primers used in SNP genotyping for candidate genes involved
in the Wnt pathway

| | | | | | |
|---|---|---|---|---|---|
| | | | | Taqman® | Restr. |
| SNP | Forward primer | Reverse Primer | Snapshot Primer | Assay ID | Enzyme |

SNPs in the DKK3 Region
Gene = DKK3

| | | | | | |
|---|---|---|---|---|---|
| rs1043179 | atatgcgactgcgaacactg | gcagttgaagtgatttatgcttg | aggtgtcatggactgttgcc | | |
| | (SEQ ID NO: 12) | (SEQ ID NO: 13) | (SEQ ID NO: 14) | | |
| rs10741562 | agcgcaatacatgcaaggtt | tgtctacctcaactgtggcttc | tatcatgatcattgaaca | | |
| | (SEQ ID NO: 15) | (SEQ ID NO: 16) | (SEQ ID NO: 17) | | |
| rs10765915 | | | | C_1205905_10 | |
| rs10831686 | tcttcacccagtctacacta | atcgccagggattaaagagg | atacactatcagaaccaaga | | |
| | atgg | (SEQ ID NO: 19) | tatt | | |
| | (SEQ ID NO: 18) | | (SEQ ID NO: 20) | | |
| rs10831693 | ttggggcatacagagtttcc | aaggaaaagcctgtcccagt | catataagttaggtgaagac | | |
| | (SEQ ID NO: 21) | (SEQ ID NO: 22) | ttgactctta | | |
| | | | (SEQ ID NO: 23) | | |
| rs10831711 | ggtaagacaggctgcagagg | gagttgccgataggtcttgc | | | BsaHI |
| | (SEQ ID NO: 24) | (SEQ ID NO: 25) | | | |
| rs11022079 | ggaaatggtgaatgctgttg | aagaggattaggcttcaaat | tggggaagacagtgttcatga | | |
| | (SEQ ID NO: 26) | cg | caggtttataggtccgcttcc | | |
| | | (SEQ ID NO: 27) | aagagaaggttctg | | |
| | | | (SEQ ID NO: 28) | | |
| rs11022098 | gcagggaaattttgaaacca | gcaaagttccatgccaaaat | | | ApaLI |
| | (SEQ ID NO: 29) | (SEQ ID NO: 30) | | | |
| rs11022108 | cctctgcttccaggttcaag | atcctttggcattgctcatc | caaagtgctgggattacaggc | | |
| | (SEQ ID NO: 31) | (SEQ ID NO: 32) | ctgagccatcacgcctggc | | |
| | | | (SEQ ID NO: 33) | | |
| rs11022111 | ccctcctcttctgcctcag | agactaaggccagcggtgat | agccatctccttcactgcctg | | |
| | (SEQ ID NO: 34) | (SEQ ID NO: 35) | cccctgc | | |
| | | | (SEQ ID NO: 36) | | |
| rs11022112 | ccctcctcttctgcctcag | agctgcaagtgcgttatcct | gcctccaggcttgcaacagcc | | |
| | (SEQ ID NO: 37) | (SEQ ID NO: 38) | caccgaggttggggg | | |
| | | | (SEQ ID NO: 39) | | |
| rs11022114 | gggaaaagacaccaggat | ctcacccacccacagaaagt | agccactaatgtaatgga | | |
| | ca | (SEQ ID NO: 41) | (SEQ ID NO: 42) | | |
| | (SEQ ID NO: 40) | | | | |
| rs11022119 | accccaatgtttccactt | tgggctacagggtggtatgt | ccctggtcccaccagcctgac | | |
| | ga | (SEQ ID NO: 44) | tccttttccccaattagcact | | |
| | (SEQ ID NO: 43) | | (SEQ ID NO: 45) | | |
| rs11022134 | tcctcccagctttattga | atggggagatgttggtgaaa | Gtagttaccatttttttgtgt | | |
| | gg | (SEQ ID NO: 47) | gtg | | |
| | (SEQ ID NO: 46) | | (SEQ ID NO: 48) | | |
| rs11544814 | gtaggggagctgcgttttc | tccagactttttggcaagga | Tagctgagagccgggccgggc | | |
| | (SEQ ID NO: 49) | (SEQ ID NO: 50) | ttgact | | |
| | | | (SEQ ID NO: 51) | | |
| rs11544815 | gtaggggagctgcgttttc | cgtgtcctccatcagttcct | Ttggggccaccctgctgtgc | | |
| | (SEQ ID NO: 52) | (SEQ ID NO: 53) | (SEQ ID NO: 54) | | |
| rs11606956 | cggagatcacaggttgaa | gacgttcttttcccctctcc | tgatggagaaggaagttccct | | |
| | gc | (SEQ ID NO: 56) | ggaagagggcaggggagcgcc | | |
| | (SEQ ID NO: 55) | | tctcca | | |
| | | | (SEQ ID NO: 57) | | |
| rs12278824 | tgccacctgtgtctgtatgc | ggaaaggactttgcagtgga | ggagtgtagtggcatgatctc | | |
| | (SEQ ID NO: 58) | (SEQ ID NO: 59) | ggttcactgcagccttgatct | | |
| | | | ctggggctcatt | | |
| | | | (SEQ ID NO: 60) | | |
| rs12576599 | atatcgaactgccccaagtg | cagatctggtttgggctcat | aaactacaaacgaaaaaaaga | | |
| | (SEQ ID NO: 61) | (SEQ ID NO: 62) | aaggaaagaaaagaa | | |
| | | | (SEQ ID NO: 63) | | |
| rs12791723 | tttccaagccaacctctgtc | gggacgcatacaaatctggt | caggagctgcaagtgcgtta | | |
| | (SEQ ID NO: 64) | (SEQ ID NO: 65) | (SEQ ID NO: 66) | | |
| rs1472190 | gccactcacctctctggaag | ggaaaaagggaacagcacaa | gatccatgagtcctcccggtg | | |
| | (SEQ ID NO: 67) | (SEQ ID NO: 68) | ccatttttccagc | | |
| | | | (SEQ ID NO: 69) | | |
| rs1493208 | gaagagcttgccaacaaagg | tgtttctcttcccctcatgg | tcactaaccagagtggtaact | | |
| | (SEQ ID NO: 70) | (SEQ ID NO: 71) | gagggcaagtctcttgctttt | | |
| | | | aag | | |
| | | | (SEQ ID NO: 72) | | |
| rs1552796 | gggactccatcctgaagtga | tcctcgggacaacaaagaag | gaatctgcccagaggtcaccc | | |
| | (SEQ ID NO: 73) | (SEQ ID NO: 74) | agcttgactggactggagc | | |
| | | | (SEQ ID NO: 75) | | |
| rs2291598 | cctgagagtgagggttaggg | tgggaggttatgctccattt | aggtccagaagccggctggcg | | |
| | (SEQ ID NO: 76) | (SEQ ID NO: 77) | gggtcatggcaaagctcgccc | | |
| | | | tcca | | |
| | | | (SEQ ID NO: 78) | | |
| rs2403567 | ggccatactacccaaagcaa | tgggctctttattttgttc | caagagtctgaatagccaaca | | |
| | (SEQ ID NO: 79) | ca | caattttaagcaaaaagaata | | |
| | | (SEQ ID NO: 80) | aagctg | | |
| | | | (SEQ ID NO: 81) | | |
| rs3206824 | gaccaagatggggagatcct | taggaagaagcctggtca | aggacctggagaggagcctga | | |
| | (SEQ ID NO: 82) | gc | ctgaagagatggcgctg | | |
| | | (SEQ ID NO: 83) | (SEQ ID NO: 84) | | |
| rs3750940 | gatgagcaatgccaaaggat | cattcatttgctggttgtgc | agaagtgccacacaaaagct | | |
| | (SEQ ID NO: 85) | (SEQ ID NO: 86) | (SEQ ID NO: 87) | | |
| rs3812743 | ccgggaacacagcatagatt | cttgtcctctcctcctgcac | tcagagtctttgattttccag | | |
| | (SEQ ID NO: 88) | (SEQ ID NO: 89) | tttagag | | |
| | | | (SEQ ID NO: 90) | | |
| rs3896391 | cctggcgaagttaccagatt | cacccccagaaagctaatga | actgcctttctctgcagcact | | |
| | (SEQ ID NO: 91) | (SEQ ID NO: 92) | ccccaca | | |
| | | | (SEQ ID NO: 93) | | |
| rs4288751 | agcaagtccagcctgaaaa | cccaccaatgctaggaggta | aattctgcttctttattataa | | |
| | (SEQ ID NO: 94) | (SEQ ID NO: 95) | gttatttgttatttc | | |
| | | | (SEQ ID NO: 96) | | |
| rs4307701 | agggatctggcatctcattg | tgtttgactggggtgtctga | atcaatttctatgtttattca | | |
| | (SEQ ID NO: 97) | (SEQ ID NO: 98) | aaagaaact | | |
| | | | (SEQ ID NO: 99) | | |
| rs4757519 | | | | C_27912496_10 | |
| rs6485328 | gagtcagcagtgtgggtgaa | ggttccattgcaactgtcct | tgtagggctgagacaggcttt | | |
| | (SEQ ID NO: 100) | (SEQ ID NO: 101) | tgcgcccacacct | | |
| | | | (SEQ ID NO: 102) | | |
| rs6485350 | ggactgagaagtgcctttgc | ctgatgggctgagtcacaga | aaaacgtagcaggtactcaa | | |
| | (SEQ ID NO: 103) | (SEQ ID NO: 104) | (SEQ ID NO: 105) | | |
| rs7113678 | ttctttctggggaaggaggt | gatggggtgtcagacttgct | | | NcoI |
| | (SEQ ID NO: 106) | (SEQ ID NO: 107) | | | |
| rs7478946 | ccacctactccagcaaccat | gtgctctgggtggagaagag | | | AseI |
| | (SEQ ID NO: 108) | (SEQ ID NO: 109) | | | |
| rs7479744 | ctcttctccacccagagcac | ctggagagcaaccaagaagg | cacagccgtctagagaaaaaa | | |
| | (SEQ ID NO: 110) | (SEQ ID NO: 111) | ggcctgt | | |
| | | | (SEQ ID NO: 112) | | |
| rs7480815 | gggactccatcctgagtga | tcctcgggacaacaaagaag | gtgacctctgggcagattcta | | |
| | (SEQ ID NO: 113) | (SEQ ID NO: 114) | tccttaagtctcagtttccac | | |
| | | | atct | | |
| | | | (SEQ ID NO: 115) | | |
| rs7936742 | tgatccagtcaccatccaaa | ctgggaaaacaatccagcac | cataggcatttagaccagggc | | |
| | (SEQ ID NO: 116) | (SEQ ID NO: 117) | acccagtgacacatcttttct | | |
| | | | (SEQ ID NO: 118) | | |
| rs8169 | gatgaatacatggtggcaac | atgactgagcgtagcataca | tggcgtagagttcagtgttcg | | |
| | ag | gg | cagtcgcata | | |
| | (SEQ ID NO: 119) | (SEQ ID NO: 120) | (SEQ ID NO: 121) | | |
| rs4290212 | atcttgggctcttgacctga | tcttctccatgggcgttatc | ctgcaagtaaagcaaatcagt | | |
| | (SEQ ID NO: 188) | (SEQ ID NO: 189) | gagttgggcagct | | |
| | | | (SEQ ID NO: 190) | | |
| rs4500466 | ttggccctctgatgaaatgt | acacacccactcctggaaag | Ctcattttgattcctctatc | | |
| | (SEQ ID NO: 191) | (SEQ ID NO: 192) | (SEQ ID NO: 193) | | |
| rs903012 | aaattgcatggcctctcaac | tacggaaggcagctttgtct | agaattaatatttgaagatata | | |
| | (SEQ ID NO: 194) | (SEQ ID NO: 195) | tattagtttatccaatat | | |
| | | | (SEQ ID NO: 196) | | |
| rs4756735 | ccatgatagctctcccaagc | tgcacctgaccttctgctaa | acagtgtcctggacatccacag | | |
| | (SEQ ID NO: 197) | (SEQ ID NO: 198) | gctctcagacaaagataacatt | | |
| | | | at | | |
| | | | (SEQ ID NO: 199) | | |

SNP in Wnt Signalling Genes
Gene = LRP5

| | | | |
|---|---|---|---|
| rs312015 | ggcagagcttcccatgtaga | tggctttagcttgcatttcc | cttagaggccagatcatg |
| | (SEQ ID NO: 122) | (SEQ ID NO: 123) | (SEQ ID NO: 124) |
| rs3736228 | gaccagagcgacgaggag | cagagcccctactcctgtga | actgtcaggaccgctcagacga |
| | (SEQ ID NO: 125) | (SEQ ID NO: 126) | gg |
| | | | (SEQ ID NO: 127) |

Gene = FZD4

| | | | |
|---|---|---|---|
| rs713065 | ccccaaaaaggtcctctaca | atgcacttgtgcggtgatta | agagaaaaaaggtacatcagaa |
| | (SEQ ID NO: 128) | (SEQ ID NO: 129) | acagaaac |
| | | | (SEQ ID NO: 130) |
| rs10898563 | ccatgtccttgtggcctact | ggacctctggaattcccatc | tgtgagccaccacacccagcct |
| | (SEQ ID NO: 131) | (SEQ ID NO: 132) | gggtctctctactt |
| | | | (SEQ ID NO: 133) |
| rs11234890 | ctggtaactgacccctcagc | tgacaatgctttgggttttg | ctttcacattccagacaatgga |
| | (SEQ ID NO: 134) | (SEQ ID NO: 135) | gagtgtttatggttt |
| | | | (SEQ ID NO: 136) |

Gene = DKK2

| | | | |
|---|---|---|---|
| rs17037102 | tggcttcatatttcacatca | attctgccatcccaagtcat | ccagttactgaaagcatcttaa |
| | aga | (SEQ ID NO: 138) | cccctcacatcccggctctgga |
| | (SEQ ID NO: 137) | | tggtactc |
| | | | (SEQ ID NO: 139) |
| rs6827902 | ataaagggaattgggggaaa | gaaaggctattacagggaag | taattccaatattttcaatcat |
| | (SEQ ID NO: 140) | atg | atttactattgtgatgcataaa |
| | | (SEQ ID NO: 141) | aattcagctcagctac |
| | | | (SEQ ID NO: 142) |

Gene = DKK1

| | | | |
|---|---|---|---|
| rs2241529 | tggagaggtggacagataagg | ataagcgctcaaaggctgga | tgcagcgttttcggcgcttcct |
| | (SEQ ID NO: 200) | (SEQ ID NO: 201) | gcaggcgagacagatttgcacg |
| | | | cc |
| | | | (SEQ ID NO: 202) |
| rs1569198 | ccttggatgggtattccaga | cctgaggcacagtctgatga | agtgtcttttgaattattttag |
| | (SEQ ID NO: 203) | (SEQ ID NO: 204) | tgaaacgatgcaggttta |
| | | | (SEQ ID NO: 205) |

TABLE 3
Association Results for SNPs in 5 candidate genes
involved in the Wnt pathway in T2DM and Controls.

| | | N | MAF | N | MAF | X² |
|---|---|---|---|---|---|---|
| Gene | SNP | cases | cases | controls | controls | p value |
| LRP5 | rs312015 | 333 | 38.1% | 218 | 39.0% | 0.65 |
| LRP5 | rs3736228 | 329 | 21.1% | 221 | 21.0% | 0.46 |
| FZD4 | rs10898563 | 332 | 39.9% | 223 | 31.3% | 0.08 |
| FZD4 | rs713065 | 336 | 38.4% | 222 | 38.9% | 0.47 |
| DKK1 | rs2241529 | 337 | 37.8% | 222 | 34.5% | 0.09 |
| DKK1 | rs1569198 | 337 | 49.2% | 221 | 53.9% | 0.07 |
| DKK2 | rs17037102 | 336 | 8.5% | 219 | 9.3% | 0.66 |
| DKK2 | rs6827902 | 336 | 25.4% | 219 | 23.3% | 0.42 |
| DKK3 | rs11022111 | 338 | 16.27% | 223 | 7.62% | 2.25 × 10⁻⁵ |
| DKK3 | rs3206824 | 332 | 44.9% | 222 | 45.7% | 0.97 |

TABLE 4
Genotyping results for rs11022111.

| | | Chinese | | | Utah |
|---|---|---|---|---|---|
| | | South | North | Total | Total |
| Case | CC | 7 | 4 | 11 | 15 |
| | CG | 94 | 90 | 184 | 140 |
| | GG | 226 | 180 | 406 | 256 |
| | total | 327 | 274 | 601 | 411 |
| Control | CC | 1 | 1 | 2 | 6 |
| | CG | 31 | 13 | 44 | 34 |
| | GG | 183 | 80 | 263 | 110 |
| | total | 215 | 94 | 309 | 150 |

TABLE 5
Association analysis of DKK3 (rs11022111) and TCF7L2 (rs7903146) with T2DM.

| | | | | Risk (C) | Risk (CC & | X² | Dominant | Dom. | | |
|---|---|---|---|---|---|---|---|---|---|---|
| | Population | Phenotype | N | % | GG) % | p-value | p-value | OR | CI | PAR |
| DKK3 | Utah | Control | 150 | 15.3% | 26.7% | | | | | |
| | | Type 2 | 411 | 20.7% | 37.7% | 3.52E−02 | 1.50E−02 | 1.57 | 1.10, 2.52 | 7.1% |
| | | Diabetes | | | | | | | | |
| | North Chinese | Control | 94 | 8.0% | 14.9% | | | | | |
| | | Type 2 | 274 | 17.9% | 34.3% | 1.61E−03 | 3.62E−04 | 2.98 | 1.61, 5.55 | 18.8% |
| | | Diabetes | | | | | | | | |
| | South Chinese | Control | 215 | 7.7% | 14.9% | | | | | |
| | | Type 2 | 327 | 16.5% | 30.9% | 1.00E−04 | 2.28E−05 | 2.56 | 1.64, 3.98 | 15.1% |
| | | Diabetes | | | | | | | | |
| | Chinese | Control | 309 | 7.8% | 14.9% | | | | | |
| | | Type 2 | 601 | 17.1% | 32.4% | 9.21E−08 | 1.31E−08 | 2.75 | 1.92, 3.92 | 16.7% |
| | | Diabetes | | | | | | | | |

| | | | | Risk (T) | Trend | OR | | OR | | |
|---|---|---|---|---|---|---|---|---|---|---|
| | Population | Phenotype | N | % | p-value | het | CI | hom | CI | PAR |
| TCF7L2 | Utah | Control | 139 | 28.8% | | | | | | |
| | | Type 2 | 470 | 35.1% | 0.031 | 1.49 | 1.01, 2.21 | 1.79 | 0.79, 4.03 | 10.2% |
| | | Diabetes | | | | | | | | |
| | Chinese | Control | 213 | 8.5% | | | | | | |
| | | Type 2 | 594 | 8.7% | 0.835 | NA | | NA | | |
| | | Diabetes | | | | | | | | |

TABLE 6
Matrices for rs11022111 variants binding sites in the DKK3
promoter region and conservation between mouse and human.

| | | | | Core | Matrix | | |
|---|---|---|---|---|---|---|---|
| rs11022111 | Family/matrix | Position | Strand | Match | Match | Sequence | SEQ ID NO |
| G | V$EGR1.02 | 7-23 | (+) | 1 | 0.901 | tgccgcggGGGCagggg | SEQ ID NO: 143 |
| G | V$SP1.01 | 11-25 | (+) | 1 | 0.911 | gcggGGGCaggggca | SEQ ID NO: 144 |
| C | V$NRF1.01 | 5-21 | (−) | 1 | 0.786 | cctGCGCccgcggcatc | SEQ ID NO: 145 |
| C | V$NRF1.01 | 6-22 | (+) | 0.75 | 0.786 | atgCCGCgggcgcaggg | SEQ ID NO: 146 |
| C | V$NRF1.01 | 11-27 | (−) | 0.75 | 0.81 | cctGCCCctgcgcccgc | SEQ ID NO: 147 |
| C | V$NRF1.01 | 12-28 | (+) | 1 | 0.823 | cggGCGCaggggcaggc | SEQ ID NO: 148 |

Bold indicates the matrix match to binding site.
Uppercase represents the core match.
The variants of rs11022111 are bolded and underlined.

TABLE 7
The effect of DKK3 on Fz- and LRP-dependent β-catenin transcriptional activity

| | | | | | | |
|---|---|---|---|---|---|---|
| ev | Fz1 | Fz2 | Fz3 | Fz4 | Fz5 | Fz6 |

Combined Wnt3a dependent and independent β-catenin activity

| | | | | | | | |
|---|---|---|---|---|---|---|---|
| Dkk3↑ | 5.1 ± 1.2 | 5.8 ± 1.5 | 6.1 ± 2.0 | 1.6 ± 0.6 | 6.2 ± 2.7 | 9.7 ± 3.8 | 2.8 ± 0.8 |
| ev | 11.0 ± 0.3 | 6.0 ± 0.2 | 19.3 ± 7.2 | N.D. | 6.6 ± 1.9 | 5.3 ± 0.6 | 3.4 ± 0.6 |
| Dkk3↓ | 5.1 ± 0.2 | 7.9 ± 2.5 | 3.0 ± 0.8 | 1.4 ± 0.4 | 3.0 ± 1.6 | 4.0 ± 0.9 | 1.1 ± 0.3 |

LRP5

| | | | | | | |
|---|---|---|---|---|---|---|
| Dkk3↑ | 366.8 ± 62.8 | 371.8 ± 50.3 | 34.3 ± 3.0 | 530.5 ± 129.6 | 275.0 ± 85.5 | 459.3 ± 13.7 |
| ev | 555.8 ± 59.2 | 253.8 ± 64.8 | 46.1 ± 4.9 | 497.5 ± 18.6 | 334.9 ± 71.3 | 322.7 ± 23.8 |
| Dkk3↓ | 132.0 ± 13.2 | 100.2 ± 38.2 | 40.9 ± 8.3 | 184.9 ± 61.6 | 189.9 ± 22.3 | 74.8 ± 12.5 |

LRP6

| | | | | | | |
|---|---|---|---|---|---|---|
| Dkk3↑ | 51.7 ± 6.5 | 88.9 ± 3.4 | 46.1 ± 7.4 | 58.6 ± 3.7 | 75.9 ± 14.1 | 69.0 ± 14.8 |
| ev | 61.1 ± 3.2 | 100.9 ± 8.2 | 47.5 ± 17.3 | 54.0 ± 7.6 | 115.1 ± 22.1 | 51.0 ± 6.0 |
| Dkk3↓ | 28.1 ± 5.7 | 3.7 ± 10.3 | 61.7 ± 18.3 | 21.9 ± 3.6 | 31.8 ± 4.1 | 62.0 ± 7.6 |

Wnt3a dependent β-catenin activity

| | | | | | | | |
|---|---|---|---|---|---|---|---|
| Dkk3↑ | 5.1 ± 1.2 | 1.0 ± 0.3 | 1.0 ± 0.3 | 0.5 ± 0.2 | 0.5 ± 0.2 | 1.5 ± 0.6 | 1.1 ± 0.3 |
| ev | 11.0 ± 0.3 | 2.5 ± 0.1 | 6.4 ± 2.4 | N.D. | 2.6 ± 0.7 | 1.4 ± 0.1 | 3.3 ± 0.6 |
| Dkk3↓ | 1.9 ± 0.5 | 1.2 ± 0.4 | 1.3 ± 0.4 | 0.9 ± 0.2 | 1.0 ± 0.5 | 1.0 ± 0.2 | 1.7 ± 0.5 |

LRP5

| | | | | | | |
|---|---|---|---|---|---|---|
| Dkk3↑ | 4.2 ± 0.7 | 3.5 ± 0.2 | 2.2 ± 0.2 | 1.8 ± 0.4 | 1.7 ± 0.5 | 1.2 ± 0.1 |
| ev | 2.7 ± 0.3 | 5.8 ± 1.5 | 2.3 ± 0.3 | 2.4 ± 0.1 | 2.0 ± 0.4 | 1.1 ± 0.1 |
| Dkk3↓ | 2.5 ± 0.2 | 4.4 ± 1.7 | 3.4 ± 0.7 | 1.7 ± 0.6 | 1.2 ± 0.1 | 0.9 ± 0.1 |

LRP6

| | | | | | | |
|---|---|---|---|---|---|---|
| Dkk3↑ | 5.7 ± 0.7 | 4.6 ± 0.2 | 3.1 ± 0.5 | 4.8 ± 0.3 | 5.3 ± 1.0 | 5.4 ± 1.2 |
| ev | 3.6 ± 0.2 | 4.3 ± 0.4 | 3.7 ± 1.3 | 5.8 ± 0.8 | 5.4 ± 1.0 | 7.0 ± 0.8 |
| Dkk3↓ | 4.2 ± 0.9 | 3.0 ± 0.8 | 3.3 ± 1.0 | 4.9 ± 0.8 | 1.6 ± 0.2 | 6.0 ± 0.7 |

| | | | | | |
|---|---|---|---|---|---|
| Fz7 | Fz8 | Fz9 | Fz10 | LRP5 | LRP6 |

Combined Wnt3a dependent and independent β-catenin activity

| | | | | | | |
|---|---|---|---|---|---|---|
| Dkk3↑ | 8.1 ± 0.8 | 9.4 ± 4.5 | 3.9 ± 0.9 | 26.4 ± 7.5 | 414.7 ± 33.7 | 62.9 ± 6.6 |
| ev | 24.2 ± 11.0 | 38.0 ± 7.2 | N.D. | 1.8 ± 0.8 | 80.6 ± 5.3 | 10.8 ± 0.0 |
| Dkk3↓ | 3.8 ± 1.0 | 2.4 ± 1.3 | 6.4 ± 0.7 | 4.8 ± 0.9 | 9.4 ± 0.2 | 5.8 ± 1.1 |

LRP5

| | | | | |
|---|---|---|---|---|
| Dkk3↑ | 105.7 ± 17.1 | 697.7 ± 76.0 | 164.9 ± 15.3 | 77.4 ± 22.7 |
| ev | 147.8 ± 36.9 | 147.8 ± 36.9 | 128.9 ± 13.3 | 58.0 ± 21.8 |
| Dkk3↓ | 43.0 ± 6.2 | 43.0 ± 6.2 | 160.8 ± 25.7 | 28.5 ± 5.8 |

LRP6

| | | | | |
|---|---|---|---|---|
| Dkk3↑ | 64.0 ± 3.0 | 149.4 ± 14.7 | 370.5 ± 34.2 | 9.2 ± 3.7 |
| ev | 48.6 ± 2.5 | 121.5 ± 13.1 | 275.9 ± 13.1 | 12.8 ± 3.1 |
| Dkk3↓ | 19.6 ± 5.7 | 29.8 ± 4.7 | 278.0 ± 4.7 | 9.5 ± 4.0 |

Wnt3a dependent β-catenin activity

| | | | | | | |
|---|---|---|---|---|---|---|
| Dkk3↑ | 0.7 ± 0.1 | 1.3 ± 0.6 | 0.5 ± 0.1 | 7.4 ± 2.1 | 2.8 ± 0.2 | 1.6 ± 0.2 |
| ev | 4.3 ± 1.9 | 13.8 ± 2.6 | N.D. | 1.2 ± 0.6 | 0.9 ± 0.1 | 1.1 ± 0.0 |
| Dkk3↓ | 0.8 ± 0.2 | 3.3 ± 1.8 | 1.9 ± 0.2 | 2.1 ± 0.4 | 2.6 ± 0.1 | 1.5 ± 0.3 |

LRP5

| | | | | |
|---|---|---|---|---|
| Dkk3↑ | 3.1 ± 0.5 | 9.5 ± 1.0 | 3.0 ± 0.3 | 1.3 ± 0.4 |
| ev | 3.4 ± 0.9 | 5.4 ± 1.3 | 2.3 ± 0.2 | 1.1 ± 0.4 |
| Dkk3↓ | 3.0 ± 1.7 | 6.0 ± 0.7 | 3.8 ± 0.4 | 1.2 ± 0.2 |

LRP6

| | | | | |
|---|---|---|---|---|
| Dkk3↑ | 3.9 ± 0.2 | 8.2 ± 0.8 | 4.6 ± 0.4 | 1.1 ± 0.5 |
| ev | 4.7 ± 0.2 | 10.9 ± 1.2 | 11.0 ± 0.2 | 2.0 ± 0.5 |
| Dkk3↓ | 1.2 ± 0.4 | 2.9 ± 0.5 | 5.6 ± 1.7 | 2.1 ± 0.9 |

TABLE 8
Genotype count and association Results for DKK3 in T2DM
and Controls in Han Chinese, Utah and Japanese cohorts

| | Population (rs11022111) | | | | | |
|---|---|---|---|---|---|---|
| | | | | | 3rd Han | |
| | South Han | | North Han | | Chinese | |
| | Chinese | (n = 561) | Chinese | (n = 1027) | Replication | (n = 1440) |
| | Phenotype | | | | | |
| | T2DM | Normal Controls | T2DM | Normal Controls | T2DM | Normal Controls |
| Population Size | 338 | 223 | 534 | 493 | 632 | 808 |
| CC Genotype | 7 | 1 | 10 | 5 | 16 | 7 |
| CG Genotype | 96 | 32 | 164 | 88 | 185 | 150 |
| GG Genotype | 235 | 190 | 360 | 400 | 431 | 651 |
| HWE P value | 0.74 | 0.91 | 0.21 | 1.0 | 0.76 | 0.88 |
| Risk allele Freq | 16.27% | 7.62% | 17.22% | 9.94% | 17.17% | 10.15% |

| | | | |
|---|---|---|---|
| Genotypic P values | 9.77 × 10⁻⁵ | 3.55 × 10⁻⁶ | 1.99 × 10⁻⁷ |
| Trend P values | 1.86 × 10⁻⁵ | 1.04 × 10⁻⁶ | 2.78 × 10⁻⁸ |
| Allelic P values | 2.25 × 10⁻⁵ | 1.62 × 10⁻⁶ | 3.44 × 10⁻⁸ |
| Dominant P values | 2.24 × 10⁻⁵ | 5.50 × 10⁻⁷ | 7.01 × 10⁻⁸ |
| OR_{dom} | 2.43 [1.56, 3.78] | 2.08 [1.56, 2.78] | 1.93 [1.52, 2.46] |

| | Population (rs11022111) | | | | | |
|---|---|---|---|---|---|---|
| | Combined Han | | Utah | | | |
| | Chinese | n = (3028) | Caucasian | (n = 1250) | Japanese | (n = 4652) |
| | Phenotype | | | | | |
| | T2DM | Normal Controls | T2DM | Normal Controls | T2DM | Normal Controls |
| Population Size | 1504 | 1524 | n = 913 | n = 337 | n = 2692 | n = 1960 |
| CC Genotype | 33 | 13 | 40 | 12 | 80 | 44 |
| CG Genotype | 445 | 270 | 300 | 81 | 775 | 490 |
| GG Genotype | 1026 | 1241 | 573 | 244 | 1837 | 1426 |
| HWE P value | 0.16 | 0.93 | 1.0 | 0.29 | 0.99 | 0.97 |
| Risk allele Freq | 16.99% | 9.71% | 20.8% | 15.6% | 17.4% | 14.7% |

| | | | |
|---|---|---|---|
| Genotypic P values | 2.58 × 10⁻¹⁶ | 7.44 × 10⁻³ | p = 3.13 × 10⁻³ |
| Trend P values | 3.60 × 10⁻¹⁷ | 3.74 × 10⁻³ | p = 7.23 × 10⁻⁴ |
| Allelic P values | 8.01 × 10⁻¹⁷ | 3.91 × 10⁻³ | p = 7.16 × 10⁻⁴ |
| Dominant P values | 5.28 × 10⁻¹⁷ | 1.47 × 10⁻³ | p = 8.90 × 10⁻⁴ |
| OR_{dom} | 2.04 [1.73, 2.42] | 1.56 [1.18, 2.05] | 1.23 [1.08, 1.40] |

TABLE 9
Association Results for rs3206824 in DKK3 in T2DM and
Controls in the Utah cohort

| Population | Utah | | |
|---|---|---|---|
| (rs3206824) | Caucasian | | |
| Phenotype | T2DM | Strict Controls | Relaxed Controls |
| Population Size | 863 | 323 | 1080 |
| AA Genotype | 44 | 20 | 65 |
| AG Genotype | 296 | 137 | 420 |
| GG Genotype | 523 | 166 | 595 |
| HWE P value | 0.97 | 0.49 | 0.72 |
| Risk allele Freq | 77.8% | 72.6% | 74.5% |
| Genotypic P | | 1.70 × 10⁻² | 5.0 × 10⁻² |
| values | | | |
| Trend P values | | 8.22 × 10⁻³ | 1.9 × 10⁻² |
| Allelic P values | | 8.52 × 10⁻³ | 2.0 × 10⁻² |
| Recessive P | | 4.22 × 10⁻³ | 1.5 × 10⁻² |
| values | | | |
| ORrec | | 1.46 [1.13, 1.88] | 1.77 [1.49, 2.11] |
| ORhom | | 1.43 [0.821, 2.50] | 1.30 [0.870, 1.94 |
| ORhet | | 0.982 [0.558, 1.73] | 1.04 [0.691, 1.570] |

### J. REFERENCES

Altshuler, D. et al. A haplotype map of the human genome. Nature 437, 1299-1320 (2005).
Bennett, C., Ross, S., Longo, K., Bajnok, L., Hemati, N., Johnson, K., Harrison, S., and MacDougald, O. (2002). Regulation of Wnt signaling during adipogenesis. J Biol Chem 277, 30998-31004.
Brott, B. K., and Sokol, S. Y. (2002). Regulation of Wnt/LRP6 Signaling by Distinct Domains of Dickkopf Proteins. Mol Cell Biol 22, 6100-6110.
Cauchi, S., Meyre, D., Dina, C., Choquet, H., Samson, C., Gallina, S., Balkau, B., Charpentier, G., Pattou, F., Stetsyuk, V., et al. (2006). Transcription Factor TCF7L2 Genetic Study in the French population: Expression in Human beta-cells and Adipose tissue and strong association with type 2 Diabetes. Diabetes 55, 2903-2908.
Chang, Y. C., Chang, T. J., Jiang, Y. D., Kuo, S. S., Lee, K. C., Chiu, K. C., and Chuang, L. M. (2007). Association study of the genetic polymorphisms of the transcription factor 7-like 2 (TCF7L2) gene and type 2 diabetes in the Chinese population. Diabetes epub June 19.
Christodoulides, C., Laudes, M., Cawthorn, W., Schinner, S., Soos, M., O'Rahilly, S., Sethi, J., and Vidal-Puig, A. (2006a). The Wnt antagonist Dickkopf-1 and its receptors are coordinately regulated during early human adipogenesis. J Cell Sci 2006, 2613-2620.
Christodoulides, C., Scarda, A., Granzotto, M., Milan, G., Nora, E. D., Keogh, J., Pergola, G. D., Stirling, H., Pannacciulli, N., Sethi, J., et al. (2006b). Wnt10b mutations in human obesity. Diabetologica 49, 678-684.
Consortium, I. H. (2005). A haplotype map of the human genome. Nature 437, 1241-1242.
Damcott, C., Pollin, T., Reinhardt, L., Ott, S., Shen, H., Silver, K., Mitchell, B., and Shuldiner, A. (2006). Polymorphisms in the Transcription Factor 7-like 2 (TCF7L2) Gene are associated with type 2 diabetes in the Amish. Diabetes 55, 2654-2659.
Diabetes Genetics Initiative of Broad Institute of Harvard and MIT, L. U., and Novartis Institutes of BioMedical Research, Saxena, R., Voight, B., Lyssenko, V., Burtt, N., Bakker, P. d., Chen, H., Roix, J., Kathiresan, S., Daly, M., et al. (2007). Genome-wide association analysis identifies loci for type 2 diabetes and triglyceride levels. Science 316, 1331-1336.
Frayling T M et al. A Common Variant in the FTO Gene Is Associated with Body Mass Index and Predisposes to Childhood and Adult Obesity. Science PMID: 17434869 (2007).
Fujino, T., Asaba, H., Kang, M., Ikeda, Y., Sone, H., Takada, S., Kim, D., ioka, R., Ono, M., Tomoyori, H., et al. (2003). Low-density lipoprotein receptor-related protein 5 (LRP5) is essentail for normal cholesterol metabolism and glucoase-induced insulin secretion. Proc Natl Acad Sci USA 100, 229-234.
Furthauer, M., Thisse, C., and Thisse, B. (1997). A role for FGF-8 in the dorsoventral patterning of the zebrafish gastrula. Development 124, 4253-4264.
Gerdes, J. M., Liu, Y., Zaghloul, N. A., Leitch, C. C., Lawson, S. S., Kato, M., Beachy, P. A., Beales, P. L., Fisher, S., Badano, J. L., and Katsanis, N. (2007). Disruption of ciliary and basal body function perturbs the intracellular Wnt response. submitted.
Grant, S., Thorleifsson, G., Reynisdottir, I., Benediktsson, R., Manolescu, A., Sainz, J., Helgason, A., Stefansson, H., Emilsson, V., Helgadottir, A., et al. (2006). Variant of transcription factor 7-like 2 (TCF7L2) gene confers risk of type 2 diabetes. Nat Gent 38, 320-323.
Heisenberg, C. P., Tada, M., Rauch, G. J., Saude, L., Concha, M. L., Geisler, R., Stemple, D. L., Smith, J. C., and Wilson, S. W. (2000). Silberblick/Wnt11 mediates convergent extension movements during zebrafish gastrulation. Nature 405, 76-81.
Helgason, A., Palsson, S., Thorleifsson, G., Grant, S., Emilsson, V., Gunnarsdottir, S., Adeyemo, A., Chen, Y., Ghen, G., Reynisdottir, I., et al. (2007). Refining the impact of TCF7L2 gene variants of type 2 diabetes and adaptive evolution. Nat Gent 39, 218-225.
Hermann, M., Pirkebner, D., Draxl, A., Berger, P., Untergasser, G., Margreiter, R., and Hengster, P. (2007). Dickkopf-3 is expressed in a subset of adult human pancreatic beta cells. Histochem Cell Biol.
Hey, P. J. et al. Cloning of a novel member of the low-density lipoprotein receptor family. Gene 216, 103-111 (1998).
Hoang, B. H., Kubo, T., Healey, J. H., Yang, R., Nathan, S. S., Kolb, E. A., Mazza, B., Meyers, P. A., and Gorlick, R. (2004). Dickkopf 3 inhibits invasion and motility of Saos-2 osteosarcoma cells by modulating the Wnt-beta-catenin pathway. Cancer Res 64, 2734-2739.
Horikawa, Y., Oda, N., Cox, N. J., Li, X. Q., Orho-Melander, M., Hara, M., Hinokio, Y., Lindner, T. H., Mashima, H., Schwarz, P. E. H., et al. (2000). Genetic variation in the gene encoding calpain-10 is associated with type 2 diabetes mellitus. Nat Genet. 26, 163-175.
Hsieh, S. Y., Hsieh, P. S., Chiu, C. T., and Chen, W. Y. (2004). Dickkopf-3/REIC functions as a suppressor gene of tumor growth. Oncogene 23, 9183-9189.
Humphries, S., Gable, D., Cooper, J., Ireland, H., Stephens, J., Hurel, S., Li, K., Palmen, J., Miller, M., Cappuccio, F., et al. (2006). Common variants in the TCF7L2 gene and predisposition to type 2 diabetes in UK European Whites, Indian Asians and Afro-Carribean men and women. J Mol Med 84, 1005-1014.
Itoh, K., Jacob, J., and Sokol, S. Y. (1998). A role for Xenopus Frizzled 8 in dorsal development. Mech Dev 74, 145-157.
Jho, E. H., Zhang, T., Domon, C., Joo, C. K., Freund, J. N., and Costantini, F. (2002). Wnt/beta-catenin/Tcf signaling induces the transcription of Axin2, a negative regulator of the signaling pathway. Mol Cell Biol 22, 1172-1183.
Katsanis, N., Lupski, J. R., and Beales, P. L. (2001). Exploring the molecular basis of Bardet-Biedl syndrome. Hum Mol Genet. 10, 2293-2299.
Kelly, G. M., Erezyilmaz, D. F., and Moon, R. T. (1995). Induction of a secondary embryonic axis in zebrafish occurs following the overexpression of beta-catenin. Mech Dev 53, 261-273.
Kilian, B., Mansukoski, H., Barbosa, F. C., Ulrich, F., Tada, M., and Heisenberg, C. P. (2003). The role of Ppt/Wnt5 in regulating cell shape and movement during zebrafish gastrulation. Mech Dev 120, 467-476.
Kim, H., Schleiffarth, J., Jessurun, J., Sumanas, S., Petryk, A., Lin, S., and Ekker, S. (2005). Wnt 5 signaling in vertebrate pancreas development. BMC Biol 3.
Korinek, V., Barker, N., Morin, P. J., Wichen, D. v., Weger, R. d., Kinzler, K. W., Vogelstein, B., and Clevers, H. (1997). Constitutive Transcriptional Activation by a beta-Catenin-Tcf Complex in APC−/− Colon Carcinoma. Science 275, 1784-1787.
Krupnik, V. E., Sharp, J. D., Jiang, C., Robison, K., Chickering, T. W., Amaravadi, L., Brown, D. E., Guyot, D., Mays, G., Leiby, K., et al. (1999). Functional and structural diversity of the human Dickkopf gene family. Gene 238, 301-313.
Landesman, Y., and Sokol, S. Y. (1997). Xwnt-2b is a novel axis-inducing Xenopus Wnt, which is expressed in embryonic brain. Mech Dev 63, 199-209.
Lee, A. Y., He, B., You, L., Xu, Z. D., Mazieres, J., Reguart, N., Mikami, I., Batra, S., and Jablons, D. M. (2004). Dickkopf-1 antagonizes Wnt signaling independent of beta-catenin in human mesothelioma. Biochem Biophys Res Commun 323, 1246-1250.
Levin, M. L. The occurrence of lung cancer in man. Acta Unio Int Contra Cancrum 9, 531-41 (1953).
Logan, C. Y., and Nusse, R. (2004). The Wnt signaling pathway in development and disease. Annu Rev Cell Dev Biol 20, 781-810.
Mani, A., Radhakrishnan, J., Wang, H., Mani, A., Mani, M., Nelson-Williams, C., Carew, K., Mane, S., Najmabadi, H., Wu, D., and Lifton, R. (2007). LRP6 mutation in a family with early coronary disease and metabolic risk factors. Science 315, 1278-1282.
Mao, B., Wu, W., Li, Y., Hoppe, D., Stannek, P., Glinka, A., and Niehrs, C. (2001). LDL-receptor related protein 6 is a receptor for Dickkopf proteins. Nature 411, 321-325.
McMahon, A. P. (1992). The Wnt family of developmental regulators. Trends Genet. 8, 1-5.
Meyre, D., Bouatia-Naji, N., Tounian, A., Samson, C., Lecoeur, C., Vatin, V., Ghoussaini, M., Wachter, C., Hercberg, S., Charpentier, G., et al. (2005). Variants of ENPP1 are associated with childhood and adult obesity and increase the risk of glucose intolerance and type 2 diabetes. Nat Genet. 37, 863-867.
Munoz, J., Lok, K., Gower, B., Fernandez, J., Hunter, G., Lara-Castro, C., Luca, M. D., and Garvey, W. (2006). Polymorphism in the Transcription Factor 7-like2 (TCF7L2) gene is Associated with reduced insulin Secretion in nondiabetic women. Diabetes 55, 3630-3634.
Ng, M. C., Tam, C. H., Lam, V. K., So, W. Y., Ma, R. C., and Chan, J. C. (2007). Replication and identification of novel variants at TFC7L2 associated with type 2 diabetes in Hong Kong Chinese. J Endocrinol Metab epub July 3.
Niehrs, C. (2006). Function and biological roles of the Dickkopf family of Wnt modulators. Oncogene 25, 7649-7681.
Permutt, M. A., Wasson, J., and Cox, N. (2005). Genetic epidemiology of diabetes. J Clin Invest 115, 1431-1439.
Ramel, M.-C., Buckles, G. R., Baker, K. D., and Lekven, A. C. (2005). WNT8 and BMP2B co-regulate non-axial mesodermal patterning during zebrafish gastrulation. Dev Biol 287, 237-248.
Robitaille, J., MacDonald, M., Kavkas, A., Sheldahl, L., Zeisler, J., Dube, M., Zhang, L., Singaraja, R., Guernsey, D., Zheng, B., et al. (2002). Mutant frizzled-4 disrupts angiogenesis in familial exudative vitreoretinopathy. Nat Gent 32, 326-330.
Ross, A. J., May-Simera, H., Eichers, E. R., Kai, M., Hill, J., Jagger, D. J., Leitch, C. C., Chapple, J. P., Munro, P. M., Fisher, S., et al. (2005). Disruption of Bardet-Biedl syndrome ciliary proteins perturbs planar cell polarity in vertebrates. Nature Genet. 37, 1135-1140.
Ross, S., Hemati, N., Longo, K., Bennett, C., Lucas, P., Erickson, R., and MacDougald, O. (2000). Inhibition of Adipogenesis by Wnt Signaling. Science 289, 950-953.
Rulifson, I. C., Karnik, S. K., Heiser, P. W., ten Berge, D., Chen, H., Gu, X., Taketo, M. M., Nusse, R., Hebrok, M., and Kim, S. K. (2007). Wnt signaling regulates pancreatic beta cell proliferation. Proc Natl Acad Sci USA 104, 6247-6252.
Saxena, R., Gianniny, L., Burtt, N., Lyssenko, V., Giuducci, C., Sjoegren, M., Florez, J., Almgren, P., Isomaa, B., Orho-Melander, M., et al. (2006). Common Single Nucleotide polymorphisms in TCF7L2 are reproducibly associated with Type 2 Diabetes and reduce the insulin response to glucose in nondiabetic individuals. Diabetes 55, 289-2895.
Scott, L., Mohlke, K., Bonnycastle, L., Willer, C., Li, Y., Duren, W., Erdos, M., Stringham, H., Chines, P., JAckson, A., et al. (2007). A genome-wide association study of type 2 diabetes in Finns detects multiple susceptibility variants. Science 316, 1341-1345.
Sharma, K., McCue, P. & Dunn, S. R. Diabetic kidney disease in the db/db mouse. Am J Physiol Renal Physiol 284, F1138-44 (2003).
Silander, K., Mohlke, K. L., Scott, L. J., Peck, E. C., Hollstein, P., Skol, A. D., Jackson, A. U., Deloukas, P., Hunt, S., Stavrides, G., et al. (2004). Genetic variation near the hepatocyte nuclear factor-4 alpha gene predicts susceptibility to type 2 diabetes. Diabetes 53, 1141-1149.
Sladek, R., Rocheleau, G., Rung, J., Dina, C., Shen, L., Serre, D., Boutin, P., Vincent, D., Belisle, A., Hadjadj, S., et al. (2007). A genome-wide association study identifies novel risk loci for type2 diabetes. Nature 445, 881-885.
Thisse, C., Thisse, B., Schilling, T. F. & Postlethwait, J. H. Structure of the Zebrafish Snaill Gene and Its Expression in Wild-Type, Spadetail and No Tail Mutant Embryos. Development 119, 1203-1215 (1993).
Toomes, C., Bottomley, H., Jackson, R., Towns, K., Scott, S., Mackey, D., Craig, J., Jiang, L., Yang, Z., Trembath, R., et al. (2004). Mutations in LRP5 or FZD4 underlie the common familial exudative vitreoretinopathy locus on chromosome q11. Am J Hum Gent 74, 721-730.
Veeman, M. T., Slusarski, D. C., Kaykas, A., Louie, S. H., and Moon, R. T. (2003). Zebrafish prickle, a modulator of noncanonical Wnt/Fz signaling, regulates gastrulation movements. Curr Biol 13, 680-685.
Weedon, M. N., Owen, K. R., Shields, B., Hitman, G., Walker, M., McCarthy, M. I., Love-Gregory, L. D., Permutt, M. A., Hattersley, A. T., and Frayling, T. M. (2004). Common variants of the hepatocyte nuclear factor-4 alpha P2 promoter are associated with type 2 diabetes in the UK population. Diabetes 53, 3002-3006.
Wells, J. et al. Wnt/beta-catenin signaling is required for the development of the exocrine pancreas. BMC Dev Biol 7 (2007).
Willert, K. et al. Wnt proteins are lipid-modified and can act as stem cell growth factors. Nature 423, 448-52 (2003).
Xu, Q., Wang, Y., Dabdoub, A., Smallwood, P., Williams, J., Woods, C., Kelley, M., Jiang, L., Tasman, W., Zhang, K., and Nathans, J. (2004). Vascular development in the retina and inner ear: control by Norrin and Frizzled-4, a high affinity ligand-receptor pair. Cell 116, 883-895.
Yang, Z. et al. A variant of the HTRA1 gene increases susceptibility to age-related macular degeneration. Science 314, 992-3 (2006).
Zeggini, E., Weedon, M., Lindgren, C., Frayling, T., Elliott, K., Lango, H., Timpson, N., Pewrry, J., Rayner, N., Freathy, R., et al. (2007). Replication of genome-wide association signals in UK samples reveals risk loci for type 2 diabetes. Science 316, 1336-1341.

What is claimed is:

1. A method of identifying a subject at risk for developing type 2 diabetes, comprising detecting in a sample of nucleic acid from a cystidine at nucleotide position 11987669 on chromosome 11.

2. The method of claim 1, wherein the cystidine is detected by a process comprising
a) providing a probe that hybridizes under stringent conditions to an oligonucleotide consisting of SEQ ID NO:1 but does not hybridizes under stringent conditions to an oligonucleotide consisting of SEQ ID NO:2, and
b) detecting hybridization of said probe to the nucleic acid sample.

3. The method of claim 1, wherein the cystidine is detected by gene sequencing.

4. The method of claim 1, wherein the cystidine is detected by allele specific hybridization.

5. The method of claim 1, wherein the identified subject is monitored for levels of DKK3.

6. The method of claim 1, wherein a treatment protocol is chosen for the subject based on the detection of cystidine at nucleotide position 11987669 on chromosome 11.

7. The method of claim 1, wherein the subject has a family history of T2DM.

8. A method, comprising administering a therapeutically effective amount of an agent that increases DKK3 activity to a subject identified at having or at risk for developing type 2 diabetes.

9. A method, comprising administering a therapeutically effective amount of an agent that activates the Wnt pathway to a subject identified at having or at risk for developing type 2 diabetes.

10. The method of claim 7 or 9, wherein the agent is a peptide comprising DKK3 or a fragment thereof.

11. The method of claim 10, wherein the peptide comprises SEQ ID NO:160 or a fragment thereof of at least about 20 amino acids in length that can bind a Wnt-related receptor and modulate the Wnt pathway.

12. The method of claim 7 or 9, wherein the agent is a nucleic acid encoding DKK3 or a fragment thereof.

13. The method of claim 9, wherein the agent is an FZ5 agonist.

14. The method of claim 13, wherein the agent is a peptide comprising FZ5 or a fragment thereof.

15. The method of claim 14, wherein the agent is a peptide comprising SEQ ID NO:163 or a fragment thereof of at least about 20 amino acids in length that can modulate the Wnt signaling pathway.

16. The method of claim 13, wherein the agent is a nucleic acid encoding FZ5 or a fragment thereof.

17. The method of claim 9, wherein the agent is an FZ7 agonist.

18. The method of claim 17, wherein the agent is a peptide comprising FZ7 or a fragment thereof.

19. The method of claim 18, wherein the agent is a peptide comprising SEQ ID NO:184 or a fragment thereof of at least about 20 amino acids in length that can modulate the Wnt signaling pathway.

20. The method of claim 17, wherein the agent is a nucleic acid encoding FZ7 or a fragment thereof.

21. The method of claim 9, wherein the agent is an FZ8 agonist.

22. The method of claim 21, wherein the agent is a peptide comprising FZ5 or a fragment thereof.

23. The method of claim 22, wherein the agent is a peptide comprising SEQ ID NO:166 or a fragment thereof of at least about 20 amino acids in length that can modulate the Wnt signaling pathway.

24. The method of claim 21, wherein the agent is a nucleic acid encoding FZ7 or a fragment thereof.

25. The method of claim 9, wherein the agent is an LRP6 agonist.

26. The method of claim 25, wherein the agent is a peptide comprising FZ5 or a fragment thereof.

27. The method of claim 26, wherein the agent is a peptide comprising SEQ ID NO:172 or a fragment thereof of at least about 20 amino acids in length that can modulate the Wnt signaling pathway.

28. The method of claim 25, wherein the agent is a nucleic acid encoding LRP6 or a fragment thereof.

29. A method of identifying a composition for treating or preventing type II diabetes mellitus, comprising contacting a candidate agent to a cell comprising a nucleic acid encoding DKK3 operably linked to an expression control sequence and detecting DKK3 levels and/or activity in the cell, wherein detection of an increase in DKK3 levels and/or activity in the cell compared to a reference control identifies a composition for treating or preventing type II diabetes mellitus.

30. The method of claim 29, wherein the detection is part of a high throughput assay.


SEQUENCE LISTING (205 sequences)

SEQ ID NO: 1 - DNA, 20 bp, Artificial Sequence
ccaccctgctgcttttactc

SEQ ID NO: 2 - DNA, 20 bp, Artificial Sequence
  Description of Artificial Sequence:  note = Synthetic Construct
ctcagtctgggaccttctgc

SEQ ID NO: 3 - DNA, 20 bp, Artificial Sequence
  Description of Artificial Sequence:  note = Synthetic Construct
gtgaaggtcggtgtgaacgg

SEQ ID NO: 4 - DNA, 20 bp, Artificial Sequence
  Description of Artificial Sequence:  note = Synthetic Construct
gccgttgaatttgccgtgag

SEQ ID NO: 5 - DNA, 30 bp, Artificial Sequence
  Description of Artificial Sequence:  note = Synthetic Construct
gaagatcccgttggggtttcaagctggaga

SEQ ID NO: 6 - DNA, 31 bp, Artificial Sequence
  Description of Artificial Sequence:  note = Synthetic Construct
cccaagcttgggtccgctctgcgcccgcagc

SEQ ID NO: 7 - DNA, 29 bp, Artificial Sequence
  Description of Artificial Sequence:  note = Synthetic Construct
gaagatcttcgcaggggcaggcagtgaag

SEQ ID NO: 8 - DNA, 25 bp, Artificial Sequence
  Description of Artificial Sequence:  note = Synthetic Construct
agaggctgaatccgagcagaaacat

SEQ ID NO: 9 - DNA, 25 bp, Artificial Sequence
  Description of Artificial Sequence:  note = Synthetic Construct
cctcttacctcagttacaatttata

SEQ ID NO: 10 - DNA, 20 bp, Artificial Sequence
  Description of Artificial Sequence:  note = Synthetic Construct
cgtagaccccaaaacaggaa

SEQ ID NO: 11 - DNA, 20 bp, Artificial Sequence
  Description of Artificial Sequence:  note = Synthetic Construct
tcctgtcgtgattgggtaca

SEQ ID NO: 12 - DNA, 20 bp, Artificial Sequence
  Description of Artificial Sequence:  note = Synthetic Construct
atatgcgactgcgaacactg

SEQ ID NO: 13 - DNA, 23 bp, Artificial Sequence
  Description of Artificial Sequence:  note = Synthetic Construct
gcagttgaagtgatttatgcttg

SEQ ID NO: 14 - DNA, 20 bp, Artificial Sequence
  Description of Artificial Sequence:  note = Synthetic Construct
aggtgtcatggactgttgcc

SEQ ID NO: 15 - DNA, 20 bp, Artificial Sequence
  Description of Artificial Sequence:  note = Synthetic Construct
agcgcaatacatgcaaggtt

SEQ ID NO: 16 - DNA, 22 bp, Artificial Sequence
  Description of Artificial Sequence:  note = Synthetic Construct
tgtctacctcaactgtggcttc

SEQ ID NO: 17 - DNA, 18 bp, Artificial Sequence
  Description of Artificial Sequence:  note = Synthetic Construct
tatcatgatcattgaaca

SEQ ID NO: 18 - DNA, 24 bp, Artificial Sequence
  Description of Artificial Sequence:  note = Synthetic Construct
tcttcacccagtctacactaatgg

SEQ ID NO: 19 - DNA, 20 bp, Artificial Sequence
  Description of Artificial Sequence:  note = Synthetic Construct
atcgccagggattaaagagg

SEQ ID NO: 20 - DNA, 24 bp, Artificial Sequence
  Description of Artificial Sequence:  note = Synthetic Construct
atacactatcagaaccaagatatt

SEQ ID NO: 21 - DNA, 20 bp, Artificial Sequence
  Description of Artificial Sequence:  note = Synthetic Construct
ttggggcatacagagtttcc

SEQ ID NO: 22 - DNA, 20 bp, Artificial Sequence
  Description of Artificial Sequence:  note = Synthetic Construct
aaggaaaagcctgtcccagt

SEQ ID NO: 23 - DNA, 30 bp, Artificial Sequence
  Description of Artificial Sequence:  note = Synthetic Construct
catataagttaggtgaagacttgactctta

SEQ ID NO: 24 - DNA, 20 bp, Artificial Sequence
  Description of Artificial Sequence:  note = Synthetic Construct
ggtaagacaggctgcagagg

SEQ ID NO: 25 - DNA, 20 bp, Artificial Sequence
  Description of Artificial Sequence:  note = Synthetic Construct
gagttgccgataggtcttgc

SEQ ID NO: 26 - DNA, 20 bp, Artificial Sequence
  Description of Artificial Sequence:  note = Synthetic Construct
ggaaatggtgaatgctgttg

SEQ ID NO: 27 - DNA, 22 bp, Artificial Sequence
  Description of Artificial Sequence:  note = Synthetic Construct
aagaggattaggcttcaaatcg

SEQ ID NO: 28 - DNA, 56 bp, Artificial Sequence
  Description of Artificial Sequence:  note = Synthetic Construct
tggggaagacagtgttcatgacaggtttataggtccgcttccaagagaaggttctg

SEQ ID NO: 29 - DNA, 20 bp, Artificial Sequence
  Description of Artificial Sequence:  note = Synthetic Construct
gcagggaaattttgaaacca

SEQ ID NO: 30 - DNA, 20 bp, Artificial Sequence
  Description of Artificial Sequence:  note = Synthetic Construct
gcaaagttccatgccaaaat

SEQ ID NO: 31 - DNA, 20 bp, Artificial Sequence
  Description of Artificial Sequence:  note = Synthetic Construct
cctctgcttccaggttcaag

SEQ ID NO: 32 - DNA, 20 bp, Artificial Sequence
  Description of Artificial Sequence:  note = Synthetic Construct
atcctttggcattgctcatc

SEQ ID NO: 33 - DNA, 40 bp, Artificial Sequence
  Description of Artificial Sequence:  note = Synthetic Construct
caaagtgctgggattacaggcctgagccatcacgcctggc

SEQ ID NO: 34 - DNA, 19 bp, Artificial Sequence
  Description of Artificial Sequence:  note = Synthetic Construct
ccctcctcttctgcctcag

SEQ ID NO: 35 - DNA, 20 bp, Artificial Sequence
  Description of Artificial Sequence:  note = Synthetic Construct
agactaaggccagcggtgat

SEQ ID NO: 36 - DNA, 28 bp, Artificial Sequence
  Description of Artificial Sequence:  note = Synthetic Construct
agccatctccttcactgcctgcccctgc

SEQ ID NO: 37 - DNA, 19 bp, Artificial Sequence
  Description of Artificial Sequence:  note = Synthetic Construct
ccctcctcttctgcctcag

SEQ ID NO: 38 - DNA, 20 bp, Artificial Sequence
  Description of Artificial Sequence:  note = Synthetic Construct
agctgcaagtgcgttatcct

SEQ ID NO: 39 - DNA, 36 bp, Artificial Sequence
  Description of Artificial Sequence:  note = Synthetic Construct
gcctccaggcttgcaacagcccaccgaggttggggg

SEQ ID NO: 40 - DNA, 20 bp, Artificial Sequence
  Description of Artificial Sequence:  note = Synthetic Construct
gggaaaagacaccaggatca

SEQ ID NO: 41 - DNA, 20 bp, Artificial Sequence
  Description of Artificial Sequence:  note = Synthetic Construct
ctcacccacccacagaaagt

SEQ ID NO: 42 - DNA, 18 bp, Artificial Sequence
  Description of Artificial Sequence:  note = Synthetic Construct
agccactaatgtaatgga

SEQ ID NO: 43 - DNA, 20 bp, Artificial Sequence
  Description of Artificial Sequence:  note = Synthetic Construct
accccaatgtttccacttga

SEQ ID NO: 44 - DNA, 20 bp, Artificial Sequence
  Description of Artificial Sequence:  note = Synthetic Construct
tgggctacagggtggtatgt

SEQ ID NO: 45 - DNA, 42 bp, Artificial Sequence
  Description of Artificial Sequence:  note = Synthetic Construct
ccctggtcccaccagcctgactccttttccccaattagcact

SEQ ID NO: 46 - DNA, 20 bp, Artificial Sequence
  Description of Artificial Sequence:  note = Synthetic Construct
tcctcccagctttattgagg

SEQ ID NO: 47 - DNA, 20 bp, Artificial Sequence
  Description of Artificial Sequence:  note = Synthetic Construct
atggggagatgttggtgaaa

SEQ ID NO: 48 - DNA, 24 bp, Artificial Sequence
  Description of Artificial Sequence:  note = Synthetic Construct
gtagttaccatttttttgtgtgtg

SEQ ID NO: 49 - DNA, 19 bp, Artificial Sequence
  Description of Artificial Sequence:  note = Synthetic Construct
gtaggggagctgcgttttc

SEQ ID NO: 50 - DNA, 20 bp, Artificial Sequence
  Description of Artificial Sequence:  note = Synthetic Construct
tccagactttttggcaagga

SEQ ID NO: 51 - DNA, 27 bp, Artificial Sequence
  Description of Artificial Sequence:  note = Synthetic Construct
tagctgagagccgggccgggcttgact

SEQ ID NO: 52 - DNA, 19 bp, Artificial Sequence
  Description of Artificial Sequence:  note = Synthetic Construct
gtaggggagctgcgttttc

SEQ ID NO: 53 - DNA, 20 bp, Artificial Sequence
  Description of Artificial Sequence:  note = Synthetic Construct
cgtgtcctccatcagttcct

SEQ ID NO: 54 - DNA, 20 bp, Artificial Sequence
  Description of Artificial Sequence:  note = Synthetic Construct
ttggggccaccctgctgtgc

SEQ ID NO: 55 - DNA, 20 bp, Artificial Sequence
  Description of Artificial Sequence:  note = Synthetic Construct
cggagatcacaggttgaagc

SEQ ID NO: 56 - DNA, 20 bp, Artificial Sequence
  Description of Artificial Sequence:  note = Synthetic Construct
gacgttcttttcccctctcc

SEQ ID NO: 57 - DNA, 48 bp, Artificial Sequence
  Description of Artificial Sequence:  note = Synthetic Construct
tgatggagaaggaagttccctggaagagggcaggggagcgcctctcca

SEQ ID NO: 58 - DNA, 20 bp, Artificial Sequence
  Description of Artificial Sequence:  note = Synthetic Construct
tgccacctgtgtctgtatgc

SEQ ID NO: 59 - DNA, 20 bp, Artificial Sequence
  Description of Artificial Sequence:  note = Synthetic Construct
ggaaaggactttgcagtgga

SEQ ID NO: 60 - DNA, 54 bp, Artificial Sequence
  Description of Artificial Sequence:  note = Synthetic Construct
ggagtgtagtggcatgatctcggttcactgcagccttgatctctggggctcatt

SEQ ID NO: 61 - DNA, 20 bp, Artificial Sequence
  Description of Artificial Sequence:  note = Synthetic Construct
atatcgaactgccccaagtg

SEQ ID NO: 62 - DNA, 20 bp, Artificial Sequence
  Description of Artificial Sequence:  note = Synthetic Construct
cagatctggtttgggctcat

SEQ ID NO: 63 - DNA, 36 bp, Artificial Sequence
  Description of Artificial Sequence:  note = Synthetic Construct
aaactacaaacgaaaaaaagaaaggaaagaaaagaa

SEQ ID NO: 64 - DNA, 20 bp, Artificial Sequence
  Description of Artificial Sequence:  note = Synthetic Construct
tttccaagccaacctctgtc

SEQ ID NO: 65 - DNA, 20 bp, Artificial Sequence
  Description of Artificial Sequence:  note = Synthetic Construct
gggacgcatacaaatctggt

SEQ ID NO: 66 - DNA, 20 bp, Artificial Sequence
  Description of Artificial Sequence:  note = Synthetic Construct
caggagctgcaagtgcgtta

SEQ ID NO: 67 - DNA, 20 bp, Artificial Sequence
  Description of Artificial Sequence:  note = Synthetic Construct
gccactcacctctctggaag

SEQ ID NO: 68 - DNA, 20 bp, Artificial Sequence
  Description of Artificial Sequence:  note = Synthetic Construct
ggaaaaagggaacagcacaa

SEQ ID NO: 69 - DNA, 34 bp, Artificial Sequence
  Description of Artificial Sequence:  note = Synthetic Construct
gatccatgagtcctcccggtgccatttttccagc

SEQ ID NO: 70 - DNA, 20 bp, Artificial Sequence
  Description of Artificial Sequence:  note = Synthetic Construct
gaagagcttgccaacaaagg

SEQ ID NO: 71 - DNA, 20 bp, Artificial Sequence
  Description of Artificial Sequence:  note = Synthetic Construct
tgtttctcttcccctcatgg

SEQ ID NO: 72 - DNA, 46 bp, Artificial Sequence
  Description of Artificial Sequence:  note = Synthetic Construct
tcactaaccagagtggtaactgagggcaaggtctcttgcttttaag

SEQ ID NO: 73 - DNA, 20 bp, Artificial Sequence
  Description of Artificial Sequence:  note = Synthetic Construct
gggactccatcctgaagtga

SEQ ID NO: 74 - DNA, 20 bp, Artificial Sequence
  Description of Artificial Sequence:  note = Synthetic Construct
tcctcgggacaacaaagaag

SEQ ID NO: 75 - DNA, 40 bp, Artificial Sequence
  Description of Artificial Sequence:  note = Synthetic Construct
gaatctgcccagaggtcacccagcttgactggactggagc

SEQ ID NO: 76 - DNA, 20 bp, Artificial Sequence
  Description of Artificial Sequence:  note = Synthetic Construct
cctgagagtgagggttaggg

SEQ ID NO: 77 - DNA, 20 bp, Artificial Sequence
  Description of Artificial Sequence:  note = Synthetic Construct
tgggaggttatgctccattt

SEQ ID NO: 78 - DNA, 46 bp, Artificial Sequence
  Description of Artificial Sequence:  note = Synthetic Construct
aggtccagaagccggctggcggggtcatggcaaagctcgccctcca

SEQ ID NO: 79 - DNA, 20 bp, Artificial Sequence
  Description of Artificial Sequence:  note = Synthetic Construct
ggccatactacccaaagcaa

SEQ ID NO: 80 - DNA, 21 bp, Artificial Sequence
  Description of Artificial Sequence:  note = Synthetic Construct
tgggctctttattttgttcca

SEQ ID NO: 81 - DNA, 48 bp, Artificial Sequence
  Description of Artificial Sequence:  note = Synthetic Construct
caagagtctgaatagccaacacaattttaagcaaaaagaataaagctg

SEQ ID NO: 82 - DNA, 20 bp, Artificial Sequence
  Description of Artificial Sequence:  note = Synthetic Construct
gaccaagatggggagatcct

SEQ ID NO: 83 - DNA, 20 bp, Artificial Sequence
  Description of Artificial Sequence:  note = Synthetic Construct
taggaagaagcctggtcagc

SEQ ID NO: 84 - DNA, 38 bp, Artificial Sequence
  Description of Artificial Sequence:  note = Synthetic Construct
aggacctggagaggagcctgactgaagagatggcgctg

SEQ ID NO: 85 - DNA, 20 bp, Artificial Sequence
  Description of Artificial Sequence:  note = Synthetic Construct
gatgagcaatgccaaaggat

SEQ ID NO: 86 - DNA, 20 bp, Artificial Sequence
  Description of Artificial Sequence:  note = Synthetic Construct
cattcatttgctggttgtgc

SEQ ID NO: 87 - DNA, 20 bp, Artificial Sequence
  Description of Artificial Sequence:  note = Synthetic Construct
agaagtgccacacaaaagct

SEQ ID NO: 88 - DNA, 20 bp, Artificial Sequence
  Description of Artificial Sequence:  note = Synthetic Construct
ccgggaacacagcatagatt

SEQ ID NO: 89 - DNA, 20 bp, Artificial Sequence
  Description of Artificial Sequence:  note = Synthetic Construct
cttgtcctctcctcctgcac

SEQ ID NO: 90 - DNA, 28 bp, Artificial Sequence
  Description of Artificial Sequence:  note = Synthetic Construct
tcagagtctttgattttccagtttagag

SEQ ID NO: 91 - DNA, 20 bp, Artificial Sequence
  Description of Artificial Sequence:  note = Synthetic Construct
cctggcgaagttaccagatt

SEQ ID NO: 92 - DNA, 20 bp, Artificial Sequence
  Description of Artificial Sequence:  note = Synthetic Construct
cacccccagaaagctaatga

SEQ ID NO: 93 - DNA, 28 bp, Artificial Sequence
  Description of Artificial Sequence:  note = Synthetic Construct
actgcctttctctgcagcactccccaca

SEQ ID NO: 94 - DNA, 20 bp, Artificial Sequence
  Description of Artificial Sequence:  note = Synthetic Construct
agcaaagtccagcctgaaaa

SEQ ID NO: 95 - DNA, 20 bp, Artificial Sequence
  Description of Artificial Sequence:  note = Synthetic Construct
cccaccaatgctaggaggta

SEQ ID NO: 96 - DNA, 36 bp, Artificial Sequence
  Description of Artificial Sequence:  note = Synthetic Construct
aattctgcttctttattataagttatttgttatttc

SEQ ID NO: 97 - DNA, 20 bp, Artificial Sequence
  Description of Artificial Sequence:  note = Synthetic Construct
agggatctggcatctcattg

SEQ ID NO: 98 - DNA, 20 bp, Artificial Sequence
  Description of Artificial Sequence:  note = Synthetic Construct
tgtttgactggggtgtctga

SEQ ID NO: 99 - DNA, 30 bp, Artificial Sequence
  Description of Artificial Sequence:  note = Synthetic Construct
atcaatttctatgtttattcaaaagaaact

SEQ ID NO: 100 - DNA, 20 bp, Artificial Sequence
  Description of Artificial Sequence:  note = Synthetic Construct
gagtcagcagtgtgggtgaa

SEQ ID NO: 101 - DNA, 20 bp, Artificial Sequence
  Description of Artificial Sequence:  note = Synthetic Construct
ggttccattgcaactgtcct

SEQ ID NO: 102 - DNA, 34 bp, Artificial Sequence
  Description of Artificial Sequence:  note = Synthetic Construct
tgtagggctgagacaggcttttgcgcccacacct

SEQ ID NO: 103 - DNA, 20 bp, Artificial Sequence
  Description of Artificial Sequence:  note = Synthetic Construct
ggactgagaagtgcctttgc

SEQ ID NO: 104 - DNA, 20 bp, Artificial Sequence
  Description of Artificial Sequence:  note = Synthetic Construct
ctgatgggctgagtcacaga

SEQ ID NO: 105 - DNA, 20 bp, Artificial Sequence
  Description of Artificial Sequence:  note = Synthetic Construct
aaaacgtagcaggtactcaa

SEQ ID NO: 106 - DNA, 20 bp, Artificial Sequence
  Description of Artificial Sequence:  note = Synthetic Construct
ttctttctggggaaggaggt

SEQ ID NO: 107 - DNA, 20 bp, Artificial Sequence
  Description of Artificial Sequence:  note = Synthetic Construct
gatggggtgtcagacttgct

SEQ ID NO: 108 - DNA, 20 bp, Artificial Sequence
  Description of Artificial Sequence:  note = Synthetic Construct
ccacctactccagcaaccat

SEQ ID NO: 109 - DNA, 20 bp, Artificial Sequence
  Description of Artificial Sequence:  note = Synthetic Construct
gtgctctgggtggagaagag

SEQ ID NO: 110 - DNA, 20 bp, Artificial Sequence
  Description of Artificial Sequence:  note = Synthetic Construct
ctcttctccacccagagcac

SEQ ID NO: 111 - DNA, 20 bp, Artificial Sequence
  Description of Artificial Sequence:  note = Synthetic Construct
ctggagagcaaccaagaagg

SEQ ID NO: 112 - DNA, 28 bp, Artificial Sequence
  Description of Artificial Sequence:  note = Synthetic Construct
cacagccgtctagagaaaaaaggcctgt

SEQ ID NO: 113 - DNA, 20 bp, Artificial Sequence
  Description of Artificial Sequence:  note = Synthetic Construct
gggactccatcctgaagtga

SEQ ID NO: 114 - DNA, 20 bp, Artificial Sequence
  Description of Artificial Sequence:  note = Synthetic Construct
tcctcgggacaacaaagaag

SEQ ID NO: 115 - DNA, 46 bp, Artificial Sequence
  Description of Artificial Sequence:  note = Synthetic Construct
gtgacctctgggcagattctatccttaagtctcagtttccacatct

SEQ ID NO: 116 - DNA, 20 bp, Artificial Sequence
  Description of Artificial Sequence:  note = Synthetic Construct
tgatccagtcaccatccaaa

SEQ ID NO: 117 - DNA, 20 bp, Artificial Sequence
  Description of Artificial Sequence:  note = Synthetic Construct
ctgggaaaacaatccagcac

SEQ ID NO: 118 - DNA, 42 bp, Artificial Sequence
  Description of Artificial Sequence:  note = Synthetic Construct
cataggcatttagaccagggcacccagtgacacatcttttct

SEQ ID NO: 119 - DNA, 22 bp, Artificial Sequence
  Description of Artificial Sequence:  note = Synthetic Construct
gatgaatacatggtggcaacag

SEQ ID NO: 120 - DNA, 22 bp, Artificial Sequence
  Description of Artificial Sequence:  note = Synthetic Construct
atgactgagcgtagcatacagg

SEQ ID NO: 121 - DNA, 31 bp, Artificial Sequence
  Description of Artificial Sequence:  note = Synthetic Construct
tggcgtagagttcagtgttcgcagtcgcata

SEQ ID NO: 122 - DNA, 20 bp, Artificial Sequence
  Description of Artificial Sequence:  note = Synthetic Construct
ggcagagcttcccatgtaga

SEQ ID NO: 123 - DNA, 20 bp, Artificial Sequence
  Description of Artificial Sequence:  note = Synthetic Construct
tggctttagcttgcatttcc

SEQ ID NO: 124 - DNA, 18 bp, Artificial Sequence
  Description of Artificial Sequence:  note = Synthetic Construct
cttagaggccagatcatg

SEQ ID NO: 125 - DNA, 18 bp, Artificial Sequence
  Description of Artificial Sequence:  note = Synthetic Construct
gaccagagcgacgaggag

SEQ ID NO: 126 - DNA, 20 bp, Artificial Sequence
  Description of Artificial Sequence:  note = Synthetic Construct
cagagcccctactcctgtga

SEQ ID NO: 127 - DNA, 24 bp, Artificial Sequence
  Description of Artificial Sequence:  note = Synthetic Construct
actgtcaggaccgctcagacgagg

SEQ ID NO: 128 - DNA, 20 bp, Artificial Sequence
  Description of Artificial Sequence:  note = Synthetic Construct
ccccaaaaaggtcctctaca

SEQ ID NO: 129 - DNA, 20 bp, Artificial Sequence
  Description of Artificial Sequence:  note = Synthetic Construct
atgcacttgtgcggtgatta

SEQ ID NO: 130 - DNA, 30 bp, Artificial Sequence
  Description of Artificial Sequence:  note = Synthetic Construct
agagaaaaaaggtacatcagaaacagaaac

SEQ ID NO: 131 - DNA, 20 bp, Artificial Sequence
  Description of Artificial Sequence:  note = Synthetic Construct
ccatgtccttgtggcctact

SEQ ID NO: 132 - DNA, 20 bp, Artificial Sequence
  Description of Artificial Sequence:  note = Synthetic Construct
ggacctctggaattcccatc

SEQ ID NO: 133 - DNA, 36 bp, Artificial Sequence
  Description of Artificial Sequence:  note = Synthetic Construct
tgtgagccaccacacccagcctgggtctctctactt

SEQ ID NO: 134 - DNA, 20 bp, Artificial Sequence
  Description of Artificial Sequence:  note = Synthetic Construct
ctggtaactgacccctcagc

SEQ ID NO: 135 - DNA, 20 bp, Artificial Sequence
  Description of Artificial Sequence:  note = Synthetic Construct
tgacaatgctttgggttttg

SEQ ID NO: 136 - DNA, 37 bp, Artificial Sequence
  Description of Artificial Sequence:  note = Synthetic Construct
ctttcacattccagacaatggagagtgtttatggttt

SEQ ID NO: 137 - DNA, 23 bp, Artificial Sequence
  Description of Artificial Sequence:  note = Synthetic Construct
tggcttcatatttcacatcaaga

SEQ ID NO: 138 - DNA, 20 bp, Artificial Sequence
  Description of Artificial Sequence:  note = Synthetic Construct
attctgccatcccaagtcat

SEQ ID NO: 139 - DNA, 52 bp, Artificial Sequence
  Description of Artificial Sequence:  note = Synthetic Construct
ccagttactgaaagcatcttaacccctcacatcccggctctggatggtactc

SEQ ID NO: 140 - DNA, 20 bp, Artificial Sequence
  Description of Artificial Sequence:  note = Synthetic Construct
ataaagggaattgggggaaa

SEQ ID NO: 141 - DNA, 23 bp, Artificial Sequence
  Description of Artificial Sequence:  note = Synthetic Construct
gaaaggctattacagggaagatg

SEQ ID NO: 142 - DNA, 60 bp, Artificial Sequence
  Description of Artificial Sequence:  note = Synthetic Construct
taattccaatattttcaatcatatttactattgtgatgcataaaaattcagctcagctac

SEQ ID NO: 143 - DNA, 17 bp, Artificial Sequence
  Description of Artificial Sequence:  note = Synthetic Construct
tgccgcgggggcagggg

SEQ ID NO: 144 - DNA, 15 bp, Artificial Sequence
  Description of Artificial Sequence:  note = Synthetic Construct
gcgggggcaggggca

SEQ ID NO: 145 - DNA, 17 bp, Artificial Sequence
  Description of Artificial Sequence:  note = Synthetic Construct
cctgcgcccgcggcatc

SEQ ID NO: 146 - DNA, 17 bp, Artificial Sequence
  Description of Artificial Sequence:  note = Synthetic Construct
atgccgcgggcgcaggg

SEQ ID NO: 147 - DNA, 17 bp, Artificial Sequence
  Description of Artificial Sequence:  note = Synthetic Construct
cctgcccctgcgcccgc

SEQ ID NO: 148 - DNA, 17 bp, Artificial Sequence
  Description of Artificial Sequence:  note = Synthetic Construct
cgggcgcaggggcaggc

SEQ ID NO: 149 - protein, 12 aa, Artificial Sequence
  Description of Artificial Sequence:  note = Synthetic Construct
  VARIANT (0)...(0): Xaa = any amino acid
LYSTHRXXXTRP

SEQ ID NO: 150 - protein, 12 aa, Artificial Sequence
  Description of Artificial Sequence:  note = Synthetic Construct
TYRTRPTHRASP

SEQ ID NO: 151 - protein, 11 aa, Artificial Sequence
  Description of Artificial Sequence:  note = Synthetic Construct
PRPRPRSERPR

SEQ ID NO: 152 - protein, 12 aa, Artificial Sequence
  Description of Artificial Sequence:  note = Synthetic Construct
ARGGLYGLYCYS

SEQ ID NO: 153 - protein, 27 aa, Artificial Sequence
  Description of Artificial Sequence:  note = Synthetic Construct
TYRPHETRPILEGLYTHRASPCYSASN

SEQ ID NO: 154 - DNA, 20 bp, Artificial Sequence
  Description of Artificial Sequence:  note = Synthetic Construct
cggatgactccatgtcaatg

SEQ ID NO: 155 - DNA, 20 bp, Artificial Sequence
  Description of Artificial Sequence:  note = Synthetic Construct
ggctatcaactgtgctgcaa

SEQ ID NO: 156 - DNA, 20 bp, Artificial Sequence
  Description of Artificial Sequence:  note = Synthetic Construct
cccttgactttgagcaggag

SEQ ID NO: 157 - DNA, 20 bp, Artificial Sequence
  Description of Artificial Sequence:  note = Synthetic Construct
acaggtccttacggatgtcg

SEQ ID NO: 158 - DNA, 2586 bp, Artificial Sequence
  Description of Artificial Sequence:  note = Synthetic Construct
cgcgcctctgatcgcgttccgggacacacaggcggcggctgcgggcgcagagcggagatgcagcggcttggggccaccctgctgtgcctgctgctggcggcggcggtccccacggcccccgcgcccgctccgacggcgacctcggctccagtcaagcccggcccggctctcagctacccgcaggaggaggccaccctcaatgagatgttccgcgaggttgaggaactgatggaggacacgcagcacaaattgcgcagcgcggtggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgagggagcctgcggctgccgccgctgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttcccctctggcttgacagcatgaggtgttgtgcatttgttcagctcccccaggctgttctccaggcttcacagtctggtgcttgggagagtcaggcagggttaaactgcaggagcagtttgccacccctgtccagattattggctgctttgcctctaccagttggcagacagccgtttgttctacatggctttgataattgtttgaggggaggagatggaaacaatgtggagtctccctctgattggttttggggaaatgtggagaagagtgccctgctttgcaaacatcaacctggcaaaaatgcaacaaatgaattttccacgcagttctttccatgggcataggtaagctgtgccttcagctgttgcagatgaaatgttctgttcaccctgcattacatgtgtttattcatccagcagtgttgctcagctcctacctctgtgccagggcagcattttcatatccaagatcaattccctctctcagcacagcctggggagggggtcattgttctcctcgtccatcagggatctcagaggctcagagactgcaagctgcttgcccaagtcacacagctagtgaagaccagagcagtttcatctggttgtgactctaagctcagtgctctctccactaccccacaccagccttggtgccaccaaaagtgctccccaaaaggaaggagaatgggatttttcttttgaggcatgcacatctggaattaaggtcaaactaattctcacatccctctaaaagtaaactactgttaggaacagcagtgttctcacagtgtggggcagccgtccttctaatgaagacaatgatattgacactgtccctctttggcagttgcattagtaactttgaaaggtatatgactgagcgtagcatacaggttaacctgcagaaacagtacttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaattatgtataaacataagttgcatttagaaatcaagcataaatcacttcaactgctcttctgtagttcttggatttcttttcccttttgactttgaataaatgtaaaatcctttcagccagaaaaagtaaaatagaaacaacctgtattaaaaatcttccataaaaaaaaaaaa

SEQ ID NO: 159 - DNA, 1053 bp, Artificial Sequence
  Description of Artificial Sequence:  note = Synthetic Construct
atgcagcggcttggggccaccctgctgtgcctgctgctggcggcggcggtccccacggcccccgcgcccgctccgacggcgacctcggctccagtcaagcccggcccggctctcagctacccgcaggaggaggccaccctcaatgagatgttccgcgaggttgaggaactgatggaggacacgcagcacaaattgcgcagcgcggtggaagagatggaggcagaagaagctgctgctaaagcatcatcagaagtgaacctggcaaacttacctcccagctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgagggagcctgcggctgccgccgctgcactgctgggaggggaagagatttag

SEQ ID NO: 160 - protein, 350 aa, Artificial Sequence
  Description of Artificial Sequence:  note = Synthetic Construct
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALGEPAAAAAALLGGEEI

SEQ ID NO: 161 - DNA, 6584 bp, Artificial Sequence
  Description of Artificial Sequence:  note = Synthetic Construct
tgctgccatgtgccgctgccacgggtacccagcctgtcgctaaactttccgggcgccagcccggctctgagtcgcgcttctcagcggagtgacccagggacggaggacccaggctggctggggactgtctgctcttctcggcgggatccgtggagagtcctttccctggaatccgagccctaaccgtctctccccagccctatccggcgaggagcggagcgctgccagcggaggcagcgccttcccgaagcagtttatctttggacggttttctttaaaggaaaaagcaaccaacaggttgccagccccggcgccacacacgagacgccggagggagaagccccggcccggattcctctgcctgtgtgcgtccctcgcgggctgctggaggcgaggggagggagggggcgatggctcggcctgacccatccgcgccgccctcgctgttgctgctgctcctagcgcagctggtgggccgggcggccgccgcgtccaaggccccggtgtgccaggaaatcacggtgcccatgtgccgcggcatcggctacaacctgacgcacatgcccaaccagttcaaccacgacacgcaggacgaggcgggcctggaggtgcaccagttctggccgctggtggagatccaatgctcgccggacctgcgcttcttcctatgctctatgtacacgcccatctgtctgcccgactaccacaagccgctgccgccctgccgctcggtgtgcgagcgcgccaaggccggctgctcgccgctgatgcgccagtacggcttcgcctggcccgagcgcatgagctgcgaccgcctcccggtgctgggccgcgacgccgaggtcctctgcatggattacaaccgcagcgaggccaccacggcgccccccaggcctttcccagccaagcccacccttccaggcccgccaggggcgccggcctcggggggcgaatgccccgctgggggcccgttcgtgtgcaagtgtcgcgagcccttcgtgcccattctgaaggagtcacacccgctctacaacaaggtgcggacgggccaggtgcccaactgcgcggtaccctgctaccagccgtccttcagtgccgacgagcgcacgttcgccaccttctggataggcctgtggtcggtgctgtgcttcatctccacgtccaccacagtggccaccttcctcatcgacatggaacgcttccgctatcctgagcgccccatcatcttcctgtcagcctgctacctgtgcgtgtcgctgggcttcctggtgcgtctggtcgtgggccatgccagcgtggcctgcagccgcgagcacaaccacatccactacgagaccacgggccctgcactgtgcaccatcgtcttcctcctggtctacttcttcggcatggccagctccatctggtgggtcatcctgtcgctcacctggttcctggccgccggcatgaagtggggcaacgaggccatcgcgggctacgcgcagtacttccacctggctgcgtggctcatccccagcgtcaagtccatcacggcactggcgctgagctccgtggacggggacccagtggccggcatctgctacgtgggcaaccagaacctgaactcgctgcgcggcttcgtgctgggcccgctggtgctctacctgctggtgggcacgctcttcctgctggcgggcttcgtgtcgctcttccgcatccgcagcgtcatcaagcagggcggcaccaagacggacaagctggagaagctcatgatccgcatcggcatcttcacgctgctctacacggtccccgccagcattgtggtggcctgctacctgtacgagcagcactaccgcgagagctgggaggcggcgctcacctgcgcctgcccgggccacgacaccggccagccgcgcgccaagcccgagtactgggtgctcatgctcaagtacttcatgtgcctggtggtgggcatcacgtcgggcgtctggatctggtcgggcaagacggtggagtcgtggcggcgtttcaccagccgctgctgctgccgcccgcggcgcggccacaagagcgggggcgccatggccgcaggggactaccccgaggcgagcgccgcgctcacaggcaggaccgggccgccgggccccgccgccacctaccacaagcaggtgtccctgtcgcacgtgtaggaggctgccgccgagggactcggccggagagctgaggggaggggggcgttttgtttggtagttttgccaaggtcacttccgtttaccttcatggtgctgttgccccctcccgcggcgacttggagagagggaagaggggcgttttcgaggaagaacctgtcccaggtcttctccaaggggcccagctcacgtgtattctattttgcgtttcttactgccttctttatgggaaccctctttttaatttatatgtatttttcttaatttgtaactttgttgcattttggcaacaatttacctttgctttgggggctttacaatcctaaggttggcgttgtaatgaagttccacttggttcaggtttctttgaactgtgtggtctcaattgggaaaatatatttcctatacgtgtgtctttaaaaaaaaatgtgaacagtgaacgtttcggttgctgtgactgggaagttgttgggtgtgctttttcagccagcttctccttccactgcttaaagtgtccatgattctttaaggtgagctgcagtttatagccccaggtcatacctaggaggggagcataatgagctcagggcctccccaaagtgacaaggttagggagtgcttagcggttttgtgttcagccttagctttgtttatagagggaggttcagtttcttttctgtagtgcttgtaataattctcactcctaacagcaccatcgttgtgtcttgaataagttagaggtagcattatagaggatctggcataaatatttgcagtagtgagagcctaagcgatggtgattggtggagcttgaattttaggctggtgagatggcagctttgtgcctgagaggtagtgggtggttcttaagcttcagtgatcccctttttttttttttttttttttttttaaggaacttgtgttataattttggtaaaagtataaacccactccctctggacaatacttagcgacagttgctaaagggggctcctttttaaatgtaaggactgaaatggatatacttctaataagtaaatttccaacacttatttgctccaccccctcccccctcccccctccccctttatcatgttaaacagcctttttgcttttcttattcctcctctcctggagagctgtgattagaaaccacacccacccttgaatgaagtgcttgaactgggggagggaggctggctacctgtgaacaaacattggcccaaataagggaaaataagtgttcctggactttggactagtttatagccagatattccaagagcagcaagacgttgctctctgccgtctctgaaaacaaaagagatgcataacatgcttgcacaaccttttaaaatatagatcagtatagtgctacctctatagttttcttcctcttctgagaaagcctgtatattgatgatcacacacacacacactttgcaattagagaatttggtttgctttactaatctgtttaactattccttcattcattatgaacgcttatattgatgaacatacacacagaggtttctttgctattagaaaattctgtttgctttcctaatctgtttaagcattcattcatgaagagtgtggggccattactggggaaggggggtgacagtgcctcagccagcaaaataccaatgaccaggattggggactaaatttaggaagctaaaatggccagagcaattaacatttgagaaaatcctgtctaggaaaacaacttgagtgtaggcatttgtaattcacttataccaaagttggaaaagtaaaatttaagcctaggacaatttttacttcatggatgttaaatagacaaatgcatagttcccagggggaatttaaacactttactggtgggaagaaacctagtattaaagttgtaaggactctcaaaaacttcacatttattaaaatgcactgctcttacccaatttatcctctgaattaaaatttcagtggattctacaaaacctcgtacaaatagctacagaactttgtgcctattttattcctctatttattcttctaggaagaagcctcttcctagaatcttgaaatagatcccttgactgaatgccaattcctctcctgtttttcaaatgagagaaccttttctgatcaccttgaccttttccctcatttcatatgtcttcccagaaagtagacagactgctctgctgccttcagtcattgtgcctcatttgggttgtccctccttctttgtggagaaatctggaaatgatgcacagtgtatccaaaagttgtgggatgaagtggatgaaagtgatttaattcatttttagaatttttttttgttttgttttagcaacatgctgaacaactaatttactttaaaaataagccagttaaaacaaaggacgctaagcccaagtggggggcaatattagtcaggatctttggggtctaattccagaccaactttcagaagcacttctttgtctctgttctcacctctgctgtccctctcttccctcatcccctaagagagacaaagataaaagcccacctgcatccctaagtcttactgagatcagccaccccaggggagagaaactggatctacttacagccaccccctgtttccatccatatacttacttcccccaatttgcatgtgattatggaaacaagtcatgctcatgaaagcaactgtaaaataaaaggttatggagtagttcagcaacttcttcacagccagctttgtggagctggggaggacttagggcccattggagtctcttatgtgtacagcttcagggctgtccctttcagtttgattttaagcaatgcctcacttcatagcttagggggtaaggattccattcaggtaggttgtctaaaggaactaatgggacctctcagtgaattagctgaccagattttaggaaatctttttaatttctatgattttccttctcacattttgaaatggtaaaattgactggaaataatttttcttggtgccttattggttttccttgcaaacctttctcatattttctcatgaccattgccagtgaccaaggcccatgtgtgtgttgtgtgtaattgtgggcatgtacaagcttaaataacgtgccgacagcactgtttcaaagttggtattcattaggctgttgcctcctgggctggagctgcgctaatcctgacaccggctgccaggagaaaacctcatggatcacacaccaaaccttaataacagcatccgtgacctgcactctccagtacagaatgggaaccccagagctaggaaatgtagttgtatattttaatgaactgctaccccagccaaagaagcttctttcacttttgtgctctacagaaagcccaaggggggtaggagggacagagctttgaataactgctttctaacactaaatgtggccaacaggacagagcacatcacacgtataggcaggtgtgagggacagtggctaagaattgcctgctccctctgcatgctctttcttgtttccaaagtccaatcaagtgatcctgggaaacaaatctgtctggattgcggagggtggttctgaaagaactgccaagacgttaaagaagggtgaagagtaggcagaatataagtagctaacctgagtcaagactctcaaaagctagcagcctgatgacaataggatttatttcagccaggatagtgtctgtctgtgagtgcatcattttaagacagtatgacttcatgttgttacaaactatgtatagtatgtatgttttgtgggttgtatatatacataatatatattatatatatatatgagagatttggtgacttttgatacgggtttggtgcaggtgaatttattactgagccaaatgaggcacataccgagtcagtagttgaagtccagggcattcgatactgtttatgatttccatatatgtatagtgcctatcccatgctgtagtcactgttatgttaaatccagaagttacactagagccagcgatactttatttgtagacaatcaatttgaatccatatgttattactggcagatgatacatgattacagttctgaatctgtaacacttacaaaaggaaacccagagcagcttgatgagtttttgtttctgcttcgttcctgggagtcagtagaaacagcagttgtatgtggttatgttagtctcaagatacttaatttgttgaccttacttcagaaaaattttgtatgtattatatttgtgggaaggtaaaataatcatttgagatttttatcaaatatgaagattagttatttatgaaaaacaaagaaatgtctatttttctttgttcccaattaatgtagataaattttaaaatgcattaaagtaatggtaaagacaataaaaagatgctgtagaaaaaaaaaaaaaaaaaaaaaa

SEQ ID NO: 162 - DNA, 1758 bp, Artificial Sequence
  Description of Artificial Sequence:  note = Synthetic Construct
atggctcggcctgacccatccgcgccgccctcgctgttgctgctgctcctagcgcagctggtgggccgggcggccgccgcgtccaaggccccggtgtgccaggaaatcacggtgcccatgtgccgcggcatcggctacaacctgacgcacatgcccaaccagttcaaccacgacacgcaggacgaggcgggcctggaggtgcaccagttctggccgctggtggagatccaatgctcgccggacctgcgcttcttcctatgctctatgtacacgcccatctgtctgcccgactaccacaagccgctgccgccctgccgctcggtgtgcgagcgcgccaaggccggctgctcgccgctgatgcgccagtacggcttcgcctggcccgagcgcatgagctgcgaccgcctcccggtgctgggccgcgacgccgaggtcctctgcatggattacaaccgcagcgaggccaccacggcgccccccaggcctttcccagccaagcccacccttccaggcccgccaggggcgccggcctcggggggcgaatgccccgctgggggcccgttcgtgtgcaagtgtcgcgagcccttcgtgcccattctgaaggagtcacacccgctctacaacaaggtgcggacgggccaggtgcccaactgcgcggtaccctgctaccagccgtccttcagtgccgacgagcgcacgttcgccaccttctggataggcctgtggtcggtgctgtgcttcatctccacgtccaccacagtggccaccttcctcatcgacatggaacgcttccgctatcctgagcgccccatcatcttcctgtcagcctgctacctgtgcgtgtcgctgggcttcctggtgcgtctggtcgtgggccatgccagcgtggcctgcagccgcgagcacaaccacatccactacgagaccacgggccctgcactgtgcaccatcgtcttcctcctggtctacttcttcggcatggccagctccatctggtgggtcatcctgtcgctcacctggttcctggccgccggcatgaagtggggcaacgaggccatcgcgggctacgcgcagtacttccacctggctgcgtggctcatccccagcgtcaagtccatcacggcactggcgctgagctccgtggacggggacccagtggccggcatctgctacgtgggcaaccagaacctgaactcgctgcgcggcttcgtgctgggcccgctggtgctctacctgctggtgggcacgctcttcctgctggcgggcttcgtgtcgctcttccgcatccgcagcgtcatcaagcagggcggcaccaagacggacaagctggagaagctcatgatccgcatcggcatcttcacgctgctctacacggtccccgccagcattgtggtggcctgctacctgtacgagcagcactaccgcgagagctgggaggcggcgctcacctgcgcctgcccgggccacgacaccggccagccgcgcgccaagcccgagtactgggtgctcatgctcaagtacttcatgtgcctggtggtgggcatcacgtcgggcgtctggatctggtcgggcaagacggtggagtcgtggcggcgtttcaccagccgctgctgctgccgcccgcggcgcggccacaagagcgggggcgccatggccgcaggggactaccccgaggcgagcgccgcgctcacaggcaggaccgggccgccgggccccgccgccacctaccacaagcaggtgtccctgtcgcacgtgtag

SEQ ID NO: 163 - protein, 585 aa, Artificial Sequence
  Description of Artificial Sequence:  note = Synthetic Construct
MARPDPSAPPSLLLLLLAQLVGRAAAASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFATFWIGLWSVLCFISTSTTVATFLIDMERFRYPERPIIFLSACYLCVSLGFLVRLVVGHASVACSREHNHIHYETTGPALCTIVFLLVYFFGMASSIWWVILSLTWFLAAGMKWGNEAIAGYAQYFHLAAWLIPSVKSITALALSSVDGDPVAGICYVGNQNLNSLRGFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHKSGGAMAAGDYPEASAALTGRTGPPGPAATYHKQVSLSHV

SEQ ID NO: 164 - DNA, 3195 bp, Artificial Sequence
  Description of Artificial Sequence:  note = Synthetic Construct
acagcatggagtggggttacctgttggaagtgacctcgctgctggccgccttggcgctgctgcagcgctctagcggcgctgcggccgcctcggccaaggagctggcatgccaagagatcaccgtgccgctgtgtaagggcatcggctacaactacacctacatgcccaatcagttcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccgccgcgcccagcccgccgcgccgcctgccgccgccgccgcccggcgagcagccgccttcgggcagcggccacggccgcccgccgggggccaggcccccgcaccgcggaggcggcaggggcggtggcggcggggacgcggcggcgcccccagctcgcggcggcggcggtggcgggaaggcgcggccccctggcggcggcgcggctccctgcgagcccgggtgccagtgccgcgcgcctatggtgagcgtgtccagcgagcgccacccgctctacaaccgcgtcaagacaggccagatcgctaactgcgcgctgccctgccacaacccctttttcagccaggacgagcgcgccttcaccgtcttctggatcggcctgtggtcggtgctctgcttcgtgtccaccttcgccaccgtctccaccttccttatcgacatggagcgcttcaagtacccggagcggcccattatcttcctctcggcctgctacctcttcgtgtcggtgggctacctagtgcgcctggtggcgggccacgagaaggtggcgtgcagcggtggcgcgccgggcgcggggggcgctgggggcgcgggcggcgcggcggcgggcgcgggcgcggcgggcgcgggcgcgggcggcccgggcgggcgcggcgagtacgaggagctgggcgcggtggagcagcacgtgcgctacgagaccaccggccccgcgctgtgcaccgtggtcttcttgctggtctacttcttcggcatggccagctccatctggtgggtgatcttgtcgctcacatggttcctggcggccggtatgaagtggggcaacgaagccatcgccggctactcgcagtacttccacctggccgcgtggcttgtgcccagcgtcaagtccatcgcggtgctggcgctcagctcggtggacggcgacccggtggcgggcatctgctacgtgggcaaccagagcctggacaacctgcgcggcttcgtgctggcgccgctggtcatctacctcttcatcggcaccatgttcctgctggccggcttcgtgtccctgttccgcatccgctcggtcatcaagcaacaggacggccccaccaagacgcacaagctggagaagctgatgatccgcctgggcctgttcaccgtgctctacaccgtgcccgccgcggtggtggtcgcctgcctcttctacgagcagcacaaccgcccgcgctgggaggccacgcacaactgcccgtgcctgcgggacctgcagcccgaccaggcacgcaggcccgactacgccgtcttcatgctcaagtacttcatgtgcctagtggtgggcatcacctcgggcgtgtgggtctggtccggcaagacgctggagtcctggcgctccctgtgcacccgctgctgctgggccagcaagggcgccgcggtgggcgggggcgcgggcgccacggccgcggggggtggcggcgggccggggggcggcggcggcgggggacccggcggcggcggggggccgggcggcggcgggggctccctctacagcgacgtcagcactggcctgacgtggcggtcgggcacggcgagctccgtgtcttatccaaagcagatgccattgtcccaggtctgagcggaggggagggggcgcccaggaggggtggggaggggggcgaggagacccaagtgcagcgaagggacacttgatgggctgaggttcccaccccttcacagtgttgattgctattagcatgataatgaactcttaatggtatccattagctgggacttaaatgactcacttagaacaaagtacctggcattgaagcctcccagacccagccccttttcctccattgatgtgcggggagctcctcccgccacgcgttaatttctgttggctgaggagggtggactctgcggcgtttccagaacccgagatttggagccctccctggctgcacttggctgggtttgcagtcagatacacagatttcacctgggagaacctctttttctccctcgactcttcctacgtaaactcccacccctgacttaccctggaggaggggtgaccgccacctgatgggattgcacggtttgggtattcttaatgaccaggcaaatgccttaagtaaacaaacaagaaatgtcttaattatacaccccacgtaaatacgggtttcttacattagaggatgtatttatataattatttgttaaattgtaaaaaaaaaaagtgtaaaatatgtatatatccaaagatatagtgtgtacatttttttgtaaaaagtttagaggcttacccctgtaagaacagatataagtattctattttgtcaataaaatgacttttgataaatgatttaaccattgccctctcccccgcctcttctgagctgtcacctttaaagtgcttgctaaggacgcatggggaaaatggacattttctggcttgtcattctgtacactgaccttaggcatggagaaaattacttgttaaactctagttcttaagttgttagccaagtaaatatcattgttgaactgaaatcaaaattgagtttttgcaccttccccaaagacggtgtttttcatgggagctcttttctgatccatggataacaactctcactttagtggatgtaaatggaacttctgcaaggcagtaattccccttaggccttgttatttatcctgcatggtatcactaaaggtttcaaaaccctgaaaaaaaa

SEQ ID NO: 165 - DNA, 2085 bp, Artificial Sequence
  Description of Artificial Sequence:  note = Synthetic Construct
atggagtggggttacctgttggaagtgacctcgctgctggccgccttggcgctgctgcagcgctctagcggcgctgcggccgcctcggccaaggagctggcatgccaagagatcaccgtgccgctgtgtaagggcatcggctacaactacacctacatgcccaatcagttcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccgccgcgcccagcccgccgcgccgcctgccgccgccgccgcccggcgagcagccgccttcgggcagcggccacggccgcccgccgggggccaggcccccgcaccgcggaggcggcaggggcggtggcggcggggacgcggcggcgcccccagctcgcggcggcggcggtggcgggaaggcgcggccccctggcggcggcgcggctccctgcgagcccgggtgccagtgccgcgcgcctatggtgagcgtgtccagcgagcgccacccgctctacaaccgcgtcaagacaggccagatcgctaactgcgcgctgccctgccacaacccctttttcagccaggacgagcgcgccttcaccgtcttctggatcggcctgtggtcggtgctctgcttcgtgtccaccttcgccaccgtctccaccttccttatcgacatggagcgcttcaagtacccggagcggcccattatcttcctctcggcctgctacctcttcgtgtcggtgggctacctagtgcgcctggtggcgggccacgagaaggtggcgtgcagcggtggcgcgccgggcgcggggggcgctgggggcgcgggcggcgcggcggcgggcgcgggcgcggcgggcgcgggcgcgggcggcccgggcgggcgcggcgagtacgaggagctgggcgcggtggagcagcacgtgcgctacgagaccaccggccccgcgctgtgcaccgtggtcttcttgctggtctacttcttcggcatggccagctccatctggtgggtgatcttgtcgctcacatggttcctggcggccggtatgaagtggggcaacgaagccatcgccggctactcgcagtacttccacctggccgcgtggcttgtgcccagcgtcaagtccatcgcggtgctggcgctcagctcggtggacggcgacccggtggcgggcatctgctacgtgggcaaccagagcctggacaacctgcgcggcttcgtgctggcgccgctggtcatctacctcttcatcggcaccatgttcctgctggccggcttcgtgtccctgttccgcatccgctcggtcatcaagcaacaggacggccccaccaagacgcacaagctggagaagctgatgatccgcctgggcctgttcaccgtgctctacaccgtgcccgccgcggtggtggtcgcctgcctcttctacgagcagcacaaccgcccgcgctgggaggccacgcacaactgcccgtgcctgcgggacctgcagcccgaccaggcacgcaggcccgactacgccgtcttcatgctcaagtacttcatgtgcctagtggtgggcatcacctcgggcgtgtgggtctggtccggcaagacgctggagtcctggcgctccctgtgcacccgctgctgctgggccagcaagggcgccgcggtgggcgggggcgcgggcgccacggccgcggggggtggcggcgggccggggggcggcggcggcgggggacccggcggcggcggggggccgggcggcggcgggggctccctctacagcgacgtcagcactggcctgacgtggcggtcgggcacggcgagctccgtgtcttatccaaagcagatgccattgtcccaggtctga

SEQ ID NO: 166 - protein, 694 aa, Artificial Sequence
  Description of Artificial Sequence:  note = Synthetic Construct
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 167 - DNA, 5161 bp, Artificial Sequence
  Description of Artificial Sequence:  note = Synthetic Construct
cgcgcgaggagccgccgccgccgcgccatggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggacttcaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacatttttgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtggacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgcgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgtacagtggagaaatatttataaacttaattttgtaaaacagaaaaaaaaaaaaaaaaaaaaaaaaaaaaaaaaaaa

SEQ ID NO: 168 - DNA, 5161 bp, Artificial Sequence; identical to SEQ ID NO: 167
  Description of Artificial Sequence:  note = Synthetic Construct

SEQ ID NO: 169 - protein, 1615 aa, Artificial Sequence
  Description of Artificial Sequence:  note = Synthetic Construct
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 170 - DNA, 5301 bp, Artificial Sequence
  Description of Artificial Sequence:  note = Synthetic Construct
gcggccgccccggctcctcgcctcccccacttctggccacccctcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacgtaaaaatgtaaatataaatttggttgagatctggagggggggagggagctattagagaaggatgaggcagaccatgtacagttaaaattataaaatggggtagggaatactggagatatttgtacagaagaaaaggatatttatatattttcttaaaacagcagatttgctgcttgtgccataaaagtttgtataaaaaaaatttgtactaaaagttttatttttgcaaactaaatacacaaagcatgccttaaacccagtgaagcaactgagtacaaaggaaacaggaataataaaggcatcactgaccaggaatatctgggctttattgataccaaaaaaaaaaaaa

SEQ ID NO: 171 - DNA, 4842 bp, Artificial Sequence
  Description of Artificial Sequence:  note = Synthetic Construct
atgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctga

SEQ ID NO: 172 - protein, 1613 aa, Artificial Sequence
  Description of Artificial Sequence:  note = Synthetic Construct
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAIVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 173 - DNA, 4350 bp, Artificial Sequence
  Description of Artificial Sequence:  note = Synthetic Construct
agttgagggattgacacaaatggtcaggcggcggcggcggagaaggaggcggaggcgcaggggggagccgagcccgctgggctgcggagagttgcgctctctacggggccgcggccactagcgcggcgccgccagccgggagccagcgagccgagggccaggaaggcgggacacgaccccggcgcgccctagccacccgggttctccccgccgcccgcgcttcatgaatcgcaagtttccgcggcggcggcggctgcggtacgcagaacaggagccgggggagcgggccgaaagcggcttgggctcgacggagggcacccgcgcagaggtctccctggccgcagggggagccgccgccggccgtgcccctggcagccccagcggagcggcgccaagagaggagccgagaaagtatggctgaggaggaggcgcctaagaagtcccgggccgccggcggtggcgcgagctgggaactttgtgccggggcgctctcggcccggctggcggaggagggcagcggggacgccggtggccgccgccgcccgccagttgacccccggcgattggcgcgccagctgctgctgctgctttggctgctggaggctccgctgctgctgggggtccgggcccaggcggcgggccaggggccaggccaggggcccgggccggggcagcaaccgccgccgccgcctcagcagcaacagagcgggcagcagtacaacggcgagcggggcatctccgtcccggaccacggctattgccagcccatctccatcccgctgtgcacggacatcgcgtacaaccagaccatcatgcccaacctgctgggccacacgaaccaggaggacgcgggcctggaggtgcaccagttctaccctctagtgaaagtgcagtgttccgctgagctcaagttcttcctgtgctccatgtacgcgcccgtgtgcaccgtgctagagcaggcgctgccgccctgccgctccctgtgcgagcgcgcgcgccagggctgcgaggcgctcatgaacaagttcggcttccagtggccagacacgctcaagtgtgagaagttcccggtgcacggcgccggcgagctgtgcgtgggccagaacacgtccgacaagggcaccccgacgccctcgctgcttccagagttctggaccagcaaccctcagcacggcggcggagggcaccgtggcggcttcccggggggcgccggcgcgtcggagcgaggcaagttctcctgcccgcgcgccctcaaggtgccctcctacctcaactaccacttcctgggggagaaggactgcggcgcaccttgtgagccgaccaaggtgtatgggctcatgtacttcgggcccgaggagctgcgcttctcgcgcacctggattggcatttggtcagtgctgtgctgcgcctccacgctcttcacggtgcttacgtacctggtggacatgcggcgcttcagctacccggagcggcccatcatcttcttgtccggctgttacacggccgtggccgtggcctacatcgccggcttcctcctggaagaccgagtggtgtgtaatgacaagttcgccgaggacggggcacgcactgtggcgcagggcaccaagaaggagggctgcaccatcctcttcatgatgctctacttcttcagcatggccagctccatctggtgggtgatcctgtcgctcacctggttcctggcggctggcatgaagtggggccacgaggccatcgaagccaactcacagtattttcacctggccgcctgggctgtgccggccatcaagaccatcaccatcctggcgctgggccaggtggacggcgatgtgctgagcggagtgtgcttcgtggggcttaacaacgtggacgcgctgcgtggcttcgtgctggcgcccctcttcgtgtacctgtttatcggcacgtcctttctgctggccggctttgtgtcgctcttccgcatccgcaccatcatgaagcacgatggcaccaagaccgagaagctggagaagctcatggtgcgcattggcgtcttcagcgtgctgtacactgtgccagccaccatcgtcatcgcctgctacttctacgagcaggccttccgggaccagtgggaacgcagctgggtggcccagagctgcaagagctacgctatcccctgccctcacctccaggcgggcggaggcgccccgccgcacccgcccatgagcccggacttcacggtcttcatgattaagtaccttatgacgctgatcgtgggcatcacgtcgggcttctggatctggtccggcaagaccctcaactcctggaggaagttctacacgaggctcaccaacagcaaacaaggggagactacagtctgagacccggggctcagcccatgcccaggcctcggccggggcgcagcgatcccccaaagccagcgccgtggagttcgtgccaatcctgacatctcgaggtttcctcactagacaactctctttcgcaggctcctttgaacaactcagctcctgcaaaagcttccgtccctgaggcaaaaggacacgagggcccgactgccagagggaggatggacagacctcttgccctcacactctggtaccaggactgttcgcttttatgattgtaaatagcctgtgtaagatttttgtaagtatatttgtatttaaatgacgaccgatcacgcgtttttctttttcaaaagtttttaattatttagggcggtttaaccatttgaggcttttccttcttgcccttttcggagtattgcaaaggagctaaaactggtgtgcaaccgcacagcgctcctggtcgtcctcgcgcgcctctccctaccacgggtgctcgggacggctgggcgccagctccggggcgagttcagcactgcggggtgcgactagggctgcgctgccagggtcacttcccgcctcctccttttgccccctccccctccttctgtcccctccctttctttcctggcttgaggtaggggctcttaaggtacagaactccacaaaccttccaaatctggaggagggcccccatacattacaattcctcccttgctcggcggtggattgcgaaggcccgtcccttcgacttcctgaagctggatttttaactgtccagaactttcctccaacttcatgggggcccacgggtgtgggcgctggcagtctcagcctccctccacggtcaccttcaacgcccagacactcccttctcccaccttagttggttacagggtgagtgagataaccaatgccaaactttttgaagtctaatttttgaggggtgagctcatttcattctctagtgtctaaaacctggtatgggtttggccagcgtcatggaaagatgtggttactgagatttgggaagaagcatgaagctttgtgtgggttggaagagactgaagatatgggttataaaatgttaattctaattgcatacggatgcctggcaaccttgcctttgagaatgagacagcctgcgcttagattttaccggtctgtaaaatggaaatgttgaggtcacctggaaagctttgttaaggagttgatgtttgctttccttaacaagacagcaaaacgtaaacagaaattgaaaacttgaaggatatttcagtgtcatggacttcctcaaaatgaagtgctattttcttatttttaatcaaataactagacatatatcagaaactttaaaatgtaaaagttgtacactttcaacattttattacgattattattcagcagcacattctgaggggggaacaattcacaccaccaataataacctggtaagatttcaggaggtaaagaaggtggaataattgacggggagatagcgcctgaaataaacaaaatatgggcatgcatgctaaagggaaaatgtgtgcaggtctactgcattaaatcctgtgtgctcctcttttggatttacagaaatgtgtcaaatgtaaatctttcaaagccatttaaaaatattcactttagttctctgtgaagaagaggagaaaagcaatcctcctgattgtattgttttaaactttaagaatttatcaaaatgccggtacttaggacctaaatttatctatgtctgtcatacgctaaaatgatattggtctttgaatttggtatacatttattctgttcactatcacaaaatcatctatatttatagaggaatagaagtttatatatatataataccatatttttaatttcacaaataaaaaattcaaagttttgtacaaaattatatggattttgtgcctgaaaataatagagcttgagctgtctgaactattttacattttatggtgtctcatagccaatcccacagtgtaaaaattca

SEQ ID NO: 174 - DNA, 1944 bp, Artificial Sequence
  Description of Artificial Sequence:  note = Synthetic Construct
atggctgaggaggaggcgcctaagaagtcccgggccgccggcggtggcgcgagctgggaactttgtgccggggcgctctcggcccggctggcggaggagggcagcggggacgccggtggccgccgccgcccgccagttgacccccggcgattggcgcgccagctgctgctgctgctttggctgctggaggctccgctgctgctgggggtccgggcccaggcggcgggccaggggccaggccaggggcccgggccggggcagcaaccgccgccgccgcctcagcagcaacagagcgggcagcagtacaacggcgagcggggcatctccgtcccggaccacggctattgccagcccatctccatcccgctgtgcacggacatcgcgtacaaccagaccatcatgcccaacctgctgggccacacgaaccaggaggacgcgggcctggaggtgcaccagttctaccctctagtgaaagtgcagtgttccgctgagctcaagttcttcctgtgctccatgtacgcgcccgtgtgcaccgtgctagagcaggcgctgccgccctgccgctccctgtgcgagcgcgcgcgccagggctgcgaggcgctcatgaacaagttcggcttccagtggccagacacgctcaagtgtgagaagttcccggtgcacggcgccggcgagctgtgcgtgggccagaacacgtccgacaagggcaccccgacgccctcgctgcttccagagttctggaccagcaaccctcagcacggcggcggagggcaccgtggcggcttcccggggggcgccggcgcgtcggagcgaggcaagttctcctgcccgcgcgccctcaaggtgccctcctacctcaactaccacttcctgggggagaaggactgcggcgcaccttgtgagccgaccaaggtgtatgggctcatgtacttcgggcccgaggagctgcgcttctcgcgcacctggattggcatttggtcagtgctgtgctgcgcctccacgctcttcacggtgcttacgtacctggtggacatgcggcgcttcagctacccggagcggcccatcatcttcttgtccggctgttacacggccgtggccgtggcctacatcgccggcttcctcctggaagaccgagtggtgtgtaatgacaagttcgccgaggacggggcacgcactgtggcgcagggcaccaagaaggagggctgcaccatcctcttcatgatgctctacttcttcagcatggccagctccatctggtgggtgatcctgtcgctcacctggttcctggcggctggcatgaagtggggccacgaggccatcgaagccaactcacagtattttcacctggccgcctgggctgtgccggccatcaagaccatcaccatcctggcgctgggccaggtggacggcgatgtgctgagcggagtgtgcttcgtggggcttaacaacgtggacgcgctgcgtggcttcgtgctggcgcccctcttcgtgtacctgtttatcggcacgtcctttctgctggccggctttgtgtcgctcttccgcatccgcaccatcatgaagcacgatggcaccaagaccgagaagctggagaagctcatggtgcgcattggcgtcttcagcgtgctgtacactgtgccagccaccatcgtcatcgcctgctacttctacgagcaggccttccgggaccagtgggaacgcagctgggtggcccagagctgcaagagctacgctatcccctgccctcacctccaggcgggcggaggcgccccgccgcacccgcccatgagcccggacttcacggtcttcatgattaagtaccttatgacgctgatcgtgggcatcacgtcgggcttctggatctggtccggcaagaccctcaactcctggaggaagttctacacgaggctcaccaacagcaaacaaggggagactacagtctga

SEQ ID NO: 175 - protein, 647 aa, Artificial Sequence
  Description of Artificial Sequence:  note = Synthetic Construct
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQAGGGAPPHPPMSPDFTVFMIKYLMTLIVGITSGFWIWSGKTLNSWRKFYTRLTNSKQGETTV

SEQ ID NO: 176 - DNA, 1983 bp, Artificial Sequence
  Description of Artificial Sequence:  note = Synthetic Construct
cgagtaaagtttgcaaagaggcgcgggaggcggcagccgcagcgaggaggcggcggggaagaagcgcagtctccgggttgggggcgggggcggggggggcgccaaggagccgggtggggggcggcggccagcatgcggccccgcagcgccctgccccgcctgctgctgccgctgctgctgctgcccgccgccgggccggcccagttccacggggagaagggcatctccatcccggaccacggcttctgccagcccatctccatcccgctgtgcacggacatcgcctacaaccagaccatcatgcccaaccttctgggccacacgaaccaggaggacgcaggcctagaggtgcaccagttctatccgctggtgaaggtgcagtgctcgcccgaactgcgcttcttcctgtgctccatgtacgcacccgtgtgcaccgtgctggaacaggccatcccgccgtgccgctctatctgtgagcgcgcgcgccagggctgcgaagccctcatgaacaagttcggttttcagtggcccgagcgcctgcgctgcgagcacttcccgcgccacggcgccgagcagatctgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgcccgcgcgtcctcaaggtgccatcctatctcagctacaagtttctgggcgagcgtgattgtgctgcgccctgcgaacctgcgcggcccgatggttccatgttcttctcacaggaggagacgcgtttcgcgcgcctctggatcctcacctggtcggtgctgtgctgcgcttccaccttcttcactgtcaccacgtacttggtagacatgcagcgcttccgctacccagagcggcctatcatttttctgtcgggctgctacaccatggtgtcggtggcctacatcgcgggcttcgtgctccaggagcgcgtggtgtgcaacgagcgcttctccgaggacggttaccgcacggtggtgcagggcaccaagaaggagggctgcaccatcctcttcatgatgctctacttcttcagcatggccagctccatctggtgggtcatcctgtcgctcacctggttcctggcagccggcatgaagtggggccacgaggccatcgaggccaactctcagtacttccacctggccgcctgggccgtgccggccgtcaagaccatcaccatcctggccatgggccagatcgacggcgacctgctgagcggcgtgtgcttcgtaggcctcaacagcctggacccgctgcggggcttcgtgctagcgccgctcttcgtgtacctgttcatcggcacgtccttcctcctggccggcttcgtgtcgctcttccgcatccgcaccatcatgaagcacgacggcaccaagaccgaaaagctggagcggctcatggtgcgcatcggcgtcttctccgtgctctacacagtgcccgccaccatcgtcatcgcttgctacttctacgagcaggccttccgcgagcactgggagcgctcgtgggtgagccagcactgcaagagcctggccatcccgtgcccggcgcactacacgccgcgcatgtcgcccgacttcacggtctacatgatcaaatacctcatgacgctcatcgtgggcatcacgtcgggcttctggatctggtcgggcaagacgctgcactcgtggaggaagttctacactcgcctcaccaacagccgacacggtgagaccaccgtgtgagggacgcccccaggccggaaccgcgcggcgctttcctccgcccggggtggggcccctacagactccgtattttatttttttaaataaaaaacgatcgaaaccatttcacttttaggttgctttttaaaagagaactctctgcccaacaccccc

SEQ ID NO: 177 - DNA, 1698 bp, Artificial Sequence
  Description of Artificial Sequence:  note = Synthetic Construct
atgcggccccgcagcgccctgccccgcctgctgctgccgctgctgctgctgcccgccgccgggccggcccagttccacggggagaagggcatctccatcccggaccacggcttctgccagcccatctccatcccgctgtgcacggacatcgcctacaaccagaccatcatgcccaaccttctgggccacacgaaccaggaggacgcaggcctagaggtgcaccagttctatccgctggtgaaggtgcagtgctcgcccgaactgcgcttcttcctgtgctccatgtacgcacccgtgtgcaccgtgctggaacaggccatcccgccgtgccgctctatctgtgagcgcgcgcgccagggctgcgaagccctcatgaacaagttcggttttcagtggcccgagcgcctgcgctgcgagcacttcccgcgccacggcgccgagcagatctgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgcccgcgcgtcctcaaggtgccatcctatctcagctacaagtttctgggcgagcgtgattgtgctgcgccctgcgaacctgcgcggcccgatggttccatgttcttctcacaggaggagacgcgtttcgcgcgcctctggatcctcacctggtcggtgctgtgctgcgcttccaccttcttcactgtcaccacgtacttggtagacatgcagcgcttccgctacccagagcggcctatcatttttctgtcgggctgctacaccatggtgtcggtggcctacatcgcgggcttcgtgctccaggagcgcgtggtgtgcaacgagcgcttctccgaggacggttaccgcacggtggtgcagggcaccaagaaggagggctgcaccatcctcttcatgatgctctacttcttcagcatggccagctccatctggtgggtcatcctgtcgctcacctggttcctggcagccggcatgaagtggggccacgaggccatcgaggccaactctcagtacttccacctggccgcctgggccgtgccggccgtcaagaccatcaccatcctggccatgggccagatcgacggcgacctgctgagcggcgtgtgcttcgtaggcctcaacagcctggacccgctgcggggcttcgtgctagcgccgctcttcgtgtacctgttcatcggcacgtccttcctcctggccggcttcgtgtcgctcttccgcatccgcaccatcatgaagcacgacggcaccaagaccgaaaagctggagcggctcatggtgcgcatcggcgtcttctccgtgctctacacagtgcccgccaccatcgtcatcgcttgctacttctacgagcaggccttccgcgagcactgggagcgctcgtgggtgagccagcactgcaagagcctggccatcccgtgcccggcgcactacacgccgcgcatgtcgcccgacttcacggtctacatgatcaaatacctcatgacgctcatcgtgggcatcacgtcgggcttctggatctggtcgggcaagacgctgcactcgtggaggaagttctacactcgcctcaccaacagccgacacggtgagaccaccgtgtga

SEQ ID NO: 178 - protein, 565 aa, Artificial Sequence
  Description of Artificial Sequence:  note = Synthetic Construct
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 179 - DNA, 3933 bp, Artificial Sequence
  Description of Artificial Sequence:  note = Synthetic Construct
gccgctccgggtacctgagggacgcgcggccgcccgcggcaggcggtgcagcccccccaccccttggagccaggcgccggggtctgaggatagcatttctcaagacctgacttatggagcacttgtaacctgagatatttcagttgaaggaagaaatagctcttctcctaagatggaatctgtggtttgggaatgtggttgatcaacttgatatgttggccaaatgtgccccatgtaataaaatgaaaagaagagacaagatgatgtcattttcccatattgtgaaaccaaaaacaaacgccttttgtgagaccaagctaacaaacctctgacggtgcgaagagtatttaactgtttgaagaatttaacagtaagatacagaagaagtaccttcgagctgagacctgcaggtgtataaatatctaaaatacatattgaataggcctgatcatctgaatctccttcagacccaggaaggatggctatgacttggattgtcttctctctttggcccttgactgtgttcatggggcatataggtgggcacagtttgttttcttgtgaacctattaccttgaggatgtgccaagatttgccttataatactaccttcatgcctaatcttctgaatcattatgaccaacagacagcagctttggcaatggagccattccaccctatggtgaatctggattgttctcgggatttccggccttttctttgtgcactctacgctcctatttgtatggaatatggacgtgtcacacttccctgtcgtaggctgtgtcagcgggcttacagtgagtgttcgaagctcatggagatgtttggtgttccttggcctgaagatatggaatgcagtaggttcccagattgtgatgagccatatcctcgacttgtggatctgaatttagctggagaaccaactgaaggagccccagtggcagtgcagagagactatggtttttggtgtccccgagagttaaaaattgatcctgatctgggttattcttttctgcatgtgcgtgattgttcacctccttgtccaaatatgtacttcagaagagaagaactgtcatttgctcgctatttcataggattgatttcaatcatttgcctctcggccacattgtttacttttttaacttttttgattgatgtcacaagattccgttatcctgaaaggcctattatattttatgcagtctgctacatgatggtatccttaattttcttcattggatttttgcttgaagatcgagtagcctgcaatgcatccatccctgcacaatataaggcttccacagtgacacaaggatctcataataaagcctgtaccatgctttttatgatactctatttttttactatggctggcagtgtatggtgggtaattcttaccatcacatggtttttagcagctgtgccaaagtggggtagtgaagctattgagaagaaagcattgctgtttcacgccagtgcatggggcatccccggaactctaaccatcatccttttagcgatgaataaaattgaaggtgacaatattagtggcgtgtgttttgttggcctctacgatgttgatgcattgagatattttgttcttgctcccctctgcctgtatgtggtagttggggtttctctcctcttagctggcattatatccctaaacagagttcgaattgagattccattagaaaaggagaaccaagataaattagtgaagtttatgatccggatcggtgttttcagcattctttatctcgtaccactcttggttgtaattggatgctacttttatgagcaagcttaccggggcatctgggaaacaacgtggatacaagaacgctgcagagaatatcacattccatgtccatatcaggttactcaaatgagtcgtccagacttgattctctttctgatgaaatacctgatggctctcatagttggcattccctctgtattttgggttggaagcaaaaagacatgctttgaatgggccagtttttttcatggtcgtaggaaaaaagagatagtgaatgagagccgacaggtactccaggaacctgattttgctcagtctctcctgagggatccaaatactcctatcataagaaagtcaaggggaacttccactcaaggaacatccacccatgcttcttcaactcagctggctatggtggatgatcaaagaagcaaagcaggaagcatccacagcaaagtgagcagctaccacggcagcctccacagatcacgtgatggcaggtacacgccctgcagttacagaggaatggaggagagactacctcatggcagcatgtcacgactaacagatcactccaggcatagtagttctcatcggctcaatgaacagtcacgacatagcagcatcagagatctcagtaataatcccatgactcatatcacacatggcaccagcatgaatcgggttattgaagaagatggaaccagtgcttaatttgtcttgtctaaggtggaaatcttgtgctgtttaaaaagcagattttattctttgccttttgcatgactgatagctgtaactcacagttaacatgctttcagtcaagtacagattgtgtccactggaaaggtaaatgattgcttttttatattgcatcaaacttggaacatcaaggcatccaaaacactaagaattctatcatcacaaaaataattcgtctttctaggttatgaagagataattatttgtctggtaagcatttttataaacccactcattttatatttagaaaaatcctaaatgtgtggtgactgctttgtagtgaactttcatatactataaactagttgtgagataacattctggtagctcagttaataaaacaatttcagaattaaagaaattttctatgcaaggtttacttctcagatgaacagtaggactttgtagttttatttccactaagtgaaaaaagaactgtgtttttaaactgtaggagaatttaataaatcagcaagggtattttagctaatagaataaaagtgcaacagaagaatttgattagtctatgaaaggttctcttaaaattctatcgaaataatcttcatgcagagatattcagggtttggattagcagtggaataaagagatgggcattgtttcccctataattgtgctgtttttataacttttgtaaatattactttttctggctgtgtttttataacttatccatatgcatgatggaaaaattttaatttgtagccatcttttcccatgtaatagtattgattcatagagaacttaatgttcaaaatttgctttgtggaggcatgtaataagataaacatcatacattataaggtaaccacaattacaaaatggcaaaacattttctctgtattcattgttgtatttttctacagtgagatgtgatcttgccaaagccaccagaccttggcttccaggccctcctgtagtgagttgattgtctgcacttgccttgcccaatagccagtaggctacagcttttgccccacacccttattttcagattctggatcattcttgtttacaactgaaatatatataacctcagtccaaagtggtgattgatttgagtatttgaaaattgttgtagctaaatgaagcatgattagtcttagtatgaatatcatttaatctttaaaaaatcaagtaaaaatgtttatctgataatgtttaaataatttacaatataaactgtaaaacttattaggcatgaaatcaatcagaagagaaagaaaaatgctggaacatgcttgatgtattatgtaaaaagcatatttaaacaagggtcctcaaccctgactgcagataagaatcacttgggttacttcagatgcctaacaccttcctctcatacaaataagaattggtagctttcttaaaaaaaaaaaaaaaaaaaaaaa

SEQ ID NO: 180 - DNA, 2001 bp, Artificial Sequence
  Description of Artificial Sequence:  note = Synthetic Construct
atggctatgacttggattgtcttctctctttggcccttgactgtgttcatggggcatataggtgggcacagtttgttttcttgtgaacctattaccttgaggatgtgccaagatttgccttataatactaccttcatgcctaatcttctgaatcattatgaccaacagacagcagctttggcaatggagccattccaccctatggtgaatctggattgttctcgggatttccggccttttctttgtgcactctacgctcctatttgtatggaatatggacgtgtcacacttccctgtcgtaggctgtgtcagcgggcttacagtgagtgttcgaagctcatggagatgtttggtgttccttggcctgaagatatggaatgcagtaggttcccagattgtgatgagccatatcctcgacttgtggatctgaatttagctggagaaccaactgaaggagccccagtggcagtgcagagagactatggtttttggtgtccccgagagttaaaaattgatcctgatctgggttattcttttctgcatgtgcgtgattgttcacctccttgtccaaatatgtacttcagaagagaagaactgtcatttgctcgctatttcataggattgatttcaatcatttgcctctcggccacattgtttacttttttaacttttttgattgatgtcacaagattccgttatcctgaaaggcctattatattttatgcagtctgctacatgatggtatccttaattttcttcattggatttttgcttgaagatcgagtagcctgcaatgcatccatccctgcacaatataaggcttccacagtgacacaaggatctcataataaagcctgtaccatgctttttatgatactctatttttttactatggctggcagtgtatggtgggtaattcttaccatcacatggtttttagcagctgtgccaaagtggggtagtgaagctattgagaagaaagcattgctgtttcacgccagtgcatggggcatccccggaactctaaccatcatccttttagcgatgaataaaattgaaggtgacaatattagtggcgtgtgttttgttggcctctacgatgttgatgcattgagatattttgttcttgctcccctctgcctgtatgtggtagttggggtttctctcctcttagctggcattatatccctaaacagagttcgaattgagattccattagaaaaggagaaccaagataaattagtgaagtttatgatccggatcggtgttttcagcattctttatctcgtaccactcttggttgtaattggatgctacttttatgagcaagcttaccggggcatctgggaaacaacgtggatacaagaacgctgcagagaatatcacattccatgtccatatcaggttactcaaatgagtcgtccagacttgattctctttctgatgaaatacctgatggctctcatagttggcattccctctgtattttgggttggaagcaaaaagacatgctttgaatgggccagtttttttcatggtcgtaggaaaaaagagatagtgaatgagagccgacaggtactccaggaacctgattttgctcagtctctcctgagggatccaaatactcctatcataagaaagtcaaggggaacttccactcaaggaacatccacccatgcttcttcaactcagctggctatggtggatgatcaaagaagcaaagcaggaagcatccacagcaaagtgagcagctaccacggcagcctccacagatcacgtgatggcaggtacacgccctgcagttacagaggaatggaggagagactacctcatggcagcatgtcacgactaacagatcactccaggcatagtagttctcatcggctcaatgaacagtcacgacatagcagcatcagagatctcagtaataatcccatgactcatatcacacatggcaccagcatgaatcgggttattgaagaagatggaaccagtgcttaa

SEQ ID NO: 181 - protein, 666 aa, Artificial Sequence
  Description of Artificial Sequence:  note = Synthetic Construct
MAMTWIVFSLWPLTVFMGHIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLAGEPTEGAPVAVQRDYGFWCPRELKIDPDLGYSFLHVRDCSPPCPNMYFRREELSFARYFIGLISIICLSATLFTFLTFLIDVTRFRYPERPIIFYAVCYMMVSLIFFIGFLLEDRVACNASIPAQYKASTVTQGSHNKACTMLFMILYFFTMAGSVWWVILTITWFLAAVPKWGSEAIEKKALLFHASAWGIPGTLTIILLAMNKIEGDNISGVCFVGLYDVDALRYFVLAPLCLYVVVGVSLLLAGIISLNRVRIEIPLEKENQDKLVKFMIRIGVFSILYLVPLLVVIGCYFYEQAYRGIWETTWIQERCREYHIPCPYQVTQMSRPDLILFLMKYLMALIVGIPSVFWVGSKKTCFEWASFFHGRRKKEIVNESRQVLQEPDFAQSLLRDPNTPIIRKSRGTSTQGTSTHASSTQLAMVDDQRSKAGSIHSKVSSYHGSLHRSRDGRYTPCSYRGMEERLPHGSMSRLTDHSRHSSSHRLNEQSRHSSIRDLSNNPMTHITHGTSMNRVIEEDGTSA

SEQ ID NO: 182 - DNA, 3851 bp, Artificial Sequence
  Description of Artificial Sequence:  note = Synthetic Construct
ctctcccaaccgcctcgtcgcactcctcaggctgagagcaccgctgcactcgcggccggcgatgcgggaccccggcgcggccgctccgctttcgtccctgggcctctgtgccctggtgctggcgctgctgggcgcactgtccgcgggcgccggggcgcagccgtaccacggagagaagggcatctccgtgccggaccacggcttctgccagcccatctccatcccgctgtgcacggacatcgcctacaaccagaccatcctgcccaacctgctgggccacacgaaccaagaggacgcgggcctcgaggtgcaccagttctacccgctggtgaaggtgcagtgttctcccgaactccgctttttcttatgctccatgtatgcgcccgtgtgcaccgtgctcgatcaggccatcccgccgtgtcgttctctgtgcgagcgcgcccgccagggctgcgaggcgctcatgaacaagttcggcttccagtggcccgagcggctgcgctgcgagaacttcccggtgcacggtgcgggcgagatctgcgtgggccagaacacgtcggacggctccgggggcccaggcggcggccccactgcctaccctaccgcgccctacctgccggacctgcccttcaccgcgctgcccccgggggcctcagatggcagggggcgtcccgccttccccttctcatgcccccgtcagctcaaggtgcccccgtacctgggctaccgcttcctgggtgagcgcgattgtggcgccccgtgcgaaccgggccgtgccaacggcctgatgtactttaaggaggaggagaggcgcttcgcccgcctctgggtgggcgtgtggtccgtgctgtgctgcgcctcgacgctctttaccgttctcacctacctggtggacatgcggcgcttcagctacccagagcggcccatcatcttcctgtcgggctgctacttcatggtggccgtggcgcacgtggccggcttccttctagaggaccgcgccgtgtgcgtggagcgcttctcggacgatggctaccgcacggtggcgcagggcaccaagaaggagggctgcaccatcctcttcatggtgctctacttcttcggcatggccagctccatctggtgggtcattctgtctctcacttggttcctggcggccggcatgaagtggggccacgaggccatcgaggccaactcgcagtacttccacctggccgcgtgggccgtgcccgccgtcaagaccatcactatcctggccatgggccaggtagacggggacctgctgagcggggtgtgctacgttggcctctccagtgtggacgcgctgcggggcttcgtgctggcgcctctgttcgtctacctcttcataggcacgtccttcttgctggccggcttcgtgtccctcttccgtatccgcaccatcatgaaacacgacggcaccaagaccgagaagctggagaagctcatggtgcgcatcggcgtcttcagcgtgctctacacagtgcccgccaccatcgtcctggcctgctacttctacgagcaggccttccgcgagcactgggagcgcacctggctcctgcagacgtgcaagagctatgccgtgccctgcccgcccggccacttcccgcccatgagccccgacttcaccgtcttcatgatcaagtacctgatgaccatgatcgtcggcatcaccactggcttctggatctggtcgggcaagaccctgcagtcgtggcgccgcttctaccacagacttagccacagcagcaagggggagactgcggtatgagccccggcccctccccacctttcccaccccagccctcttgcaagaggagaggcacggtagggaaaagaactgctgggtgggggcctgtttctgtaactttctccccctctactgagaagtgacctggaagtgagaagttctttgcagatttggggcgaggggtgatttggaaaagaagacctgggtggaaagcggtttggatgaaaagatttcaggcaaagacttgcaggaagatgatgataacggcgatgtgaatcgtcaaaggtacgggccagcttgtgcctaatagaaggttgagaccagcagagactgctgtgagtttctcccggctccgaggctgaacggggactgtgagcgatccccctgctgcagggcgagtggcctgtccagacccctgtgaggccccgggaaaggtacagccctgtctgcggtggctgctttgttggaaagagggagggcctcctgcggtgtgcttgtcaagcagtggtcaaaccataatctcttttcactggggccaaactggagcccagatgggttaatttccagggtcagacattacggtctctcctcccctgccccctcccgcctgtttttcctcccgtactgctttcaggtcttgtaaaataagcatttggaagtcttgggaggcctgcctgctagaatcctaatgtgaggatgcaaaagaaatgatgataacattttgagataaggccaaggagacgtggagtaggtatttttgctactttttcattttctggggaaggcaggaggcagaaagacgggtgttttatttggtctaataccctgaaaagaagtgatgacttgttgcttttcaaaacaggaatgcatttttccccttgtctttgttgtaagagacaaaagaggaaacaaaagtgtctccctgtggaaaggcataactgtgacgaaagcaacttttataggcaaagcagcgcaaatctgaggtttcccgttggttgttaatttggttgagataaacattcctttttaaggaaaagtgaagagcagtgtgctgtcacacaccgttaagccagaggttctgacttcgctaaaggaaatgtaagaggttttgttgtctgttttaaataaatttaattcggaacacatgatccaacagactatgttaaaatattcagggaaatctctcccttcatttactttttcttgctataagcctatatttaggtttcttttctatttttttctcccatttggatcctttgaggtaaaaaaacataatgtcttcagcctcataataaaggaaagttaattaaaaaaaaaaagcaaagagccattttgtcctgttttcttggttccatcaatctgtttattaaacatcatccatatgctgaccctgtctctgtgtggttgggttgggaggcgatcagcagataccatagtgaacgaagaggaaggtttgaaccatgggccccatctttaaagaaagtcattaaaagaaggtaaacttcaaagtgattctggagttctttgaaatgtgctggaagacttaaatttattaatcttaaatcatgtactttttttctgtaatagaactcggattcttttgcatgatggggtaaagcttagcagagaatcatgggagctaacctttatcccacctttgacactaccctccaatcttgcaacactatcctgtttctcagaacagtttttaaatgccaatcatagagggtactgtaaagtgtacaagttactttatatatgtaatgttcacttgagtggaactgctttttacattaaagttaaaatcgatcttgtgtttcttcaaccttcaaaactatctcatctgtcagatttttaaaactccaacacaggttttggcatcttttgtgctgtatcttttaagtgcatgtgaaatttgtaaaatagagataagtacagtatgtatattttgtaaatctcccatttttgtaagaaaatatatattgtatttatacatttttactttggatttttgttttgttggctttaaaggtctaccccactttatcacatgtacagatcacaaataaatttttttaaatac

SEQ ID NO: 183 - DNA, 1725 bp, Artificial Sequence
  Description of Artificial Sequence:  note = Synthetic Construct
atgcgggaccccggcgcggccgctccgctttcgtccctgggcctctgtgccctggtgctggcgctgctgggcgcactgtccgcgggcgccggggcgcagccgtaccacggagagaagggcatctccgtgccggaccacggcttctgccagcccatctccatcccgctgtgcacggacatcgcctacaaccagaccatcctgcccaacctgctgggccacacgaaccaagaggacgcgggcctcgaggtgcaccagttctacccgctggtgaaggtgcagtgttctcccgaactccgctttttcttatgctccatgtatgcgcccgtgtgcaccgtgctcgatcaggccatcccgccgtgtcgttctctgtgcgagcgcgcccgccagggctgcgaggcgctcatgaacaagttcggcttccagtggcccgagcggctgcgctgcgagaacttcccggtgcacggtgcgggcgagatctgcgtgggccagaacacgtcggacggctccgggggcccaggcggcggccccactgcctaccctaccgcgccctacctgccggacctgcccttcaccgcgctgcccccgggggcctcagatggcagggggcgtcccgccttccccttctcatgcccccgtcagctcaaggtgcccccgtacctgggctaccgcttcctgggtgagcgcgattgtggcgccccgtgcgaaccgggccgtgccaacggcctgatgtactttaaggaggaggagaggcgcttcgcccgcctctgggtgggcgtgtggtccgtgctgtgctgcgcctcgacgctctttaccgttctcacctacctggtggacatgcggcgcttcagctacccagagcggcccatcatcttcctgtcgggctgctacttcatggtggccgtggcgcacgtggccggcttccttctagaggaccgcgccgtgtgcgtggagcgcttctcggacgatggctaccgcacggtggcgcagggcaccaagaaggagggctgcaccatcctcttcatggtgctctacttcttcggcatggccagctccatctggtgggtcattctgtctctcacttggttcctggcggccggcatgaagtggggccacgaggccatcgaggccaactcgcagtacttccacctggccgcgtgggccgtgcccgccgtcaagaccatcactatcctggccatgggccaggtagacggggacctgctgagcggggtgtgctacgttggcctctccagtgtggacgcgctgcggggcttcgtgctggcgcctctgttcgtctacctcttcataggcacgtccttcttgctggccggcttcgtgtccctcttccgtatccgcaccatcatgaaacacgacggcaccaagaccgagaagctggagaagctcatggtgcgcatcggcgtcttcagcgtgctctacacagtgcccgccaccatcgtcctggcctgctacttctacgagcaggccttccgcgagcactgggagcgcacctggctcctgcagacgtgcaagagctatgccgtgccctgcccgcccggccacttcccgcccatgagccccgacttcaccgtcttcatgatcaagtacctgatgaccatgatcgtcggcatcaccactggcttctggatctggtcgggcaagaccctgcagtcgtggcgccgcttctaccacagacttagccacagcagcaagggggagactgcggtatga

SEQ ID NO: 184 - protein, 574 aa, Artificial Sequence
  Description of Artificial Sequence:  note = Synthetic Construct
MRDPGAAAPLSSLGLCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGRGRPAFPFSCPRQLKVPPYLGYRFLGERDCGAPCEPGRANGLMYFKEEERRFARLWVGVWSVLCCASTLFTVLTYLVDMRRFSYPERPIIFLSGCYFMVAVAHVAGFLLEDRAVCVERFSDDGYRTVAQGTKKEGCTILFMVLYFFGMASSIWWVILSLTWFLAAGMKWGHEAIEANSQYFHLAAWAVPAVKTITILAMGQVDGDLLSGVCYVGLSSVDALRGFVLAPLFVYLFIGTSFLLAGFVSLFRIRTIMKHDGTKTEKLEKLMVRIGVFSVLYTVPATIVLACYFYEQAFREHWERTWLLQTCKSYAVPCPPGHFPPMSPDFTVFMIKYLMTMIVGITTGFWIWSGKTLQSWRRFYHRLSHSSKGETAV

SEQ ID NO: 185 - DNA, 3260 bp, Artificial Sequence
  Description of Artificial Sequence:  note = Synthetic Construct
tcgaaacagctgccggctggtcccggccgaggccggcgcagggagggaggagccgcccgggctgtgggggcgccgcgagctgggccggcctcggtgtgcccgcgccgccagcccgctccagacgcgccacctgggcgctccaagaagaggccgaagtttgccgcggccgtgagttggagctcgcgccgggccgctgcgccgggagctccgggggcttccctcgcttcccggtattgtttgcaaactttgctgctctccgccgcggcccccaactcggcggacgccgggcgcggagagccgagccgggggcgctgtgcgcagcgctcgggccaggccgggcgggcatgggcgggggcccgagcaggggtggagagccggggccagcagcagcccgtgcccgggagcggcggcgctgaggggcgcggagctccccgcgaggacacgtccaacgccagcatgcagcgcccgggcccccgcctgtggctggtcctgcaggtgatgggctcgtgcgccgccatcagctccatggacatggagcgcccgggcgacggcaaatgccagcccatcgagatcccgatgtgcaaggacatcggctacaacatgactcgtatgcccaacctgatgggccacgagaaccagcgcgaggcagccatccagttgcacgagttcgcgccgctggtggagtacggctgccacggccacctccgcttcttcctgtgctcgctgtacgcgccgatgtgcaccgagcaggtctctacccccatccccgcctgccgggtcatgtgcgagcaggcccggctcaagtgctccccgattatggagcagttcaacttcaagtggcccgactccctggactgccggaaactccccaacaagaacgaccccaactacctgtgcatggaggcgcccaacaacggctcggacgagcccacccggggctcgggcctgttcccgccgctgttccggccgcagcggccccacagcgcgcaggagcacccgctgaaggacgggggccccgggcgcggcggctgcgacaacccgggcaagttccaccacgtggagaagagcgcgtcgtgcgcgccgctctgcacgcccggcgtggacgtgtactggagccgcgaggacaagcgcttcgcagtggtctggctggccatctgggcggtgctgtgcttcttctccagcgccttcaccgtgctcaccttcctcatcgacccggcccgcttccgctaccccgagcgccccatcatcttcctctccatgtgctactgcgtctactccgtgggctacctcatccgcctcttcgccggcgccgagagcatcgcctgcgaccgggacagcggccagctctatgtcatccaggagggactggagagcaccggctgcacgctggtcttcctggtcctctactacttcggcatggccagctcgctgtggtgggtggtcctcacgctcacctggttcctggccgccggcaagaagtggggccacgaggccatcgaagccaacagcagctacttccacctggcagcctgggccatcccggcggtgaagaccatcctgatcctggtcatgcgcagggtggcgggggacgagctcaccggggtctgctacgtgggcagcatggacgtcaacgcgctcaccggcttcgtgctcattcccctggcctgctacctggtcatcggcacgtccttcatcctctcgggcttcgtggccctgttccacatccggagggtgatgaagacgggcggcgagaacacggacaagctggagaagctcatggtgcgtatcgggctcttctctgtgctgtacaccgtgccggccacctgtgtgatcgcctgctacttttacgaacgcctcaacatggattactggaagatcctggcggcgcagcacaagtgcaaaatgaacaaccagactaaaacgctggactgcctgatggccgcctccatccccgccgtggagatcttcatggtgaagatctttatgctgctggtggtggggatcaccagcgggatgtggatttggacctccaagactctgcagtcctggcagcaggtgtgcagccgtaggttaaagaagaagagccggagaaaaccggccagcgtgatcaccagcggtgggatttacaaaaaagcccagcatccccagaaaactcaccacgggaaatatgagatccctgcccagtcgcccacctgcgtgtgaacagggctggagggaagggcacaggggcgcccggagctaagatgtggtgcttttcttggttgtgtttttctttcttcttcttctttttttttttttataaaagcaaaagagaaatacataaaaaagtgtttaccctgaaattcaggatgctgtgatacactgaaaggaaaaatgtacttaaagggttttgttttgttttggttttccagcgaagggaagctcctccagtgaagtagcctcttgtgtaactaatttgtggtaaagtagttgattcagccctcagaagaaaacttttgtttagagccctccctaaatatacatctgtgtatttgagttggctttgctacccatttacaaataagaggacagataactgctttgcaaattcaagagcctcccctgggttaacaaatgagccatccccagggcccacccccaggaaggccacagtgctgggcggcatccctgcagaggaaagacaggacccggggcccgcctcacaccccagtggatttggagttgcttaaaatagactccggccttcaccaatagtctctctgcaagacagaaacctccatcaaacctcacatttgtgaactcaaacgatgtgcaatacatttttttctctttccttgaaaataaaaagagaaacaagtattttgctatatataaagacaacaaaagaaatctcctaacaaaagaactaagaggcccagccctcagaaacccttcagtgctacattttgtggctttttaatggaaaccaagccaatgttatagacgtttggactgatttgtggaaaggaggggggaagagggagaaggatcattcaaaagttacccaaagggcttattgactctttctattgttaaacaaatgatttccacaaacagatcaggaagcactaggttggcagagacactttgtctagtgtattctcttcacagtgccaggaaagagtggtttctgcgtgtgtatatttgtaatatatgatatttttcatgctccactattttattaaaaataaaatatgttctttagtttgctgctaaaaaaa

SEQ ID NO: 186 - DNA, 1746 bp, Artificial Sequence
  Description of Artificial Sequence:  note = Synthetic Construct
atgcagcgcccgggcccccgcctgtggctggtcctgcaggtgatgggctcgtgcgccgccatcagctccatggacatggagcgcccgggcgacggcaaatgccagcccatcgagatcccgatgtgcaaggacatcggctacaacatgactcgtatgcccaacctgatgggccacgagaaccagcgcgaggcagccatccagttgcacgagttcgcgccgctggtggagtacggctgccacggccacctccgcttcttcctgtgctcgctgtacgcgccgatgtgcaccgagcaggtctctacccccatccccgcctgccgggtcatgtgcgagcaggcccggctcaagtgctccccgattatggagcagttcaacttcaagtggcccgactccctggactgccggaaactccccaacaagaacgaccccaactacctgtgcatggaggcgcccaacaacggctcggacgagcccacccggggctcgggcctgttcccgccgctgttccggccgcagcggccccacagcgcgcaggagcacccgctgaaggacgggggccccgggcgcggcggctgcgacaacccgggcaagttccaccacgtggagaagagcgcgtcgtgcgcgccgctctgcacgcccggcgtggacgtgtactggagccgcgaggacaagcgcttcgcagtggtctggctggccatctgggcggtgctgtgcttcttctccagcgccttcaccgtgctcaccttcctcatcgacccggcccgcttccgctaccccgagcgccccatcatcttcctctccatgtgctactgcgtctactccgtgggctacctcatccgcctcttcgccggcgccgagagcatcgcctgcgaccgggacagcggccagctctatgtcatccaggagggactggagagcaccggctgcacgctggtcttcctggtcctctactacttcggcatggccagctcgctgtggtgggtggtcctcacgctcacctggttcctggccgccggcaagaagtggggccacgaggccatcgaagccaacagcagctacttccacctggcagcctgggccatcccggcggtgaagaccatcctgatcctggtcatgcgcagggtggcgggggacgagctcaccggggtctgctacgtgggcagcatggacgtcaacgcgctcaccggcttcgtgctcattcccctggcctgctacctggtcatcggcacgtccttcatcctctcgggcttcgtggccctgttccacatccggagggtgatgaagacgggcggcgagaacacggacaagctggagaagctcatggtgcgtatcgggctcttctctgtgctgtacaccgtgccggccacctgtgtgatcgcctgctacttttacgaacgcctcaacatggattactggaagatcctggcggcgcagcacaagtgcaaaatgaacaaccagactaaaacgctggactgcctgatggccgcctccatccccgccgtggagatcttcatggtgaagatctttatgctgctggtggtggggatcaccagcgggatgtggatttggacctccaagactctgcagtcctggcagcaggtgtgcagccgtaggttaaagaagaagagccggagaaaaccggccagcgtgatcaccagcggtgggatttacaaaaaagcccagcatccccagaaaactcaccacgggaaatatgagatccctgcccagtcgcccacctgcgtgtga

SEQ ID NO: 187 - protein, 581 aa, Artificial Sequence
  Description of Artificial Sequence:  note = Synthetic Construct
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFAVVWLAIWAVLCFFSSAFTVLTFLIDPARFRYPERPIIFLSMCYCVYSVGYLIRLFAGAESIACDRDSGQLYVIQEGLESTGCTLVFLVLYYFGMASSLWWVVLTLTWFLAAGKKWGHEAIEANSSYFHLAAWAIPAVKTILILVMRRVAGDELTGVCYVGSMDVNALTGFVLIPLACYLVIGTSFILSGFVALFHIRRVMKTGGENTDKLEKLMVRIGLFSVLYTVPATCVIACYFYERLNMDYWKILAAQHKCKMNNQTKTLDCLMAASIPAVEIFMVKIFMLLVVGITSGMWIWTSKTLQSWQQVCSRRLKKKSRRKPASVITSGGIYKKAQHPQKTHHGKYEIPAQSPTCV

SEQ ID NO: 188 - DNA, 20 bp, Artificial Sequence
  Description of Artificial Sequence:  note = Synthetic Construct
atcttgggctcttgacctga

SEQ ID NO: 189 - DNA, 20 bp, Artificial Sequence
  Description of Artificial Sequence:  note = Synthetic Construct
tcttctccatgggcgttatc

SEQ ID NO: 190 - DNA, 34 bp, Artificial Sequence
  Description of Artificial Sequence:  note = Synthetic Construct
ctgcaagtaaagcaaatcagtgagttgggcagct

SEQ ID NO: 191 - DNA, 20 bp, Artificial Sequence
  Description of Artificial Sequence:  note = Synthetic Construct
ttggccctctgatgaaatgt

SEQ ID NO: 192 - DNA, 20 bp, Artificial Sequence
  Description of Artificial Sequence:  note = Synthetic Construct
acacacccactcctggaaag

SEQ ID NO: 193 - DNA, 20 bp, Artificial Sequence
  Description of Artificial Sequence:  note = Synthetic Construct
ctcattttgattcctctatc

SEQ ID NO: 194 - DNA, 20 bp, Artificial Sequence
  Description of Artificial Sequence:  note = Synthetic Construct
aaattgcatggcctctcaac

SEQ ID NO: 195 - DNA, 20 bp, Artificial Sequence
  Description of Artificial Sequence:  note = Synthetic Construct
tacggaaggcagctttgtct

SEQ ID NO: 196 - DNA, 40 bp, Artificial Sequence
  Description of Artificial Sequence:  note = Synthetic Construct
agaattaatatttgaagatatatattagtttatccaatat

SEQ ID NO: 197 - DNA, 20 bp, Artificial Sequence
  Description of Artificial Sequence:  note = Synthetic Construct
ccatgatagctctcccaagc

SEQ ID NO: 198 - DNA, 20 bp, Artificial Sequence
  Description of Artificial Sequence:  note = Synthetic Construct
tgcacctgaccttctgctaa

SEQ ID NO: 199 - DNA, 46 bp, Artificial Sequence
  Description of Artificial Sequence:  note = Synthetic Construct
acagtgtcctggacatccacaggctctcagacaaagataacattat

SEQ ID NO: 200 - DNA, 21 bp, Artificial Sequence
  Description of Artificial Sequence:  note = Synthetic Construct
tggagaggtggacagataagg

SEQ ID NO: 201 - DNA, 20 bp, Artificial Sequence
  Description of Artificial Sequence:  note = Synthetic Construct
ataagcgctcaaaggctgga

SEQ ID NO: 202 - DNA, 46 bp, Artificial Sequence
  Description of Artificial Sequence:  note = Synthetic Construct
tgcagcgttttcggcgcttcctgcaggcgagacagatttgcacgcc

SEQ ID NO: 203 - DNA, 20 bp, Artificial Sequence
  Description of Artificial Sequence:  note = Synthetic Construct
ccttggatgggtattccaga

SEQ ID NO: 204 - DNA, 20 bp, Artificial Sequence
  Description of Artificial Sequence:  note = Synthetic Construct
cctgaggcacagtctgatga

SEQ ID NO: 205 - DNA, 40 bp, Artificial Sequence
  Description of Artificial Sequence:  note = Synthetic Construct
agtgtcttttgaattattttagtgaaacgatgcaggttta